(19) United States
(12) Patent Application Publication
(10) Pub. No.: US 2026/0366062 A1
(43) Pub. Date: Nov. 19, 2026

(54) MULTIPLEXED DETECTION OF NUCLEIC ACID TARGETS

(71) Applicant: Advanced Cell Diagnostics, Inc., Newark, CA (US)

(72) Inventors: Ching-Wei Chang, Newark, CA (US); Steve Zhou, Newark, CA (US); Ji Zhang, Newark, CA (US); Alvason Li, Newark, CA (US); Li-Chong Wang, Newark, CA (US); Maithreyan Srinivasan, Newark, CA (US)

(21) Appl. No.: 19/136,401

(22) PCT Filed: Dec. 7, 2023
(86) PCT No.: PCT/US2023/082958

§ 371 Date: Jun. 6, 2025

Related U.S. Application Data
(60) Provisional application No. 63/386,425, filed on Dec. 7, 2022.

(51) Int. Cl. C12Q 1/682; C12Q 1/6841

(52) U.S. Cl. CPC C12Q 1/682; C12Q 1/6841

(57) ABSTRACT

The present disclosure provides methods, compositions, and kits for the multiplexed detection and corresponding expression analysis of nucleic acids in biological samples. The methods comprise hybridizing to the targets a plurality of target probe sets and a plurality of signal generating complexes (SGCs), each comprising a plurality of detectable labels, and determining a first identifier based on the number of distinct detectable labels in each SGC. The signals are then removed from the sample and the hybridization cycle is repeated with a second plurality of probes or SGCs to determine a second identifier.

### CROSS REFERENCE TO RELATED APPLICATIONS

This application claims priority to and the benefit of U.S. Provisional Patent Application No. 63/386,425 filed Dec. 7, 2022, which is incorporated herein by reference in its entirety and for all purposes.

### FIELD

The present disclosure provides methods, compositions, and kits for the multiplexed detection and corresponding expression analysis of nucleic acids in biological samples.

### BACKGROUND

RNA in situ hybridization (ISH) is a molecular biology technique widely used to measure and localize specific RNA sequences, for example, messenger RNAs (mRNAs), long non-coding RNAs (lncRNAs), and microRNAs (miRNAs) within cells, such as circulating tumor cells (CTCs) or tissue sections, while preserving the cellular and tissue context. RNA ISH therefore provides for spatial-temporal visualization as well as quantification of gene expression within cells and tissues. It has wide applications in research and in diagnostics (Hu et al., Biomark. Res. 2(1):1-13, doi: 10.1186/2050-7771-2-3 (2014); Ratan et al., Cureus 9(6):e1325. doi: 10.7759/cureus.1325 (2017); Weier et al., Expert Rev. Mol. Diagn. 2(2):109-119 (2002))). Fluorescent RNA ISH utilizes fluorescent dyes and fluorescent microscopes for RNA labeling and detection, respectively. Fluorescent RNA ISH typically provides for limited multiplexing of four to five target sequences. The limited multiplexing capability is largely due to the small number of spectrally distinct fluorescent dyes that can be distinguished by the optical systems of the fluorescence microscope. Higher level of multiplexing is highly desirable in areas such as generating cell and tissue maps to understand complex biological systems, particularly in human health and disease.

### SUMMARY

In one aspect, disclosed herein is a method for detecting a plurality of target nucleic acids in a sample, the method comprising:
contacting a sample comprising a plurality of target nucleic acids with a plurality of target probe sets, wherein each target probe set is complementary to a target nucleic acid;
contacting the sample with a first plurality of signal generating complexes (SGCs) capable of hybridizing to at least one target probe set, wherein each SGC comprises a plurality of spatially distributed label probes comprising detectable labels;
obtaining a first independent identifier corresponding to at least one target nucleic acid, wherein the first independent identifier is based on the number of distinct detectable labels present in each SGC of the first plurality of SGCs;
removing signals generated by the detectable labels from the sample;
contacting the sample with at least a second plurality of SGCs capable of hybridizing to at least one target probe set, wherein each SGC comprises a plurality of spatially distributed label probes comprising detectable labels; and
obtaining a second independent identifier corresponding to at least one target nucleic acid, wherein the second independent identifier is based on the number of distinct detectable labels present in each SGC of the second plurality of SGCs.

In some embodiments, the method further comprises identifying a target nucleic acid of the plurality of target nucleic acids based on a combination of the first and second independent identifiers.

In some embodiments, the target nucleic acid is identified independent of the order by which the first and second independent identifiers are obtained.

In some embodiments, the first and second independent identifiers are based on the same number of distinct detectable labels present in the first and second plurality of SGCs, respectively. In some embodiments, the first and second independent identifiers are based on a different number of distinct detectable labels present in the first and second plurality of SGCs, respectively. In some embodiments, the independent identifier is based on the number of distinct detectable labels present in a combination of at least two SGCs.

In some embodiments, the spatially distributed label probes comprise two distinct detectable labels. In some embodiments, the spatially distributed label probes comprise three distinct detectable labels. In some embodiments, the spatially distributed label probes comprise four distinct detectable labels. In some embodiments, the spatially distributed label probes comprise five distinct detectable labels. In some embodiments, the spatially distributed label probes comprise six distinct detectable labels.

In some embodiments, obtaining an independent identifier comprises obtaining an image of the sample and detecting signals generated by the detectable labels.

In some embodiments, the method further comprises:
removing the signals generated by the detectable labels of the second plurality of SGCs from the sample;
contacting the sample with at least a third plurality of SGCs capable of hybridizing to at least one target probe set, wherein each SGC comprises a plurality of spatially distributed label probes comprising detectable labels; and
obtaining a third independent identifier corresponding to at least one target nucleic acid, wherein the third independent identifier is based on the number of distinct detectable labels present in each SGC of the third plurality of SGCs.

In some embodiments, each target probe set comprises two or more target probes capable of hybridizing to a target nucleic acid. In some embodiments, each target probe in the target probe set comprises a T section complementary to a region of a target nucleic acid and an L section complementary to a region of an SGC. In some embodiments, each T section is complementary to a non-overlapping region of a target nucleic acid and each L section is complementary to a non-overlapping region of an SGC. In some embodiments, the T section of at least one of the target probes in the target probe set is 3′ of its L section. In some embodiments, the T section of at least one of the target probes in the target probe set is 5′ of its L section.

In some embodiments, each SGC comprises one or more of an amplifier, a pre-amplifier, and a pre-pre-amplifier. In some embodiments, each label probe in the plurality of label probes comprises a binding site for the amplifier. In some embodiments, the amplifier comprises at least one binding site for the pre-amplifier and a plurality of binding sites for the plurality of label probes. In some embodiments, the pre-amplifier comprises at least one binding site for the L section of the target probe and at least one binding site for the amplifier. In some embodiments, the pre-amplifier comprises at least one binding site for the pre-pre-amplifier and at least one binding side for the amplifier. In some embodiments, the at least one binding site for the L section of the target probe in the pre-amplifier or the pre-pre-amplifier is distinct for each SGC corresponding to a target nucleic acid. In some embodiments, the at least one binding site for the L section of the target probe in the pre-amplifier or the pre-pre-amplifier is the same for each SGC corresponding to a target nucleic acid.

In some embodiments, removing the signals generated by the detectable labels comprises removing the SGCs. In some embodiments, removing the SGCs comprises treatment with an acid reagent that disrupts hybridization between the SGCs and the target nucleic acids. In some embodiments, the acid reagent comprises formic acid, acetic acid, propionic acid, butyric acid, valeric acid, oxalic acid, malonic acid, succinic acid, malic acid, tartaric acid, citric acid. In some embodiments, after the first plurality of SGCs are removed, the second plurality of SGCs are hybridized to the same target probe set. In some embodiments, after the first plurality of SGCs are removed, the second plurality of SGCs are hybridized to a different target probe set.

In some embodiments, removing the signals generated by the detectable labels comprises removing the detectable labels from the plurality of label probes using a cleavage reagent. In some embodiments, after the detectable labels are removed, the second plurality of SGCs are hybridized to a different target probe set.

In some embodiments, after the step of removing signals generated by the detectable labels from the sample, the method further comprises a step of contacting the sample with a second plurality of target probe sets, wherein each target probe set in the second plurality of target probe sets is complementary to a target nucleic acid.

In some embodiments, the sample comprises a cell. In some embodiments, the method further comprises fixing and/or permeabilizing the cell.

In some embodiments, the target nucleic acid is RNA. In some embodiments, the method further comprises detecting at least one non-nucleic acid target in the sample. In some embodiments, the non-nucleic acid target is a protein.

In one aspect, disclosed herein is a method for detecting a plurality of target nucleic acids in a sample, the method comprising:
contacting a sample comprising a plurality of target nucleic acids with a plurality of target probe sets, wherein each target probe set is complementary to a target nucleic acid;
contacting the sample with a plurality of amplification complexes capable of hybridizing to at least one target probe set, wherein each amplification complex comprises a plurality of spatially distributed binding sites for label probes;
contacting the sample with a first plurality of label probes comprising detectable labels, which hybridize to binding sites on the amplification complexes to generate a first plurality of signal generating complexes (SGCs);
obtaining a first independent identifier corresponding to at least one target nucleic acid, wherein the first independent identifier is based on the number of distinct detectable labels present in each SGC of the first plurality of SGCs;
removing signals generated by the detectable labels from the sample;
contacting the sample with at least a second plurality of label probes comprising detectable labels, which hybridize to binding sites on the amplification complexes to generate a second plurality of SGCs; and
obtaining a second independent identifier corresponding to at least one target nucleic acid, wherein the second independent identifier is based on the number of distinct detectable labels present in each SGC of the second plurality of SGCs.

In some embodiments, the method further comprises identifying a target nucleic acid of the plurality of target nucleic acids based on a combination of the first and second independent identifiers. In some embodiments, the target nucleic acid is identified independent of the order by which the first and second independent identifiers are obtained.

In some embodiments, the first and second independent identifiers are based on the same number of distinct detectable labels present in the first and second plurality of SGCs, In some embodiments, the first and second independent identifiers are based on a different number of distinct detectable labels present in the first and second plurality of SGCs, respectively. In some embodiments, the independent identifier is based on the number of distinct detectable labels present in a combination of at least two SGCs.

In some embodiments, the spatially distributed label probes comprise two distinct detectable labels. In some embodiments, the spatially distributed label probes comprise three distinct detectable labels. In some embodiments, the spatially distributed label probes comprise four distinct detectable labels. In some embodiments, the spatially distributed label probes comprise five distinct detectable labels. In some embodiments, the spatially distributed label probes comprise six distinct detectable labels.

In some embodiments, obtaining an independent identifier comprises obtaining an image of the sample and detecting signals generated by the detectable labels.

In some embodiments, the method further comprises:
removing the signals generated by the detectable labels of the second plurality of SGCs from the sample;
contacting the sample with at least a third plurality of label probes comprising detectable labels, which hybridize to binding sites on the amplification complexes to generate a third plurality of SGCs; and
obtaining a third independent identifier corresponding to at least one target nucleic acid, wherein the third independent identifier is based on the number of distinct detectable labels present in each SGC of the third plurality of SGCs.

In some embodiments, each target probe set comprises two or more target probes capable of hybridizing to a target nucleic acid. In some embodiments, each target probe in the target probe set comprises a T section complementary to a region of a target nucleic acid and an L section complementary to a region of an amplification complex. In some embodiments, each T section is complementary to a non-overlapping region of a target nucleic acid and each L section is complementary to a non-overlapping region of an amplification complex. In some embodiments, the T section of at least one of the target probes in the target probe set is 3′ of its L section. In some embodiments, the T section of at least one of the target probes in the target probe set is 5′ of its L section.

In some embodiments, each amplification complex comprises one or more of an amplifier, a pre-amplifier, and a pre-pre-amplifier. In some embodiments, each label probe in the plurality of label probes comprises a binding site for the amplifier. In some embodiments, the amplifier comprises at least one binding site for the pre-amplifier and a plurality of binding sites for the plurality of label probes. In some embodiments, the pre-amplifier comprises at least one binding site for the L section of the target probe and at least one binding site for the amplifier. In some embodiments, the pre-amplifier comprises at least one binding site for the pre-pre-amplifier and at least one binding side for the amplifier. In some embodiments, the at least one binding site for the L section of the target probe in the pre-amplifier or the pre-pre-amplifier is distinct for each amplification complex corresponding to a target nucleic acid. In some embodiments, the at least one binding site for the L section of the target probe in the pre-amplifier or the pre-pre-amplifier is the same for each amplification complex corresponding to a target nucleic acid.

In some embodiments, removing the signals generated by the detectable labels comprises removing the detectable labels from the plurality of label probes using a cleavage reagent.

In some embodiments, after the detectable labels from the first plurality of SGCs are removed, the second plurality of label probes are hybridized to different amplification complexes to generate the second plurality of SGCs. In some embodiments, after the step of removing signals generated by the detectable labels from the first plurality of label probes from the sample, the method further comprises a step of contacting the sample with a second plurality of target probe sets, wherein each target probe set in the second plurality of target probe sets is complementary to a target nucleic acid.

In some embodiments, the sample comprises a cell. In some embodiments, the method further comprises fixing and/or permeabilizing the cell.

In some embodiments, the target nucleic acid is RNA. In some embodiments, the method further comprises detecting at least one non-nucleic acid target in the sample. In some embodiments, the non-nucleic acid target is a protein.

In one aspect, disclosed herein is a kit for carrying out any of the methods disclosed herein.

### BRIEF DESCRIPTION OF THE DRAWINGS

FIGS. 1A-1B show exemplary configurations of probes for detecting a target nucleic acid. In the configuration shown in FIG. 1A, each individual target probe has a target (T) segment complementary to the target nucleic acid (i.e., a segment of the target probe that can hybridize to the target nucleic acid) and a label (L) segment complementary to a component of a Signal Generating Complex (SGC) (i.e., a segment of the target probe that can hybridize to a component of the SGC). Each SGC comprises multiple layers of components, such as amplifiers (AP) and pre-amplifiers (PA) that assemble into a tree-like structure which is capable of carrying many label probes (LP) on its “branches.” As shown in FIG. 1B, if the target sequence is sufficiently long, many TP sets and associated SGCs can be assembled on the target nucleic acid to generate a detectable signal that appears in an imaging system as a discrete “dot.”

FIGS. 2A-2B show two exemplary configurations of probes for detecting a target nucleic acid. The configurations utilize SGCs comprising LPs, APs, PAs, TPs, as shown in FIGS. 1A-1B, except that assembly of the SGC utilizes a Collaboration Amplifier (COM). The COMs bind to two pre-amplifiers (PAs) and to an amplifier (AP) for assembly of the SGC. FIGS. 2A-2B show two different configurations of the target probe binding to the target nucleic acid. The configurations allow more LPs to be incorporated into one SGC. Such configurations are suitable, e.g., for detecting short target sequences, because a detectable signal can be generated with a single SGC.

FIG. 3 shows an exemplary configuration for multiplex detection of target nucleic acids. In this embodiment, each SGC carries the same LPs with a specific label, where different SGCs carry distinct labels. Components of each SGC (such as PAs, APs, LPs, etc.) are uniquely associated with the SGC. The SGCs are designed so that the components of a target-specific SGC hybridize to each other to assemble the SGC but cannot cross-hybridize to any components of any other SGC. In FIG. 3, two target nucleic acids are shown bound to the respective SGCs. For one target nucleic acid (upper target in FIG. 3), SGCs for the target comprise four labels (L1, L2, L3, and L4 that are specific to this SGC). For a second target (lower target in FIG. 3), the code for the target nucleic acid, the SGCs for the target nucleic acid comprise two labels (L2 and L4 that are specific to this SGC).

FIGS. 4A-4G illustrate various exemplary embodiments for multiplex detection of target nucleic acids. FIG. 4A illustrates one embodiment of sub-SGC implementation, where the SGC ID code is implemented on the amplifier (AP) molecule. As shown in FIG. 4A, an AP has one region designed to bind to the amplifier anchor (AA) on the PA molecule (i.e., binding site for the amplifier on the pre-amplifier) and another region comprising multiple segments of label probe anchors (LAs) (i.e., binding sites for the label probes). In this embodiment, a mixture of different LAs are designed according to the unique identification code of the SGC. For example, if the identifier of the SGC is based on three labels, then equal number of LAs for each of the three labels are made on the AP molecule, which will bind a designed number of desired LPs to generate the identifier in the assay. In FIG. 4A, the code for the SGC shown uses labels L1, L3, and L4 (alphanumeric code C, see Table 1a).

FIG. 4B illustrates another embodiment of sub-SGC implementation, where the SGC ID code is implemented on the pre-amplifier (PA) molecule. As shown, N “pure” AP molecules are made, each carrying the same type of LP. A PA molecule has one region designed to bind to the SA (i.e., binding site for the PA on the TP; see FIG. 1A) of a TP set and another region comprising multiple segments of AAs (i.e., binding sites for amplifiers on the pre-amplifier). In this embodiment, a mixture of different AAs is designed according to the unique identification code of the SGC. For example, if the ID code of the SGC includes the two labels L2 and L4, then equal number of AAs for APs carrying LP2 and LP4 are made on the PA molecule, which will bind a designed number of desired LPs to generate the identifier in the assay. In FIG. 4B, the identifier of the SGC is shown using L1, L3, and L4 (alphanumeric code C, see Table 1a).

FIG. 4C illustrates another embodiment of sub-SGC implementation, where the SGC ID code is implemented on the LP molecule. In this embodiment, LP molecules binding to the same SGC can be mixtures of LPs each conjugated to a different label according to a predefined code book. For example, as shown in FIG. 4C, SGC5 LPs are a mixture of LPs conjugated to three different labels (L1, L3, and L4). An advantage of this embodiment is that the “coloring” of the SGC complex by the LPs will be completely randomized, which can further help to reduce coding errors. A partial LP mixing code book is shown on the right of FIG. 4C, with 7 different exemplary SGC identifiers shown using 4 labels.

FIG. 4D shows an embodiment of FIG. 4C in more detail. For each SGC, a specific label anchor (LA, the binding site on the amplifier for the label probe) is assigned so that each SGC for a particular target nucleic acid has a plurality of the same LAs on the amplifier. The level at which combinatorial labeling can be provided is with the label probes (LPs). In this case, SGC5 is illustrated showing that the amplifiers comprise a plurality of identical LAs, labeled “E.” As shown in FIG. 4D, SGC5 is coded with three distinct labels conjugated to label probes (L1, L3, and L4), all of which have the same binding site for the plurality of “E” LAs on the corresponding amplifier. Therefore, all label probes specific to SGC5 are bound to the amplifiers of SGC5.

FIG. 4E shows an embodiment of FIG. 4C in more detail. The SGC5 of FIG. 4D is shown bound to its respective target nucleic acid, with the label probes having an “E” binding site bound to the respective “E” LAs of the SGC5 amplifiers. Also shown are two additional exemplary SGCs bound to their respective target nucleic acids. SGC1, coded as shown in FIG. 4D, comprises a plurality of identical LAs, labeled “A.” SGC1 is coded with one label (L1), which has the binding site for the plurality of “A” LAs of the SGC1 amplifiers. Therefore, L1 is bound to the amplifiers of SGC1, thereby identifying the SGC1 target nucleic acid with the independent identifier based on L1 (alphanumeric code 1, see Table 1a). SGC3, coded as shown in FIG. 4D, comprises a plurality of identical LAs, labeled “C.” SGC3 is coded with two distinct label probes (L1 and L2), both of which have the same binding site for the plurality of “C” LAs on the corresponding SGC3 amplifiers. Therefore, both label probes are bound to the amplifiers of SGC3, thereby labeling the SGC3 target nucleic acid with the independent identifier based on L1 and L2 (alphanumeric code A, see Table 1a).

FIG. 4F shows an embodiment of FIG. 4C in more detail. Once an SGC for a particular target nucleic acid has been designed, the identifier for the target nucleic acid can be readily modified simply by changing the labels on the label probes that bind to the amplifiers of a particular SGC. For example, in FIG. 4D, SGC2 comprises amplifiers with “B” LAs and is coded using label probe L2. The same SGC assembly can be used with respect to the target probes, pre-amplifier, and amplifiers with “B” LAs, but instead of using “B” LA-binding label probes with only L2 as in FIG. 4D, “B” LA-binding label probes can be used that have a mixture of labels L2 and L3, such that SGC2 is now coded with both labels (alphanumeric code 8, see Table 1a). Thus, L2 and L3 are bound to “B” LAs on the SGC2 amplifiers. Similarly, SGC5 comprising amplifiers with “E” LAs is now coded in FIG. 4F by using label probes with “E” LA-binding label probes that have a mixture of labels L2, L3, and L4 (alphanumeric code E, see Table 1a), instead of labels L1, L3, and L4 (alphanumeric code C, see Table 1a) as shown in FIG. 4D.

FIG. 4G shows an embodiment of FIG. 4C in more detail. Two target nucleic acids are shown with two bound SGCs, SGC2 and SGC5. SGC2 is coded with an identifier based on labels L2 and L3 (alphanumeric code 8, see Table 1a), and SGC5 is coded with an identifier based on labels L2, L3, and L4 (alphanumeric code E, see Table 1a). In this case, where all of the label probes bind to the respective LAs, “B” LAs in the case of SGC2 and “E” LAs in the case of SGC5, and assuming that the SGC2 and SGC5 have approximately the same number of LAs in the respective SGCs, the number of respective labels that can bind to SGC2 will be higher than the number of respective labels that bind to SGC5 (i.e., the 2 distinct labels for SGC2 and the 3 distinct labels for SGC5 will be bound to the same number of sites, resulting in a higher number of L2 and L3 being bound to SGC2 than SGC5 since some of the SGC5 sites are occupied by L4). If desired, the number of labels (and therefore intensity of signal) can be normalized by including “blank” label probes, i.e., probes having a binding site for the respective LAs (in this case “B” for SGC2 and “E” for SGC5) but without a label. For example, if it is desired to compare SGC2 and SGC5 with equal intensity signals for the respective labels, ⅓ “blank” label probes can be included with the mixture of “B” LA-specific probes so that the intensity of L2 and L3 will be the same on both SGCs (i.e., ⅓ of SGC2 occupied by “blank” label probes and ⅓ of SGC5 occupied by L4). In another example, if a multiplex assay is being performed where some SGCs include 4 labels, then the assay can be performed so that the same proportion of “blank” label probes are included in the label probe sets using less than 4 labels, for example, ½ “blank” label probes can be included with the SG2-specific label probes and ¼ “blank” label probes to be included with the SGC5-specific label probes so that the amount of each distinct label probe bound to the respective SGCs is the same on each SGC.

FIG. 5 shows two configurations of assembly of SGCs on a target nucleic acid. In the lower panel of FIG. 5, the SGCs providing the same label are shown binding in a group next to each other. In the upper panel of FIG. 5, the SGCs providing different labels are shown with binding sites on the target nucleic acid intermingled or intertwined. The intermingling of target probe binding sites on the target nucleic acid for different labels are advantageous because, if different SGC types are positioned apart, in separate groups, a certain section of the target may be blocked or masked, thereby preventing attachment of one specific SGC type, which will result in miscoding.

FIGS. 6A-6B demonstrate configurations for reducing miscoding for multiplex detection of target nucleic acids. As shown in FIG. 6A, the particular SGC is miscoded because the PA is truncated, which could occur during manufacturing of the PA. In this case in FIG. 6A, L4 is not bound due to truncation. As shown in FIG. 6B, the same truncation will not cause miscoding if the labels are intertwined or intermingled on the PA. Arranging different labels into alternating positions reduces the chance of miscoding.

FIGS. 7A-7B show a method to minimize potential miscoding caused by truncation by randomizing the position of different labels on the AP or PA. As illustrated in FIGS. 7A-7B, the multiplexing channel ID is encoded on the AP molecule. In FIG. 7A, different label probes are positioned on each AP in exactly the same way, that is, each amplifier in the SGC is the same. Truncation of some of the APs can cause substantial reduction in certain labels being bound to the target nucleic acid compared to other labels on different positions of the AP. This imbalance increases the chance of miscoding. In the most severe case, truncation could cause the loss of all copies of one certain label, leading to an outright miscode. In FIG. 7B, locations of different labels on the APs are randomized. The APs are provided as a plurality of amplifiers, where a mix of non-identical amplifiers is included, where the position of LAs for specific label probes are distributed differently and can be randomized on the non-identical amplifiers. Truncation therefore does not cause a large bias in the number of labels in the SGC.

FIG. 8 shows an exemplary embodiment of the methods disclosed herein for multiplexed detection of target nucleic acids, by generation of two separate independent identifiers of each target nucleic acid using two separate rounds of labeling and detection. The identifiers are incorporated at the label-probe level, using the same regions of each target analyte in each round. In the first round, each analyte (i.e. “gene” in this figure) is labeled with one type of the unique SGCs (i.e. “trees” in this figure), each of which provides signals that forms a unique combinatorial code. For example, for SGC “E” with alphanumeric code E, the SGC includes three different label probes, but not the fourth. Once these signals are detected and recorded to generate the first independent identifier of each target gene, the signals are removed, and the analyte is labeled with another type of SGC. The second set of signals is then detected and recorded. This procedure can continue with more rounds. The expanded code can then be used to identify the analyte. Note in this figure the individual labels are not distinguished for simplicity. Round 2 can target either the same sequence of the target as in the first round, or a different sequence. If the same sequence is targeted, the T-sections of the target probes can be the same as those used in the previous round, and the entire signal-generating complex is removed between each round. If a different sequence is targeted, the T-sections of the target probes will be different, and only the fluorophore need be cleaved from the SGCs between each round.

FIG. 9 shows a representative illustration of an embodiment using different combinations of labels at the SGC level. In the first round, each analyte (i.e. “gene” in this figure) is labeled with one combination of the unique SGCs (i.e. “trees” in this figure), each of which provides one signal only. For example, SGC “α” contains the first signal only but not others. Once these signals (i.e., the first independent identifier) are detected and recorded, either the signals or the SGCs are removed, and analyte is labeled with another combination of unique SGCs for the second round. The second set of the combinatorial code is then detected and recorded. This procedure can continue with more rounds. The expanded combinatorial code can then be used to identify the analyte. Round 2 can target either the same sequence of the target as in the first round, or a different sequence. If the same sequence is targeted, the T-sections of the target probes can be either the same as those used in the previous round or different from those used in the previous round, and the entire signal-generating complex is removed between each round. If a different sequence is targeted, the T-sections of the target probes will be different, and only the fluorophore needs be cleaved from the SGCs between each round.

FIGS. 10A-10C show an exemplary assay workflows for the methods disclosed herein.

FIG. 11 shows a representative image demonstrating detection of a single gene (RNA transcript), Ube, in a two-cycle experiment according to the methods disclosed herein. In each round of labeling, the independent identifier in this experiment was based on a single fluorescent label. The signals detected in the first round are indicated by triangles in the image, and signals detected in the second round are indicated by squares. Locations with overlapping triangles and squares are the locations where the target gene, was detected.

FIGS. 12A-12C show representative images from a single two-cycle experiment to detect three genes (RNA transcripts Gadph, Sdha, and Ubc). Each target was assigned the same first independent identifier based on a single fluorescent label, and each target was assigned a different second independent identifier based on a single fluorescent label. FIG. 12A shows detection of Gadph, FIG. 12B shows detection of Sdha, and FIG. 12C shows detection of Ubc. In each image, the signals detected in the first cycle represent all three genes, and are shown in triangles. In FIG. 12A, signals detected in the second cycle are indicated by circles, and locations with overlapping triangles and circles are the locations where the combined first and second identifiers show that Gadph was detected. In FIG. 12B, signals detected in the second cycle are indicated by crosses, and locations with overlapping triangles and crosses are the locations where the combined first and second identifiers show that Sdha was detected. In FIG. 12C, signals detected in the second cycle are indicated by squares, and locations with overlapping triangles and squares are the locations where the combined first and second identifiers show that Ubc was detected.

FIGS. 13A-13C show representative images from a first two-cycle experiment to detect one gene (RNA transcript Hs-PPIB), with the first and second identifiers each based on two fluorescent labels. Further details can be found in Example 3. FIG. 13A shows images from the first round of labeling and detection, with panels showing detection of DAPI (upper left), Dylight 550 (upper middle, no label detected), Dylight 650 (upper right, label detected), Alexa Fluoro 488 (lower left, no label detected), Alexa Fluoro 750 (lower middle, label detected), and an overlay (lower right). FIG. 13B shows images from the second round of labeling and detection, with panels showing detection of DAPI (upper left), Dylight 550 (upper middle, label detected), Dylight 650 (upper right, label detected), Alexa Fluoro 750 (lower left, no label detected), Alexa Fluoro 488 (lower middle, no label detected), and an overlay (lower right). FIG. 13C is an overlay of all signals detected in the first and second rounds, with shaded circles showing “dots” having the complete identifier “code” for the target.

FIGS. 14A-14C show representative images from a second two-cycle experiment to detect one target nucleic acid (RNA transcript Hs-PPIB), with the first and second identifiers each based on two fluorescent labels, where the order of the identifiers was reversed compared to those in the experiments shown in FIGS. 13A-13C. Further details can be found in Example 3. FIG. 14A shows images from the first round of labeling and detection, with panels showing detection of DAPI (upper left), Dylight 550 (upper middle, label detected), Dylight 650 (upper right, label detected), Alexa Fluoro 488 (lower left, no label detected), Alexa Fluoro 750 (lower middle, no label detected), and an overlay (lower right). FIG. 14B shows images from the second round of labeling and detection, with panels showing detection of DAPI (upper left), Dylight 550 (upper middle, no label detected), Dylight 650 (upper right, label detected), Alexa Fluoro 750 (lower left, label detected), Alexa Fluoro 488 (lower middle, no label detected), and an overlay (lower right). FIG. 14C is an overlay of all signals detected in the first and second rounds, with shaded circles showing “dots” having the complete identifier “code” for the target.

FIGS. 15A-15C show representative images from a single ISH labeling experiment to detect one target nucleic acid (RNA transcript Hs-PPIB) in Hela cells, to demonstrate that individual PPIB signals (coded with alphanumeric Code 8) can be detected in any order to generate an independent identifier, as further described in Example 4. FIG. 15A shows images detected using two different Excitation/Emission filter sequences. FIG. 15B shows an overlay of the images from each sequence. FIG. 15C presents overlays showing individual “dots” for detection of PPIB.

FIGS. 16A-16B show exemplary embodiments of the methods disclosed herein for multiplexed detection of target nucleic acids, by generation of two separate independent identifiers of each target nucleic acid using two separate rounds of labeling and detection. In FIG. 16A, the identifiers are incorporated at the label-probe level, using the same regions of each target analyte in each round. In the first round, each analyte (i.e., “gene” in this figure) is labeled with one type of the unique SGCs (i.e., “trees” in this figure), each of which provides signals that forms a unique combinatorial code. For example, for SGC “E” with alphanumeric code E, the SGC includes three different label probes (L4, L3, and L2), but not the fourth (L1). Once these signals are detected and recorded to generate the first independent identifier of each target gene, the signals are removed, and the analyte is labeled with another type of SGC. The second set of signals is then detected and recorded. This procedure can continue with more rounds. The expanded code can then be used to identify the analyte. Round 2 can target either the same sequence of the target as in the first round, or a different sequence. If the same sequence is targeted, the T-sections of the target probes can be the same as those used in the previous round, and the entire signal-generating complex is removed between each round. If a different sequence is targeted, the T-sections of the target probes will be different, and only the fluorophore need be cleaved from the SGCs between each round. FIG. 16B shows a representative illustration of an embodiment using different combinations of labels at the SGC level. In the first round, each analyte (i.e., “gene” in this figure) is labeled with one combination of the unique SGCs (i.e., “trees” in this figure), each of which provides one signal only. For example, SGC “α” contains the first signal only but not others. Once these signals (i.e., the first independent identifier) are detected and recorded, either the signals or the SGCs are removed, and analyte is labeled with another combination of unique SGCs for the second round. The second set of the combinatorial code is then detected and recorded. This procedure can continue with more rounds. The expanded combinatorial code can then be used to identify the analyte. Round 2 can target either the same sequence of the target as in the first round, or a different sequence. If the same sequence is targeted, the T-sections of the target probes can be either the same as those used in the previous round or different from those used in the previous round, and the entire signal-generating complex is removed between each round. If a different sequence is targeted, the T-sections of the target probes will be different, and only the fluorescent signals need to be removed from the SGCs between each round.

FIG. 17 shows representative graphs of spatial transcriptomic profiling in a 1 month old C57BL/6 mouse brain (top, motor cortex) and a 1 year old C57BL/6 mouse brain (bottom, lateral cortex). Each of the 100 target genes is differentially expressed by a unique cell type in the mouse brain and thus can be used to mark the particular cell type. The correlation coefficient (R value) ranges from 0.6~0.81, demonstrating that the disclosed method (referred to as “Ultraplex assay”) can effectively profile the gene expressions of mouse brain cells at single-cell level.

FIG. 18 shows representative graphs of data pertaining to minor gene expression discrepancies between the Ultraplex assay and the control assay tested in FIG. 17. The data indicated that the Ultraplex assay resulted in more accurate measurements of the gene expressions in mouse brain (as suggested by the Hiplex assay performed in the same mouse brain tissues against these disputed genes).

FIG. 19 shows representative graphs correlating gene expression data from the Ultraplex assay with annotated cell types from control assays. Between 10-16 major cell types in the different mouse brain regions were correctly identified using the Ultraplex assay, indicating consistent spatial distribution across the mouse brain datasets.

### DETAILED DESCRIPTION

Disclosed herein are methods for multiplex analysis of nucleic acids by in situ hybridization. The disclosed methods allow the detection of multiple target nucleic acids within the same sample and within the same cell. Each target nucleic acid is labeled with one or more detectable labels that generate a first independent identifier of the target nucleic acid. Removal of the signals followed by a second round of labeling generates a second independent identifier of the target nucleic acid. A combination of the first and second independent identifiers allows for identification of a target nucleic acid, independent of the order by which the first and second independent identifiers are obtained.

### DEFINITIONS

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The terms “detecting” as used herein generally refer to any form of measurement, and include determining whether an element is present or not. This term includes quantitative and/or qualitative determinations.

As used herein, the term “endogenous” refers to the substances originating from within an organism. As used herein, the term “exogenous” refers to the substances originating from outside an organism.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present.

The term “sample” as used herein relates to a material or mixture of materials containing one or more components of interest. The term “sample” includes “biological sample” which refers to a sample obtained from a biological subject, including a sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, cells, and exosomes isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include, but are not limited to, whole blood, partially purified blood, PBMC, tissue biopsies, and the like.

The term “probe” as used herein refers to a capture agent that is directed to a specific target mRNA sequence. Accordingly, each probe of a probe set has a respective target mRNA sequence. In some embodiments, the probe provided herein is a “nucleic acid probe” or “oligonucleotide probe” which refers to a nucleic acid capable of binding to a target nucleic acid of complementary sequence, such as the mRNA biomarkers provided herein, usually through complementary base pairing by forming hydrogen bond. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. The probes can be directly or indirectly labeled with tags, for example, chromophores, lumiphores, or chromogens. By assaying for the presence or absence of the probe, one can detect the presence or absence of a target mRNA biomarker of interest.

As used herein, the term “plurality” means two or more. Thus, a plurality can refer to, for example, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 55 or more, 60 or more, 65 or more, 70 or more, 75 or more, 80 or more, 85 or more, 90 or more, 95 or more, 100 or more, 110 or more, 120 or more, 130 or more, 140 or more, 150 or more, 160 or more, 170 or more, 180 or more, 190 or more, 200 or more, 300 or more, 400 or more, 500 or more, 600 or more, 700 or more, 800 or more, 900 or more, or 1000 or more, or even a greater number, if desired for a particular use.

As used herein, the term “one or more” refers to, for example, 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 55 or more, 60 or more, 65 or more, 70 or more, 75 or more, 80 or more, 85 or more, 90 or more, 95 or more, 100 or more, 110 or more, 120 or more, 130 or more, 140 or more, 150 or more, 160 or more, 170 or more, 180 or more, 190 or more, 200 or more, 300 or more, 400 or more, 500 or more, 600 or more, 700 or more, 800 or more, 900 or more, or 1000 or more, or even a greater number, if desired for a particular use.

As used in the present disclosure and claims, the singular forms “a,” “an,” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### Methods for Multiplexed Nucleic Acid Detection

Disclosed herein are methods for detecting a plurality of target nucleic acids in a sample.

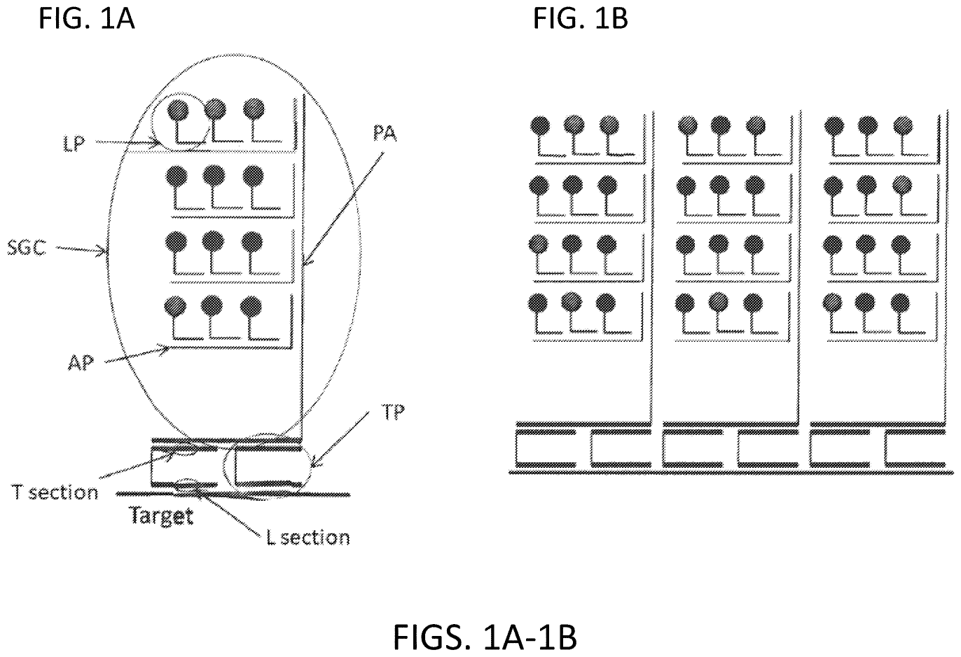

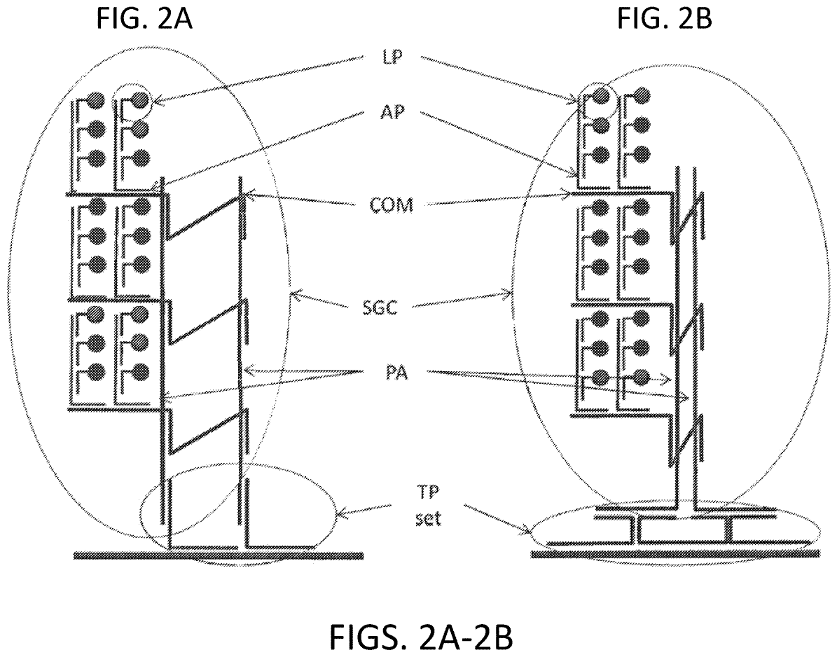

Previously-disclosed methods of detecting target nucleic acids are described in U.S. Pat. No. 7,709,198 and European Patent No. 2500439, which are incorporated herein by reference. These references describe methods of in situ detection of nucleic acid targets in which target probes (TP) are arranged in sets of two or more short probes adjacent to each other when they are hybridized to the target. As shown in FIG. 1A, each individual target probe has a target section (T) complementary to the target and a label section (L) complementary to a component of a Signal Generating Complex (SGC). The T sections of the one or more target probe(s) are complementary to non-overlapping regions of the target nucleic acid, and the L sections of the one or more target probe(s) are complementary to non-overlapping regions of the nucleic acid component of the SGC. Each SGC comprises multiple layers of components, such as Amplifiers (AMP) and Pre-Amplifiers (PA), which assemble into tree-like structures capable of carrying many Label Probes (LP) on their “branches.” As shown in FIG. 1B, if the target sequence is sufficiently long, many TP sets and associated SGCs can be assembled on the target to generate a detectable signal that appears in an imaging system as a discrete “dot.” Manual or computerized dot counting can be conducted to quantify the number of targets in a particular cell in a sample. FIG. 2 shows two additional different configurations of the SGC, in which additional layer(s) of amplification molecules, such as a Collaboration Amplifier (COM), are incorporated to carry more LPs in one SGC. Such configurations are more suitable for detecting short target sequences because a detectable signal can be generated with a single SGC (see, for example, WO 2017/066211, which is incorporated herein by reference).

In many applications, it is valuable to detect multiple targets in the same assay. Previously-disclosed methods address this need in various ways. In a “pooling” approach, each target has unique TP sets but all TP sets have the same L sections, and can therefore be bound to the same SGCs. In this way, a signal is detected when any one of the multiple targets is present. The pooling approach is useful when a group of targets has the same clinical utility or biological functionality. Other methods use a multiplexing approach, in which each target has its own unique SGC that does not cross-hybridize, generating a unique identifiable signal for each target when it is present. The multiplexing approach is useful when each target in the group provides a different clinical or biological indication alone or in combination. In previously-described methods, each unique signal is generated by a large number of LPs carrying the same label. A drawback to this approach is that there are usually a limited number of uniquely identifiable labels. In fluorescent detection modalities, for example, four to six fluorophores at different wavelengths are commonly used. More than six fluorophores in an imaging-based multiplexing system is possible, but can become challenging due to bandwidth limits and cross-talk between wavelengths. This limitation can impose a limit on the number of targets that can be multiplexed in an assay.

The methods disclosed herein allow for multiplexed detection of large numbers of targets in a single sample. Each target in the sample is detected by generating at least two separate independent identifiers of each target in the sample (e.g., two, three, or more independent identifiers), and then combining the separate independent identifiers to obtain a unique identifier for each target in the sample. The combination of multiple independent identifiers allows for simultaneous detection of large numbers of different targets in the same sample. Each independent identifier is based on the number of distinct detectable labels associated with each target. Notably, each target can be identified independent of the order by which the independent identifiers are obtained. As discussed further herein, in some embodiments, the targets are nucleic acids, such as RNA.

In some embodiments, the method comprises first contacting the sample with a plurality of target probe sets, wherein each target probe set is complementary to a target nucleic acid. This step is followed by contacting the sample with a first plurality of signal generating complexes (SGCs), each capable of hybridizing to a target probe set, and each comprising a plurality of spatially distributed label probes (LPs) comprising detectable labels. A first independent identifier is then obtained for each target nucleic acid, based on the number of distinct detectable labels present in each SGC of the first plurality of SGCs. In other embodiments, the method comprises first contacting the sample with a plurality of target probe sets, wherein each target probe set is complementary to a target nucleic acid. This step is followed by contacting the sample with a plurality of amplification complexes capable of hybridizing to at least one target probe set, wherein each amplification complex comprises a plurality of spatially distributed binding sites for label probes, and then contacting the sample with a first plurality of label probes comprising detectable labels, which hybridize to binding sites on the amplification complexes to generate a first plurality of signal generating complexes (SGCs). A first independent identifier is then obtained for each target nucleic acid, based on the number of distinct detectable labels present in each SGC of the first plurality of SGCs.

In one embodiment, anywhere from zero to ten different LPs bearing different detectable labels (e.g., from zero to eight, zero to six, or zero to four different detectable labels) can form combinations to generate each independent identifier. Examples are shown in Table 1a, where four different labels (L1, L2, L3 and L4) can generate 16 unique combinations for the first independent identifier. Further examples are shown in Table 1b, where five different labels (L1, L2, L3, L4, and L5) can generate 32 unique combinations for the first independent identifier. Use of six different labels would produce yet additional unique combinations. For example, fluorophores that can be used in combination to generate four, five, or six LPs include those with excitation maxima at or near at 488 nm, 550 nm, 594 nm, 650 nm, 700 nm, and/or 750 nm. The independent identifiers can each be easily identified, e.g., using an alphanumeric code, to generate a first independent identifier of each target. Specific methods and probe configurations for associating these different combinations of spatially distributed LPs with each target nucleic acid will be further discussed below.

TABLE 1a
Combinations of labels based on four distinct labels
to generate a first independent identifier of each
target (shown using an alphanumeric code).

| | Presence of labels (“X” present, “—” absent) | | | | Alphanumeric |
|---|---|---|---|---|---|
| Target | L1 | L2 | L3 | L4 | Code |
| 1 | — | — | — | — | 0 |
| 2 | X | — | — | — | 1 |
| 3 | — | X | — | — | 2 |
| 4 | — | — | X | — | 3 |
| 5 | — | — | — | X | 4 |
| 6 | X | X | — | — | 5 |
| 7 | X | — | X | — | 6 |
| 8 | X | — | — | X | 7 |
| 9 | — | X | X | — | 8 |
| 10 | — | X | — | X | 9 |
| 11 | — | — | X | X | A |
| 12 | X | X | X | — | B |
| 13 | X | — | X | X | C |
| 14 | X | X | — | X | D |
| 15 | — | X | X | X | E |
| 16 | X | X | X | X | F |

TABLE 1b
Combinations of labels based on four distinct labels
to generate a first independent identifier of each
target (shown using an alphanumeric code).

| | Presence of labels | | | | | Alpha- |
|---|---|---|---|---|---|---|
| | (“X” present, “—” absent) | | | | | numeric |
| Target | L1 | L2 | L3 | L4 | L5 | Code |
| 1 | — | — | — | — | — | 0 |
| 2 | X | — | — | — | — | 1 |
| 3 | — | X | — | — | — | 2 |
| 4 | — | — | X | — | — | 3 |
| 5 | — | — | — | X | — | 4 |
| 6 | — | — | — | — | X | 5 |
| 7 | X | X | — | — | — | 6 |
| 8 | X | — | X | — | — | 7 |
| 9 | X | — | — | X | — | 8 |
| 10 | X | — | — | — | X | 9 |
| 11 | — | X | X | — | — | A |
| 12 | — | X | — | X | — | B |
| 13 | — | X | — | — | X | C |
| 14 | — | — | X | X | — | D |
| 15 | — | — | X | — | X | E |
| 16 | — | — | — | X | X | F |
| 17 | X | X | X | — | — | G |
| 18 | X | X | — | X | — | H |
| 19 | X | X | — | — | X | I |
| 20 | X | — | X | X | — | J |
| 21 | X | — | X | — | X | K |
| 22 | X | — | — | X | X | L |
| 23 | — | X | X | X | — | M |
| 24 | — | X | X | — | X | N |
| 25 | — | X | — | X | X | O |
| 26 | — | — | X | X | X | P |
| 27 | X | X | X | X | — | Q |
| 28 | X | X | X | — | X | R |
| 29 | X | X | — | X | X | S |
| 30 | X | — | X | X | X | T |
| 31 | — | X | X | X | X | U |
| 32 | X | X | X | X | X | V |

Following generation of the first independent identifier, the signals generated by the detectable labels are removed from the sample, e.g., by removing the labels from the first plurality of SGCs, or by removing the first plurality of SGCs from the sample (as further discussed below). Next, the method comprises contacting the sample with a second plurality of SGCs, each capable of hybridizing to a target probe set, and each comprising a plurality of spatially distributed label probes (LPs) comprising detectable labels. Optionally, this step is preceded by contacting the sample with a second plurality of target probe sets, wherein each target probe set in the second plurality of target probe sets is complementary to a target nucleic acid. After contacting the sample with the second plurality of SGCs, a second independent identifier is obtained for each target nucleic acid, based on the number of distinct detectable labels present in each SGC of the second plurality of SGCs.

In some embodiments, the method further comprises identifying a target nucleic acid of the plurality of target nucleic acids based on a combination of the first and second independent identifiers (e.g., via generation of a “combined” identifier). Examples are shown in Table 2, where 16 target genes all have the same first independent identifier (alphanumeric code 1, in this example), and a second round of labeling provides a second independent identifier using four different labels (L1, L2, L3 and L4) that can generate 16 unique combinations for the second independent identifier.

TABLE 2
Combinations of labels based on four distinct labels to generate
a second independent identifier of each target nucleic acid.

| | First | | | | | Second | | | | | |
|---|---|---|---|---|---|---|---|---|---|---|---|
| | Independent Identifier | | | | | Independent Identifier | | | | | |
| | | | | | Alpha- | | | | | Alpha- | |
| Gene | | | | | numeric | | | | | numeric | Combined |
| Target | L1 | L2 | L3 | L4 | Code | L1 | L2 | L3 | L4 | Code | Identifier |
| 1 | X | — | — | — | 1 | — | — | — | — | 0 | 10 |
| 2 | X | — | — | — | 1 | X | — | — | — | 1 | 11 |
| 3 | X | — | — | — | 1 | — | X | — | — | 2 | 12 |
| 4 | X | — | — | — | 1 | — | — | X | — | 3 | 13 |
| 5 | X | — | — | — | 1 | — | — | — | X | 4 | 14 |
| 6 | X | — | — | — | 1 | X | X | — | — | 5 | 15 |
| 7 | X | — | — | — | 1 | X | — | X | — | 6 | 16 |
| 8 | X | — | — | — | 1 | X | — | — | X | 7 | 17 |
| 9 | X | — | — | — | 1 | — | X | X | — | 8 | 18 |
| 10 | X | — | — | — | 1 | — | X | — | X | 9 | 19 |
| 11 | X | — | — | — | 1 | — | — | X | X | A | 1A |
| 12 | X | — | — | — | 1 | X | X | X | — | B | 1B |
| 13 | X | — | — | — | 1 | X | — | X | X | C | 1C |
| 14 | X | — | — | — | 1 | X | X | — | X | D | 1D |
| 15 | X | — | — | — | 1 | — | X | X | X | E | 1E |
| 16 | X | — | — | — | 1 | X | X | X | X | F | 1F |

Notably, adding the second independent identifier doubles the total number of labels being used for each target nucleic acid. Accordingly, when 4 total labels are used in each round, and two cycles of labeling and detection are completed, a maximum of 255 targets can be detected (N=8; 2^{N}−1=255). (Note that the combined identifier 00 corresponds to no signal in either round of detection, so this code is not used.) When 5 total labels are used in each round, and two cycles of labeling and detection are completed, a maximum of 1023 targets can be detected (N=10; 2^{N}−1=1023).

In some embodiments, rather than identifying a target nucleic acid of the plurality of target nucleic acids based on a combination of the first and second independent identifiers, the method further comprises another round of labeling to generate a third independent identifier. Adding a third independent identifier further increases the number of independent targets that can be identified. When 4 total labels are used in each round, and three cycles of labeling and detection are completed, a total of 4095 targets can be detected (N=12; 2^{N}−1=4095). When 5 total labels are used in each round, and three cycles of labeling and detection are completed, a total of 32767 targets can be detected (N=15; 2^{N}−1=32767). Yet additional rounds of labeling can further increase the number of target nucleic acids that can be detected. Of course, fewer than the maximum total number of targets can be detected in any given assay.

a. Target Probe Sets

The disclosed methods comprise one or more steps of contacting a sample with a plurality of target probe sets, wherein each target probe set is complementary to a target nucleic acid. In the plurality of target probe sets, each individual set of target probes is specific for (i.e., specifically hybridizes to) each target nucleic acid in the plurality of target nucleic acids being detected in the method. Each target probe set comprises one or more pairs of individual target probes.

As used herein, a “target probe” refers to a polynucleotide that is capable of hybridizing to a target (e.g., a target nucleic acid) and capturing or binding a signal-generating complex (SGC) component to that target. For example, the target probe can hybridize to an amplifier, a pre-amplifier or a pre-pre-amplifier in an SGC. The target probe thus includes a first polynucleotide sequence that is complementary to a polynucleotide sequence of the target nucleic acid, and a second polynucleotide sequence that is complementary to a polynucleotide sequence of a component of a SGC (amplifier, pre-amplifier, pre-pre-amplifier, or the like). The target probe is generally single stranded so that the complementary sequence is available to hybridize with a corresponding target, amplifier, pre-amplifier, pre-pre-amplifier, or the like.

Each target probe comprises a target (T) section and a label (L) section, wherein the T section is a nucleic acid sequence complementary to a section on the target nucleic acid and the L section is a nucleic acid sequence complementary to a section on the nucleic acid component of the SGC. Within each target probe pair, the T sections are complementary to non-overlapping regions of the target nucleic acid, and the L sections are complementary to non-overlapping regions of the nucleic acid component(s) of the SGC.

In each target probe pair, the two target probes can have the same directionality, or opposite directionalities. For example, in some embodiments, T section of each target probe in the target probe pair is 3′ of its L section. In some embodiments, the T section of each target probe in the target probe pair is 5′ of its L section. In some embodiments, the T section of one target probe in the target probe pair is 3′ of its L section, and the T section of the other target probe in the target probe pair is 5′ of its L section.

In some embodiments, each target probe comprises a non-binding portion separating its T section from its L section (e.g., a spacer or linker region). In some embodiments, this non-binding portion does not bind to either the target nucleic acid or the nucleic acid component of the SGC, but is useful in forming the SGC. In some embodiments, this non-binding region can form the unpaired loop section of a hairpin loop. In other embodiments, none of the target probes contains a non-binding portion.

In some embodiments, one pair of target probes is used to detect each target nucleic acid. In other embodiments, two or more pairs of target probes are used to detect each target nucleic acid. In such a case, the pairs of target probes in the target probe set specific for a particular target nucleic acid bind to different and non-overlapping sequences of the target nucleic acid. For example, when two or more pairs of target probes specifically hybridize to the same target nucleic acid, the molecule that binds to the target probe pairs (i.e., an amplifier, a pre-amplifier, or a pre-pre-amplifier) generally are the same for target probe pairs in the same target probe set. Thus, the target probe pairs that bind to the same target nucleic acid can be designed to include the same binding site for the molecule in the SGC that binds to the target probe pairs (e.g., a pre-amplifier or pre-pre-amplifier). The use of multiple target probe pairs to detect a target nucleic acid can provide for a higher signal associated with the assembly of multiple SGCs on the same target nucleic acid. In some embodiments, the number of target probe pairs used for binding to the same target nucleic acid are in the range of 1-10, 1-20, 1-30, 1-40, 1-50, 1-60, 1-70, 1-80, 1-90, 1-100, 1-110, 1-120, 1-130, 1-140, 1-150, 1-160, 1-170, 1-180, 1-190, or 1-200 pairs per target, or larger numbers of pairs, or any integer number of pairs in between, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, and the like.

Each target probe pair can be designed to bind to immediately adjacent segments of the target nucleic acid. or on segments that have one to a number of bases between the target probe binding sites of the target probe pair. Generally, target probe pairs are designed for binding to the target nucleic acid such that there are generally between 0 to 500 bases between the binding sites on the target nucleic acid, for example, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 420, 440, 460, 480, or 500 bases, or any integer length in between. In particular embodiments, the binding sites for the pair of target probes are between 0 to 100, 0 to 200, or 0 to 300 bases, or any integer length in between. In the case where more than one target probe pair is used in a target probe set to bind to the same target, and where there is a gap in the binding sites between a pair of target probes, it is understood that the binding sites of different target probe pairs do not overlap. In the case of detecting double stranded nucleic acids, such as DNA, some overlap between different target probe pairs can occur, so long as the target probe pairs are able to concurrently bind to the respective binding sites of the double stranded target nucleic acid.

b. Signal Generating Complexes and Labels

In some embodiments, the disclosed methods comprise one or more steps of contacting a sample with a plurality of signal generating complexes (SGCs) capable of hybridizing to at least one target probe set, wherein each SGC comprises a plurality of spatially distributed label probes comprising detectable labels. In some embodiments, the disclosed methods comprise one or more steps of contacting a sample with a plurality of amplification complexes capable of hybridizing to at least one target probe set, wherein each amplification complex comprises a plurality of spatially distributed binding sites for label probes; subsequently, the sample is contacted with a plurality of label probes comprising detectable labels, which hybridize to binding sites on the amplification complexes to generate a plurality of SGCs. Various configurations of amplification complexes and SGCs can be employed in the above methods, to allow for binding of 0, 1, 2, 3, or 4 different labels to each target nucleic acid, to generate each independent identifier for the target nucleic acid (e.g., a first independent identifier, a second independent identifier, a third independent identifier, etc.)

As used herein, the term “label probe” refers to an entity that binds to a target molecule, directly or indirectly, generally indirectly, and allows the target to be detected. A label probe (or “LP”) contains a nucleic acid binding portion that is typically a single stranded polynucleotide or oligonucleotide that comprises one or more labels which directly or indirectly provides a detectable signal. The label can be covalently attached to the polynucleotide, or the polynucleotide can be configured to bind to the label. For example, a biotinylated polynucleotide can bind a streptavidin-associated label. Generally, the label probe can hybridize to a nucleic acid that is in turn hybridized to the target nucleic acid or to one or more other nucleic acids that are hybridized to the target nucleic acid. Thus, the label probe can comprise at least one polynucleotide sequence that is complementary to a polynucleotide sequence in an amplifier, pre-amplifier, or pre-pre-amplifier in an amplification complex or SGC.

In some embodiments, the amplification complexes and SGCs provided herein comprise components such an amplifier, a pre-amplifier, and/or a pre-pre-amplifier. As used herein, an “amplifier” is a molecule, typically a polynucleotide, that is capable of hybridizing to multiple label probes. The amplifier hybridizes to multiple identical label probes, or multiple different label probes. The amplifier can also hybridize to a target nucleic acid, to at least one target probe of a pair of target probes, to both target probes of a pair of target probes, or to nucleic acid bound to a target probe such as a pre-amplifier or pre-pre-amplifier. For example, the amplifier can hybridize to at least one target probe and to a plurality of label probes, or to a pre-amplifier and a plurality of label probes. The amplifier can be, for example, a linear, forked, comb-like, or branched nucleic acid. As described herein for all polynucleotides, the amplifier can include modified nucleotides and/or nonstandard internucleotide linkages as well as standard deoxyribonucleotides, ribonucleotides, and/or phosphodiester bonds. Suitable amplifiers are described, for example, in U.S. Pat. Nos. 5,635,352, 5,124,246, 5,710,264, 5,849,481, and 7,709,198 and U.S. publications 2008/0038725 and 2009/0081688, each of which is incorporated by reference.

As used herein, a “pre-amplifier” is a molecule, typically a polynucleotide, that serves as an intermediate binding component between one or more target probes and one or more amplifiers. Typically, the pre-amplifier hybridizes simultaneously to one or more target probes and to a plurality of amplifiers. Exemplary pre-amplifiers are described, for example, in U.S. Pat. Nos. 5,635,352, 5,681,697 and 7,709,198 and U.S. publications 2008/0038725, 2009/0081688 and 2017/0101672, each of which is incorporated by reference.

As used herein, a “pre-pre-amplifier” is a molecule, typically a polynucleotide, that serves as an intermediate binding component between one or more target probes and one or more pre-amplifiers. Typically, the pre-pre-amplifier hybridizes simultaneously to one or more target probes and to a plurality of pre-amplifiers. Exemplary pre-pre-amplifiers are described, for example, in U.S. Pat. No. 11,078,528, which is incorporated by reference.

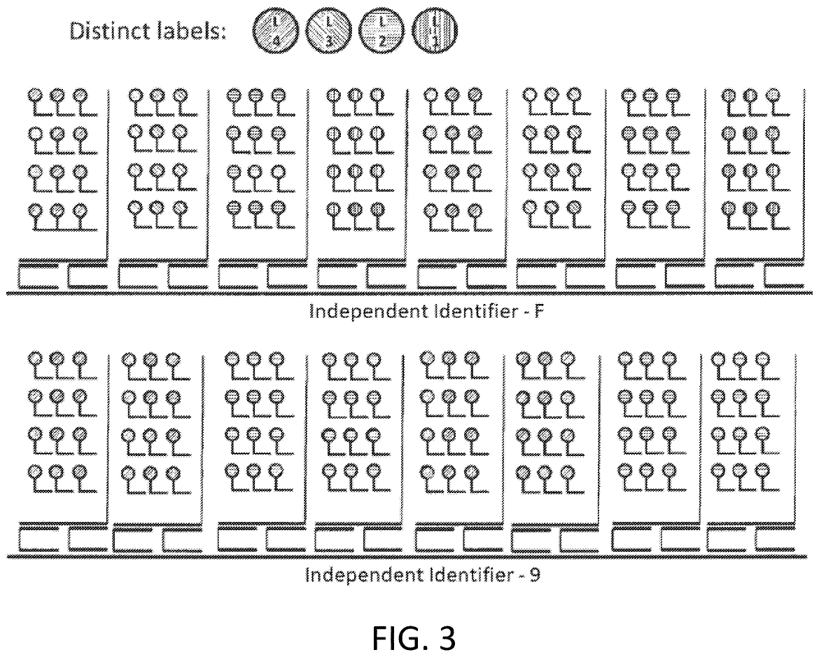

Each SGC comprises a plurality of spatially distributed label probes comprising detectable labels. The labeling scheme can be implemented in different ways. For example, in some embodiments, the labeling scheme can be employed at the SGC level. As shown in FIG. 3, for generation of an independent identifier with N unique labels, N unique label-specific SGCs can be made. Each SGC carries the same LP with a specific label. Components of each SGC (such as PAs, APs, LPs, etc.) are uniquely associated with the SGC. They are specially designed to hybridize to each other to assemble the SGC but cannot cross-hybridize to any components of any other SGC. In the exemplary embodiment shown in FIG. 3, two target nucleic acids are shown bound to the respective SGCs. For one target nucleic acid (upper target in FIG. 3), the SGCs for the target comprise four labels (L1, L2, L3, and L4). For a second target (lower target in FIG. 3), the SGCs for the target nucleic acid comprise two labels (L2 and L4). When the target sequence is sufficiently long, many TP pairs can be designed to bind specifically to the target. Each TP pair can be coupled to a selected SGC through its L sections (see FIG. 1). Many SGCs can be captured to each target in this way. An independent identifier of each target can be generated through the unique combination of SGCs. An advantage of SGC-level implementation is that only a relatively small number of different SGCs need to be developed. Since it is essential that components of each SGC do not cross-hybridize to other SGCs, the amount of work involved in developing a large number of SGCs is substantial. With this configuration, the length of target sequence has to be sufficiently long to accommodate many SGCs. Some SGCs may not be assembled successfully on the target due to accessibility of the target sequence. This may lead to miscoding, which can be addressed as described below.

Accordingly, in one embodiment, the plurality of SGCs comprise: (a) a set of pre-amplifiers, wherein the pre-amplifier set comprises one or more subsets of pre-amplifiers, wherein the one or more pre-amplifier subsets comprise a pre-amplifier specific for each of the target probe pairs in the plurality of target probe sets, wherein each pre-amplifier comprises binding sites for the pair of target probes of one of the target probe sets and a plurality of binding sites for an amplifier; (b) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe; and (c) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets; wherein the one or more label probe subsets in each probe subset specific for a target nucleic acid comprise at least one label or a combination of labels that is different for each probe subset (see FIG. 3).

The labeling scheme can also be implemented at the component level within an SGC (i.e., sub-SGC level). Such a system comprises N different, label specific LPs and 2N−1 unique, target-specific SGCs. Each LP has a segment designed to hybridize to a label probe anchor (LA) on an AP molecule (i.e., the LA being the binding site on the amplifier for the label probe) in the SGC. A mixture of different LAs are designed and made to bind a set of pre-determined, different LPs, generating a unique combination of detectable labels that are used to identify the target. The sub-SGC level implementation can be advantageous when the target sequence is shorter. In addition, the probability of miscoding is reduced, as discussed below. This approach can be adopted to detect a single base variant or a unique junction in the target sequence (see, for example, WO 2017/066211). However, a comparatively large number of unique SGCs (2N−1) need to be developed.

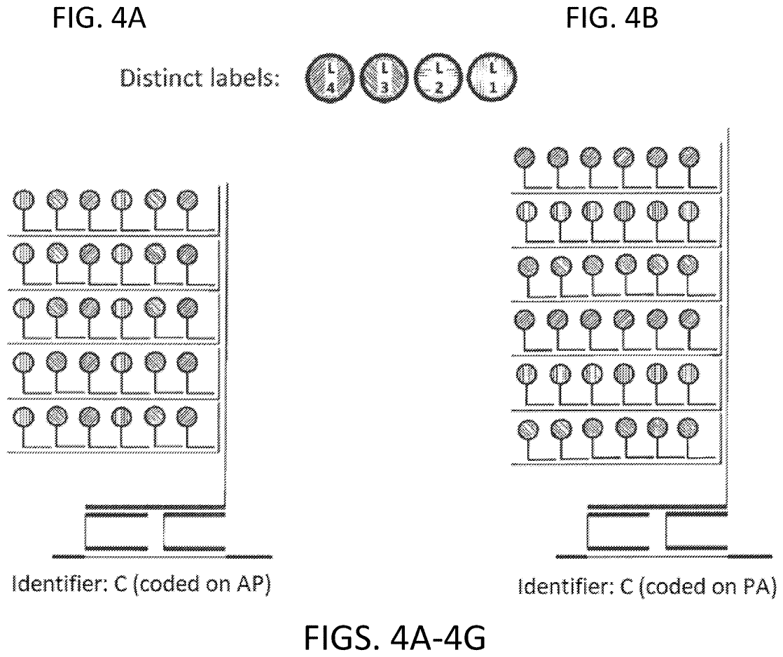

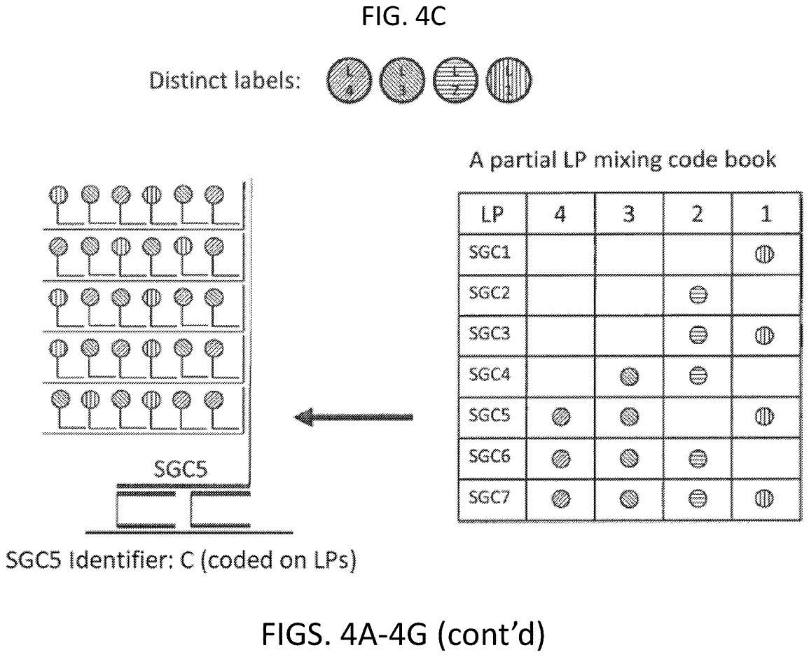

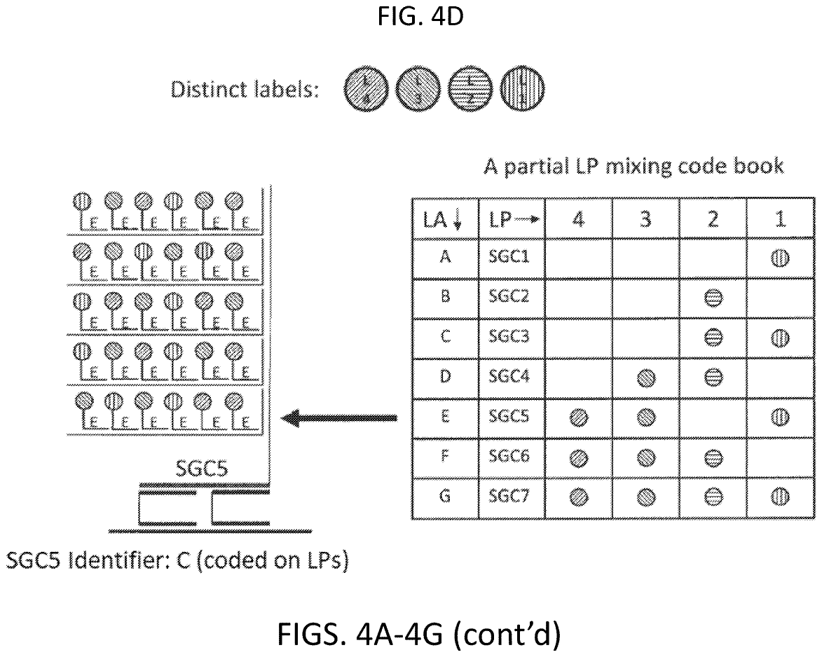

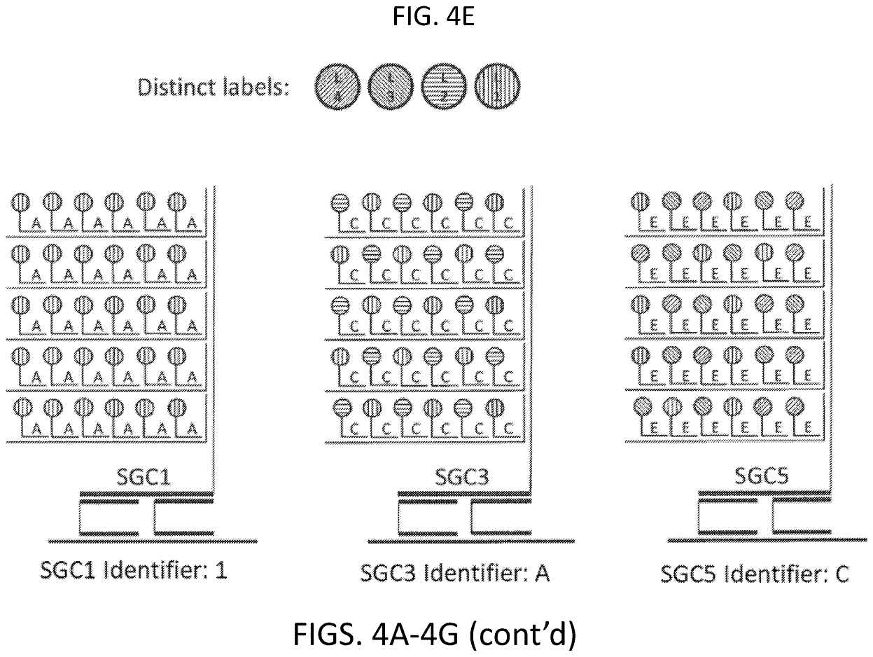

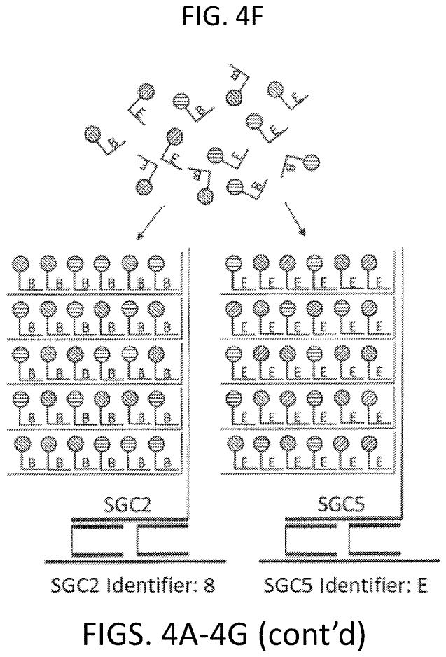

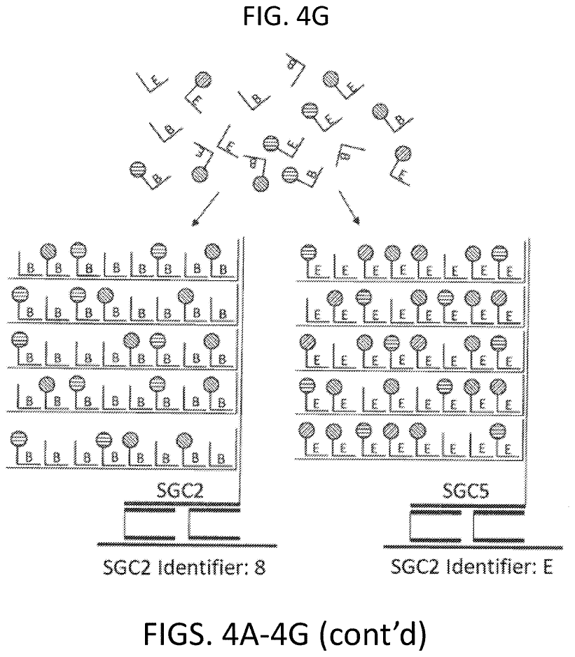

FIG. 4A illustrates one embodiment of sub-SGC implementation, where the above mentioned SGC ID code is implemented on the AP molecule. As shown in FIG. 4A, an AP has one region designed to bind to the amplifier anchor (AA) on the PA (i.e., the AA being the binding site on the pre-amplifier for the amplifier) and another region comprising multiple segments of LAs (i.e., the binding sites on the amplifier for the label probes). In previously described methods, the amplifier used repeats of the same LA, that is, the amplifier had a plurality of binding sites for the same label probe. An embodiment using repeats of the same LA for binding a plurality of the same LPs to the amplifier is shown in FIG. 3. In the embodiment shown in FIG. 4A, however, a mixture of different LAs are designed according to the unique identifier of the SGC. For example, if the independent identifier of the SGC includes three separate labels (e.g., L1, L3, and L4, as shown in FIG. 4A), then an equal number of LAs for LPs with L1, L3, and L4 are made on the AP molecule, which will bind a designed number of desired LP to generate the independent identifier.

Accordingly, some embodiments, the plurality of SGCs comprise: (a) a set of pre-amplifiers, wherein the pre-amplifier set comprises a plurality of pre-amplifiers, wherein the pre-amplifiers comprise binding sites for the pairs of target probes and a plurality of binding sites for an amplifier; (b) a set of amplifiers, wherein the amplifier set comprises a plurality of amplifiers, wherein the amplifiers comprise a binding site for the pre-amplifiers and a plurality of binding sites for a label probe or two or more distinct label probes; and (c) a set of label probes, wherein the label probe set comprises a label probe or two or more distinct label probes, wherein each label probe comprises a label and a binding site for the amplifiers, wherein the labels in each distinct label probe are distinguishable between the distinct label probes; wherein the amplifier in each probe subset specific for a target nucleic acid comprises a binding site for a label or a combination of two or more distinct labels that is different for each probe subset (see FIG. 4A).

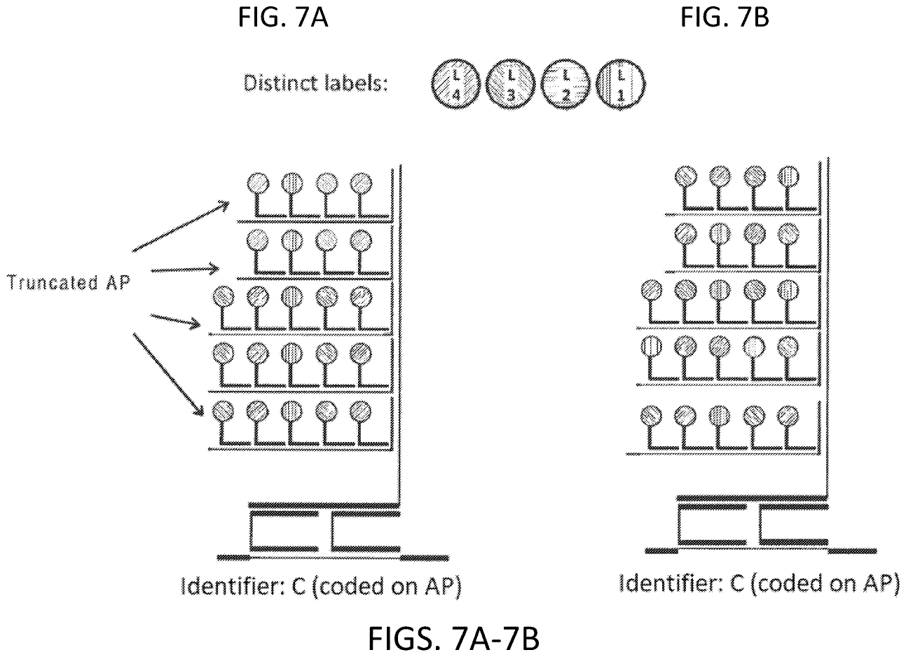

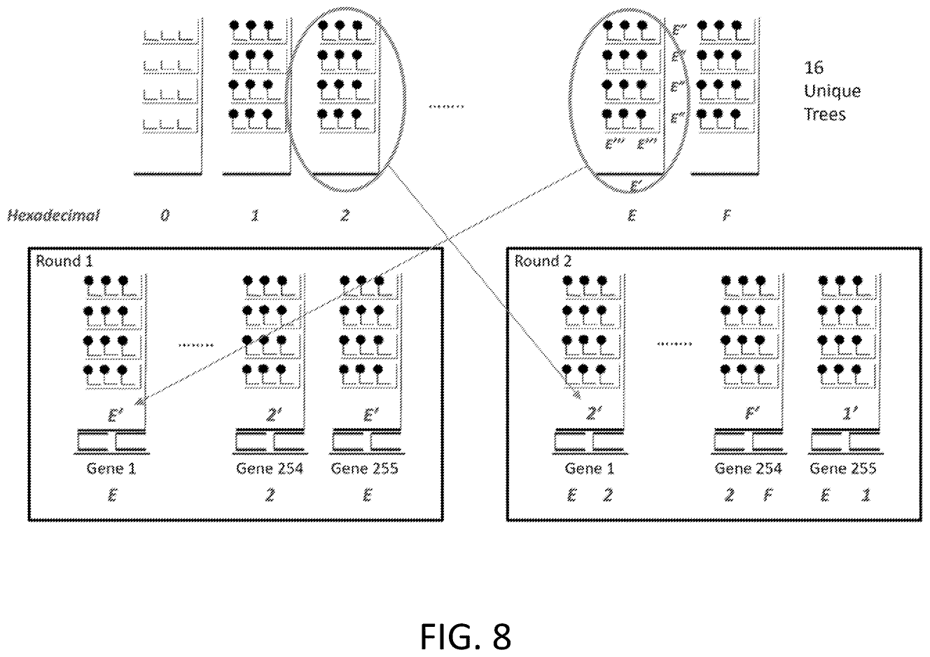

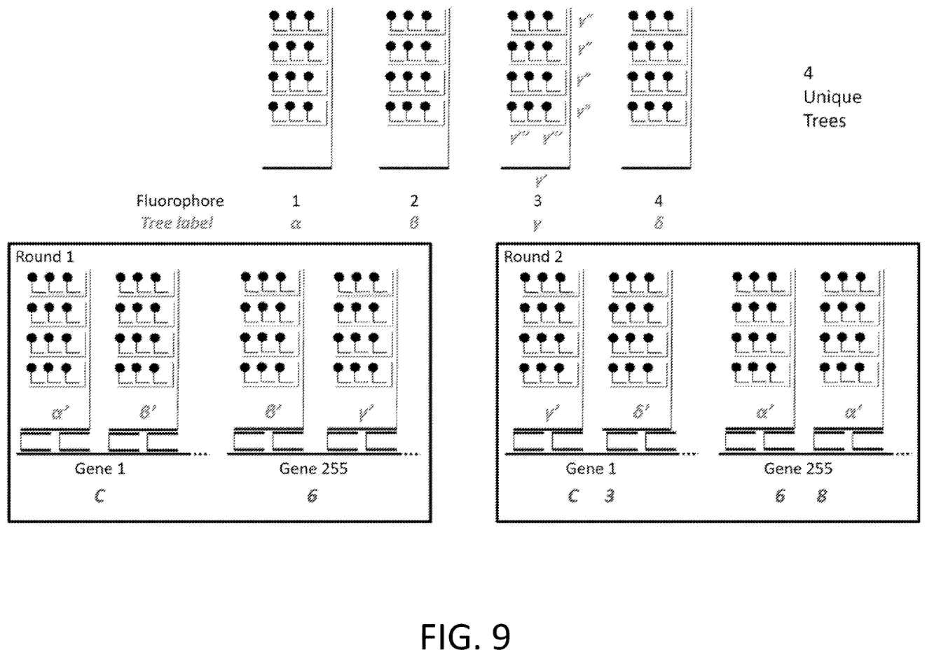

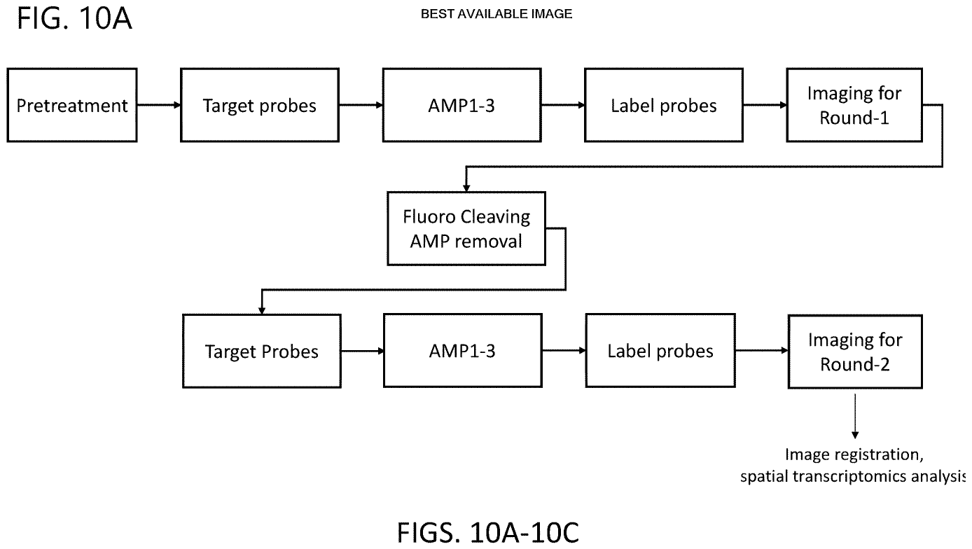

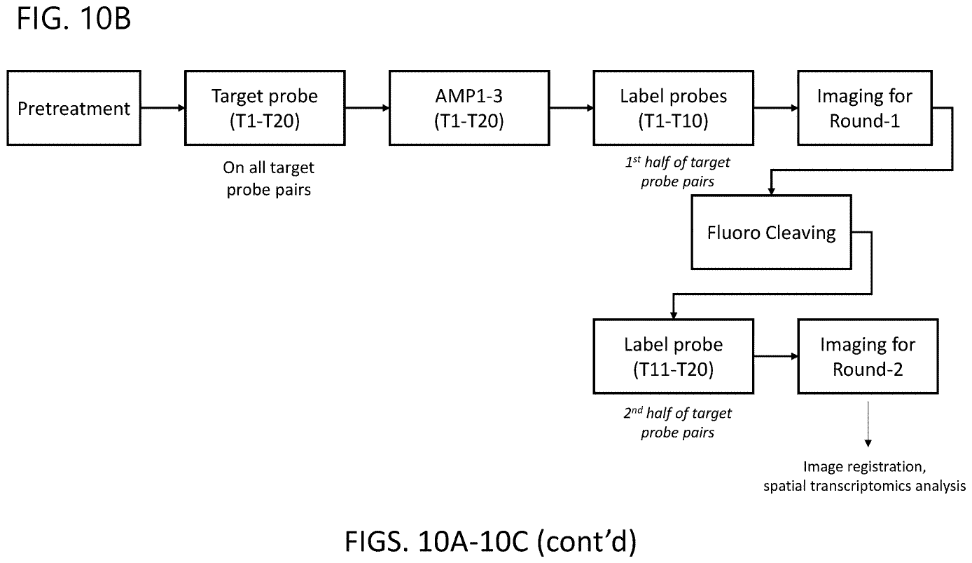

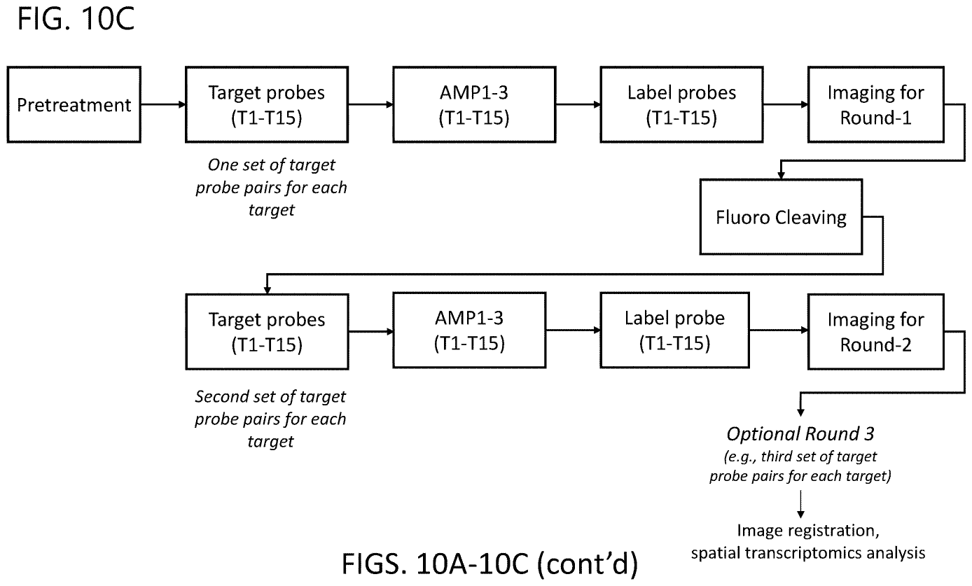

In one embodiment of such a method, the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier (see FIG. 7B). This embodiment can be used to reduce miscoding, as described below in more detail.

FIG. 4B illustrates another embodiment of sub-SGC implementation, where the SGC ID code is implemented on the PA molecule. As shown in FIG. 4B, N “pure” AP molecules are made, each carrying the same LA for the same type of LP. A PA molecule has one region designed to bind to the L-section of the target probe (i.e., the segment of the TP that binds to the pre-amplifier; see FIG. 1A) of a target probe pair, and another region comprising multiple segments of AAs (amplifier anchors, i.e., the segments on the pre-amplifier that bind to the amplifiers). In previously disclosed methods, these are repeats of the same AA. In the embodiment shown in FIG. 1B, however, a mixture of different AAs are designed according to the unique identification code of the SGC. For example, if the independent identifier of the SGC is based on two labels, then equal number of AAs for APs carrying the two LPs are made on the PA molecule, which will bind a designed number of desired LPs to generate the identifier in the assay. In the exemplary embodiment shown in FIG. 4B, the code for the SGC shown uses three labels (L1, L3, and L4).

Accordingly, in one embodiment, the plurality of SGCs comprise: (a) a set of pre-amplifiers, wherein the pre-amplifier set comprises a plurality of pre-amplifiers, wherein the pre-amplifiers comprise binding sites for the pairs of target probes and a plurality of binding sites for amplifiers; (b) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe; and (c) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes; wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset (see FIG. 4B).

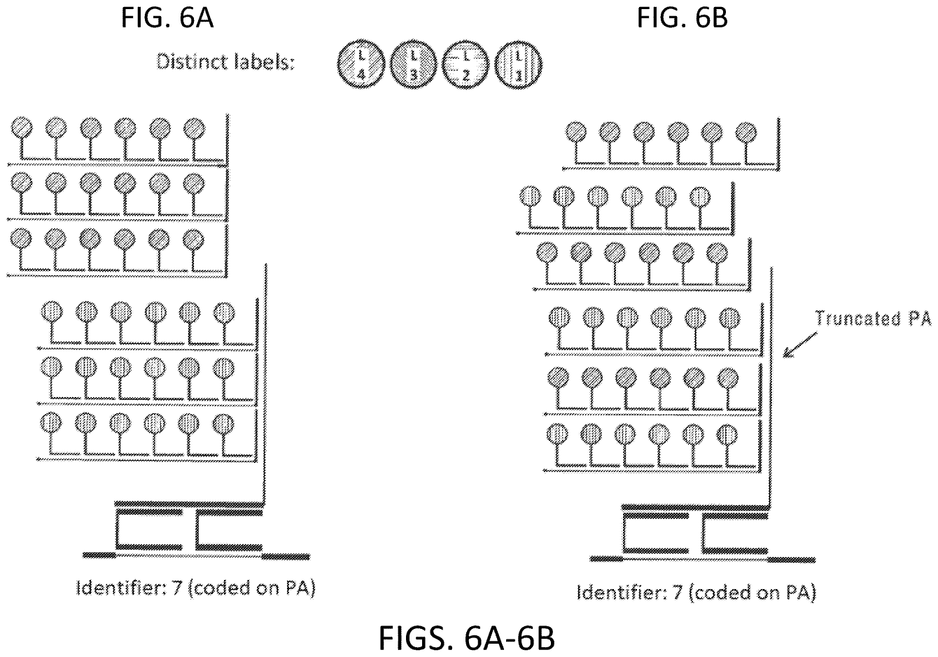

In one embodiment of such a method, the plurality of amplifiers comprise two or more distinct amplifiers, and wherein the binding sites on the pre-amplifier for the distinct amplifiers are intermingled (see FIG. 6B). As described below in more detail, this embodiment can be used to reduce miscoding.

FIG. 4C illustrates another embodiment of sub-SGC implementation, where the independent identifier is implemented on the LP molecule. In previously described methods, the LP sequences bound to a single SGC all carry the same label. The embodiment of FIG. 3 also utilizes the same labels on a given SGC. In the embodiment depicted in FIG. 4C, LP molecules binding to the same SGC can be a mixture of LPs each conjugated to a different label according to a predefined code book. For example, as shown in FIG. 4C, SGC5 LPs are a mixture of LPs conjugated to three different labels. An advantage of this embodiment is that the “coloring” of the SGC complex by the LPs will be completely randomized, which can further help to reduce coding errors. Since the SGC identifiers are not hard coded in the SGCs, this scheme provides the flexibility to assign different identifiers to different SGCs in different assay configurations simply by devising a different code book on the fly. In addition, the mixing of LPs can be made in unequal amounts to normalize the labeling intensities across the N labels, which again can help in reducing encoding/decoding errors. The disadvantage of this embodiment is that M^{N}−1 LP and LA sequences will be required to uniquely encode and decode each SGC. In comparison, in the embodiment shown in FIG. 4A, only N unique LPs and LAs are required.

As shown in FIG. 4C, the SGC ID code is implemented on the LP molecule. In this embodiment, LP molecules binding to the same SGC can be mixtures of LPs each conjugated to a different label according to a predefined code book. For example, as shown in FIG. 4C, SGC5 LPs are a mixture of LPs conjugated to three different labels, corresponding to labels L1, L3, and L4. An advantage of this embodiment is that the “coloring” of the SGC complex by the LPs will be completely randomized, which can further help to reduce coding errors. A partial LP mixing code book is shown on the right of FIG. 4C, with 7 different exemplary SGC codes shown using 4 labels.

FIG. 4D shows an embodiment of FIG. 4C in more detail. As depicted in FIG. 4D, for each SGC, a specific label anchor (LA, the binding site on the amplifier for the label probe) is assigned so that each SGC for a particular target nucleic acid has a plurality of the same LAs on the amplifier. The level at which combinatorial labeling can be provided is with the label probes (LPs). In this case, SGC5 is illustrated showing that the amplifiers comprise a plurality of identical LAs, labeled “E.” As shown in FIG. 4D, SGC5 is coded with 3 distinct label probes (L1, L3, and L4), all of which have the same binding site for the plurality of “E” LAs on the corresponding amplifiers. Therefore, all three label probes are bound to the amplifiers of SGC5.

FIG. 4E shows an embodiment of FIG. 4C in more detail. The SGC5 of FIG. 4D is shown bound to its respective target nucleic acid, with the label probes having an “E” binding site bound to the respective “E” LAs of the SGC5 amplifiers (as in FIG. 4D). Also shown are two additional exemplary SGCs bound to their respective target nucleic acids. SGC1, coded as shown in FIG. 4D, comprises a plurality of identical LAs, labeled “A.” SGC1 is coded with one label probe (L1), which has the binding site for the plurality of “A” LAs of the SGC1 amplifiers. Therefore, the label probe “L 1” is bound to the amplifiers of SGC1, thereby labeling the SGC1 target nucleic acid. SGC3, coded as shown in FIG. 4D, comprises a plurality of identical LAs, labeled “C.” SGC3 is coded with 2 probes corresponding to 2 distinct label probes (L1 and L2), both of which have the same binding site for the plurality of “C” LAs on the corresponding SGC3 amplifiers. Therefore, both label probes are bound to the amplifiers of SGC3, thereby labeling the SGC3 target nucleic acid.

FIG. 4F shows an embodiment of FIG. 4C in more detail. FIG. 4F illustrates that, once an SGC for a particular target nucleic acid has been designed, the actual coding for the target nucleic acid can be readily modified simply by changing the labels on the label probes that bind to the amplifiers of a particular SGC. For example, in FIG. 4D, SGC2 comprises amplifiers with “B” LAs and is coded using L2. In FIG. 4F, the same SGC assembly can be used with respect to the target probes, pre-amplifier, and amplifiers with “B” LAs, but instead of using “B” LA-binding label probes with only L2 as in FIG. 4B, “B” LA-binding label probes can be used that have a mixture of L2 and L3 such that SGC2 is now coded with both labels. Thus, labels L2 and L3 are bound to “B” LAs on the SGC2 amplifiers. Similarly, SGC5 comprising amplifiers with “E” LAs is now coded in FIG. 4F by using label probes with “E” LA-binding label probes that have a mixture of labels L2, L3, and L4, instead of labels L1, L3, and L4 as shown in FIG. 4D.

As described herein, in some embodiments, SGCs for different target nucleic acids will have different numbers of labels in the code. In this situation and in the case where the number of LAs on the respective SGCs is the same, if the number of label probes are added to the SGCs, the SGC coded with only one label will have a higher number of bound labels than the number of corresponding probes bound to an SGC coded with four labels; for example an SGC coded with only L1, L1 can bind to all of the sites on one of the SGCs, but for an SGC coded with L1, L2, L3, and L4, L1 can only bind to ¼ of the sites on the SGC. Accordingly, in some embodiments, it can be desirable to normalize the amount of label bound to different SGCs coded by different numbers of distinct labels.

In an exemplary embodiment as shown in FIG. 4G, two target nucleic acids are shown with two bound SGCs, SGC2 and SGC5. SGC2 is coded with L2 and L3, and SGC5 is coded with L2, L3, and L4. In this case, where all of the label probes bind to the respective LAs, “B” LAs in the case of SGC2 and “E” LAs in the case of SGC5, and assuming that the SGC2 and SGC5 have approximately the same number of LAs in the respective SGCs, the number of respective labels that can bind to SGC2 will be higher than the number of respective labels that bind to SGC5 (i.e., the 2 distinct labels for SGC2 (L2 and L3) and the 3 distinct labels for SGC5 (L2, L3, and L4) will be bound to the same number of sites, resulting in a higher number of L2 and L3 being bound to SGC2 than SGC5 since some of the SGC5 sites are occupied by L4). If desired, the number of labels (and therefore intensity of signal) can be normalized by including “blank” label probes, i.e., probes having a binding site for the respective LAs (in this case “B” for SGC2 and “E” for SGC5) but without a label. For example, if it is desired to compare SGC2 and SGC5 with equal intensity signals for the respective labels, ⅓ “blank” label probes can be included with the mixture of “B” LA-specific probes so that the intensity of L2 and L3 will be the same on both SGCs (i.e., ⅓ of SGC2 occupied by “blank” label probes and ⅓ of SGC5 occupied by L4). In another example, if a assay is being performed in which some SGCs include 4 labels, then the assay can be performed so that the same proportion of “blank” label probes are included in the label probe sets using less than 4 labels, for example, ½ “blank” label probes can be included with the SG2-specific label probes coded by 2 distinct labels, and ¼ “blank” label probes can be included with the SGC5-specific label probes coded by 3 distinct labels, so that the amount of each distinct label probe (L1, L2, L3, and L4) bound to the respective SGCs is the same on each SGC. Such “blank” label probes can also be used in combination with distinct label probes to provide for a desired proportion of respective labels, such as a desired ratio of label probes on an SGC.

In one embodiment, the plurality of SGCs comprises: (a) a set of pre-amplifiers, wherein the pre-amplifier set comprises a plurality of pre-amplifiers, wherein the pre-amplifiers comprise binding sites for the pairs of target probes and a plurality of binding sites for an amplifier; (b) a set of amplifiers, wherein the amplifier set comprises a plurality of amplifiers, wherein the amplifiers comprise a binding site for the pre-amplifiers and a plurality of identical binding sites for a label probe; and (c) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes; wherein the amplifier in each probe subset specific for a target nucleic acid comprises a binding site for a label or a combination of two or more distinct labels that is different for each probe subset (see FIG. 4C).

Similar approaches can be implemented on other components of the SGC and the two embodiments shown in FIG. 4A and FIG. 4B can be used in combination to ensure the pre-determined mixture of LPs can be assembled onto the SGC to generate the independent identifier.

In some embodiments, the methods of the disclosure can be modified to reduce miscoding. Miscoding can occur when the signal from a type of LP designed to be present with a particular target is undetectable, or background noise is misinterpreted as a signal from a particular LP. Errant positive signals can be largely eliminated by setting up an appropriate threshold. Signal level from the area surrounding the image dot as well as the global background level can be used to reference the background. Methods for reducing errant undetected signal-type miscoding are described herein.

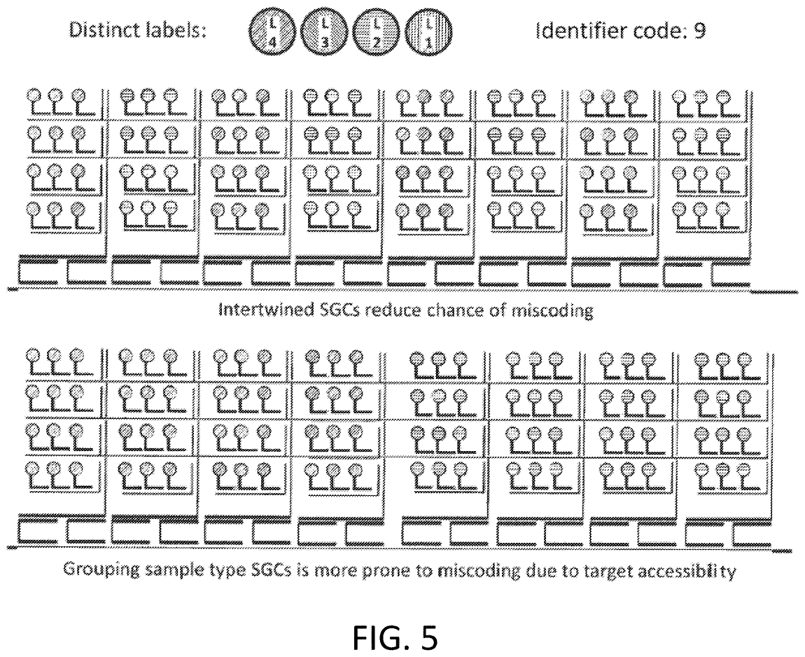

When the SGC level code implementation method described previously is used, errant “null”-type miscoding can occur when SGCs of a certain LP type are not attached to the target due to limited TP accessibility to the target or target degradation. It is therefore important that each label-specific SGC has many copies in the set designed to bind to the same target so that there are statistically many chances for each needed LPs to be present in the detected signal. This means that the target sequence has to be very long in order to reduce the miscoding rate. For example, assuming four different LPs are used, 20 copies of each LP specific SGCs can be needed to ensure reliable detection and each SGC binds to a TP set occupying 50 nucleotides (50 nt), so the total target length in this case has to be 4×20×50=4000 nucleotides. Also, it may be advantageous to intertwine different SGCs along the target, as shown in FIG. 5. If different SGC types are positioned apart in separate groups, as depicted in FIG. 5, lower panel, a certain section of the target may be blocked or masked, thereby preventing attachment of one specific SGC type, which will result in miscoding. Designing the target probes in the SGC of the same type, when more than one type of SGC is used, to bind to the target nucleic acid at sites that are intermingled or intertwined, as shown in FIG. 5, upper panel, reduces the chance of miscoding.

From the point of view of reducing miscoding, there are advantages to implementing the ID code at the sub-SGC level because, once a TP set is successfully hybridized to the target, there are many more chances for different LPs to successfully attach to the SGC without bias. Arranging different labels into alternating positions is still an important strategy to reduce the chance of miscoding. As shown in FIG. 6A, this particular SGC is miscoded; only L1 is bound, when the intent was for L1 and L4 to be bound. In this case the miscoding occurred because the PA is truncated, which can occur during manufacturing of the PA. The same truncation, however, will not cause miscoding if the labels are intertwined or intermingled on the PA, if the embodiment shown in FIG. 4B is implemented as shown in FIG. 6B. A similar strategy can be used in a configuration with AP level coding, as illustrated in FIG. 4A. An additional method to minimize the potential miscoding caused by truncation is to randomize the position of different labels on the AP or PA that encodes the target specific code, as illustrated in FIG. 7, in which the multiplexing channel ID is encoded on the AP molecule. In FIG. 7A, different label probes are positioned on each AP in exactly the same way, that is, each amplifier is the same. Truncation of the AP, for example, during manufacturing, may cause substantial reduction in certain label compared to others. This imbalance increases the chance of miscoding. In the most severe case, truncation may cause the loss of all copies of one certain label, leading to an outright miscode. In FIG. 7B, locations of different labels on the AP are purposefully randomized. Truncation therefore does not cause a large bias in the numbers and types of labels in the SGC. The APs are provided as a plurality of amplifiers, where a mix of non-identical amplifiers is included, where the position of LAs for specific label probes are distributed differently and can be randomized on the non-identical amplifiers. Randomization of different labels in the SGCs can be achieved by using one or combinations of the embodiments described herein.

When miscoding occurs because a label that should be present is absent, the target may be mis-identified as another target with fewer labels in its ID code. In most situations, the probability of miscoding is low (e.g., <5%). When the quantities of targets are in a similar level, such miscoding does not significantly impact the results. Miscoding can have a significant impact if one target is present at a significantly higher quantity than the other target that it miscoded into (i.e., one target is miscoded to be misread as another target due to differences in amounts of the two targets). Therefore, one important method to reduce the impact of miscoding is to assign ID codes with fewer labels to higher quantity targets if the relative quantities of the targets are known. For example, in Table 1a, the independent identifier of gene targets 2-5 are each generated using a single label, and in Table 1b, the independent identifier in gene targets 2-6 are each generated using a single label. These “codes” can be reserved for targets with the highest quantities because if any miscoding occurs due to the erroneous absence of labeling, they will not be mis-interpreted into the signals for other targets.

Many error detection schemes used in the digital communication field can be adopted to detect miscoding. For example, a parity check can ensure accurate data transmission during communication. A parity bit is appended to the original data bits to make an even or odd number of total data bits. For example, the signal from one of the N Labels can be used as a “parity check” bit (L1 in the example codebook in Table 3). The bit will be made “X” (present) or “-” (absent) to make the total number of labels in the N label system odd (i.e., odd parity check) or even (i.e., even parity check). Depending on the result of the parity check, the detected target may or may not be counted. This parity check scheme can detect single or odd number of bit errors but cannot detect double or even number bit errors. This can substantially reduce the probability of miscoding. For example, if the chance of single bit error is 5%, the chance of double bit error is theoretically 0.25%. The price for using such a parity check is that the number of multiplexing channels is reduced to 2^{N}−1 or 2^{N}−1 if an even or odd parity check scheme is adopted, respectively (e.g., 7 or 8 SGC codes in Table 3 compared to 15 SGC codes in Table 1a). A code book can be designed to include an odd (or even) number of labels. In the example shown in Table 1a, for an odd number, the allowed channels from Table 1a would be those for gene targets 2, 3, 4, 5, 12, 13, 14, and 15. If one of the other channels is detected, then it must be an error.

TABLE 3
Labeling Scheme with Parity Check.

| | | Presence of labels | | | | |
|---|---|---|---|---|---|---|
| | | (“X” present, “—” absent) | | | | |
| | | | | | L1 = Even | L1 = Odd |
| | | L4 | L3 | L2 | Parity Bit | Parity Bit |
| Gene | 1 | — | — | X | X | — |
| Target | 2 | — | X | — | X | — |
| | 3 | X | — | — | X | — |
| | 4 | — | X | X | — | X |
| | 5 | X | — | X | — | X |
| | 6 | X | X | — | — | X |
| | 7 | X | X | X | X | — |
| | 8 | — | — | | — | X |

As described herein, a specific target nucleic acid can be labeled with more than one distinct label. In such cases, a single “dot” will be comprised of two or more distinct labels. A dot comprising more than one label can be deconvoluted to identify the individual labels in the dot using well known methods. Such well known methods include the Richardson-Lucy deconvolution algorithm (see Example I) as described previously (Biggs et al., Applied Optics, Vol. 36, No. 8, (1997); Hanisch et al., “Deconvolutions of Hubble Space Telescope Images and Spectra, Deconvolution of Images and Spectra,” Ed. P. A. Jansson, 2nd ed., Academic Press CA, (1997)). Other methods for deconvolution include, but are not limited to, Wiener deconvolution, regularized filter deconvolution, and the like (Gonzalez et al., “Digital Image Processing,” Addison-Wesley Publishing Company, Inc. (1992)).

The embodiments described above and depicted in FIG. 3-7 show SGCs with pre-amplifiers, amplifiers and label probes. It is understood that the same principles can be applied to an SGC where a pre-pre-amplifier component is included in the SGC, as disclosed herein (see, for example, FIGS. 5B, 5C, 6B and 6C for examples of SGCs with a pre-pre-amplifier layer).

In some embodiments, the SGCs that can be used in the disclosed methods comprise: (a) a set of pre-pre-amplifiers, wherein the pre-pre-amplifier set comprises one or more subsets of pre-pre-amplifiers, wherein the one or more pre-pre-amplifier subsets comprise a pre-pre-amplifier specific for each of the target probe pairs in the one or more target probe sets, wherein each pre-pre-amplifier comprises binding sites for the pair of target probes of one of the target probe sets and a plurality of binding sites for a pre-amplifier; (b) a set of pre-amplifiers, wherein the pre-amplifier set comprises one or more subsets of pre-amplifiers, wherein the one or more pre-amplifier subsets comprise a pre-amplifier specific for the pre-pre-amplifiers in the one or more pre-pre-amplifier subsets, wherein each pre-amplifier comprises binding sites for the pre-pre-amplifiers of one of the pre-pre-amplifier subsets and a plurality of binding sites for an amplifier; (d) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe; and (d) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets; wherein the one or more label probe subsets in each probe subset specific for a target nucleic acid comprise at least one label or a combination of labels that is different for each probe subset. This embodiment is similar to FIG. 3 except that a pre-pre-amplifier is included in the SGC.

In one embodiment, the set of target probes, pre-amplifiers, amplifiers and label probes each comprise two or more subsets. In another embodiment of such a method, the set of target probes, pre-amplifiers, amplifiers and label probes each comprise three or more subsets. In another embodiment, the set of target probes, pre-amplifiers, amplifiers and label probes each comprise four or more subsets.

In one embodiment, the target probe binding sites for the two or more subsets are intermingled on the target nucleic acid (see FIG. 5, top panel, except a pre-pre-amplifier is included in the SGC).

In some embodiments, the SGCs that can be used in the disclosed methods comprise: (a) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier; (b) a set of pre-amplifiers, wherein the pre-amplifier set comprises a plurality of pre-amplifiers, wherein the pre-amplifiers comprise binding sites for the pre-pre-amplifiers and a plurality of binding sites for an amplifier; (c) a set of amplifiers, wherein the amplifier set comprises a plurality of amplifiers, wherein the amplifiers comprise a binding site for the pre-amplifiers and a plurality of binding sites for a label probe or two or more distinct label probes; and d) a set of label probes, wherein the label probe set comprises a label probe or two or more distinct label probes, wherein each label probe comprises a label and a binding site for the amplifiers, wherein the labels in each distinct label probe are distinguishable between the distinct label probes; wherein the amplifier in each probe subset specific for a target nucleic acid comprises a binding site for a label or a combination of two or more distinct labels that is different for each probe subset. This embodiment is similar to FIG. 4A except that a pre-pre-amplifier is included in the SGC.

In one embodiment, the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier (similar to FIG. 7B except with a pre-pre-amplifier in the SGC).

In some embodiments, the SGCs that can be used in the disclosed methods comprise: (a) a set of pre-pre-amplifiers, wherein the pre-pre-amplifier set comprises a plurality of pre-pre-amplifiers, wherein each pre-pre-amplifier comprises binding sites for the pairs of target probes and a plurality of binding sites for a pre-amplifier; (b) a set of pre-amplifiers, wherein the pre-amplifier set comprises a plurality of pre-amplifiers, wherein the pre-amplifiers comprise binding sites for the pre-pre-amplifiers and a plurality of binding sites for amplifiers; (c) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe; and (d) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes; wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset. This embodiment is similar to FIG. 4B except that a pre-pre-amplifier is included in the SGC.

In one embodiment, the plurality of amplifiers comprise two or more distinct amplifiers, and wherein the binding sites on the pre-amplifier for the distinct amplifiers are intermingled (similar to FIG. 6B except with a pre-pre-amplifier layer in the SGC).

In some embodiments, the SGCs that can be used in the disclosed methods comprise: (a) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier or for two or more distinct pre-amplifiers; (b) a set of pre-amplifiers, wherein the pre-amplifier set comprises a plurality of pre-amplifiers, wherein the plurality of pre-amplifiers comprise a pre-amplifier comprising a binding site for the pre-pre-amplifiers and a plurality of binding sites for an amplifier, or wherein the plurality of pre-amplifiers comprise two or more distinct pre-amplifiers, wherein each distinct pre-amplifier comprises a binding site for the pre-pre-amplifiers and a plurality of binding sites for a distinct amplifier; (c) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for one of the distinct pre-amplifiers and a plurality of binding sites for a distinct label probe; and (d) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes; wherein the pre-pre-amplifier in each probe subset specific for a target nucleic acid comprises a plurality of binding sites for the pre-amplifier comprising a plurality of binding sites for the amplifier comprising a binding site for the label probe, or a plurality of binding sites for the two or more distinct pre-amplifiers each comprising a plurality of binding sites for one of the two or more distinct amplifiers comprising binding sites for one of the two or more distinct label probes, and wherein the label of the label probe or combination of two or more distinct labels of the two or more distinct label probes is different for each probe subset. This embodiment is similar to FIG. 4B, except that the combinatorial labeling is implemented at the level of one or more distinct pre-amplifiers binding to the pre-pre-amplifier, rather than at the level of one or more distinct amplifiers binding to the pre-amplifier, as shown in FIG. 4B.

In one embodiment, the plurality of pre-amplifiers comprise two or more distinct pre-amplifiers, and wherein the binding sites on the pre-pre-amplifier for the distinct pre-amplifiers are intermingled. This embodiment is similar to FIG. 6B except that the combinatorial labeling is implemented at the level of one or more distinct pre-amplifiers binding to the pre-pre-amplifier, rather than at the level of one or more distinct amplifiers binding to the pre-amplifier, as shown in FIG. 6B.

In some embodiments, the SGCs that can be used in the disclosed methods comprise: (a) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier; (b) a set of pre-amplifiers, wherein the pre-amplifier set comprises a plurality of pre-amplifiers, wherein the pre-amplifiers comprise binding sites for the pairs of target probes and a plurality of binding sites for an amplifier; (c) a set of amplifiers, wherein the amplifier set comprises a plurality of amplifiers, wherein the amplifiers comprise a binding site for the pre-amplifiers and a plurality of identical binding sites for a label probe; and (d) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes; wherein the amplifier in each probe subset specific for a target nucleic acid comprises a binding site for a label probe or a combination of two or more distinct label probes that is different for each probe subset. This embodiment is similar to FIG. 4C except that a pre-pre-amplifier is included in the SGC.

As disclosed herein, the components of the SGC are generally bound directly to each other. In the case of nucleic acid containing components, the binding reaction is generally by hybridization. In the case of a hybridization reaction, the binding between the components is direct. If desired, an intermediary component can be included such that the binding of one component to another is indirect, for example, the intermediary component contains complementary binding sites to bridge two other components.

In general, if a binding reaction is desired to be stable, the segments of complementary nucleic acid sequence between the components is generally in the range of 10 to 50 nucleotides, or greater, for example, 16 to 30 nucleotides, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, or greater. If a binding reaction is desired to be relatively unstable, such as when a collaborative hybridization binding reaction is employed, the segments of complementary nucleic acid sequence between the components is generally in the range of 5 to 18 nucleotides, for example, 5, 6, 7, 8, 9, 10, 11, 12, 13, 14, 15, 16, 17 or 18 nucleotides. The nucleotide lengths can be somewhat shorter or longer for a stable or unstable hybridization, depending on the sequence (for example, GC content) and the conditions employed in the assay. As disclosed herein, modified nucleotides such as Locked Nucleic Acid (LNA) or Bridged Nucleic Acid (BNA) can be used to increase the binding strength at the modified base, thereby allowing length of the binding segment to be reduced. Thus, with respect to the length of nucleic acid segments that are complementary to other nucleic acid segments, the lengths described herein can be reduced further, if desired. A person skilled in the art can readily determine appropriate probe designs, including length, the presence of modified nucleotides, and the like, to achieve a desired interaction between nucleic acid components.

In designing binding sites between two nucleic acid sequences comprising complementary sequences, the complementary sequences can optionally be designed to maximize the difference in melting temperature (dTm). This can be done by using melting temperature calculation algorithms known in the art (see, for example, SantaLucia, Proc. Natl. Acad. Sci. U.S.A. 95:1460-1465 (1998)). In addition, artificial modified bases such as Locked Nucleic Acid (LNA) or bridged nucleic acid (BNA) and naturally occurring 2′-O-methyl RNA are known to enhance the binding strength between complementary pairs (Petersen and Wengel, Trends Biotechnol. 21:74-81 (2003); Majlessi et al., Nucl. Acids Res. 26:2224-2229 (1998)). These modified bases can be strategically introduced into the binding site between components of an SGC, as desired.

One approach is to utilize modified nucleotides (LNA, BNA or 2′-O-methyl RNA). Because each modified base can increase the melting temperature, the length of binding regions between two nucleic acid sequences (i.e., complementary sequences) can be substantially shortened. The binding strength of a modified base to its complement is stronger, and the difference in melting temperatures (dTm) is increased. Yet another embodiment is to use three modified bases (for example, three LNA, BNA or 2′-O-methyl RNA bases, or a combination of two or three different modified bases) in the complementary sequences of a nucleic acid component or between two nucleic acid components, for example of a signal generating complex (SGC), that are to be hybridized. Such components can be, for example, a pre-pre-amplifier, a pre-amplifier, an amplifier, a label probe, or a pair of target probes.

The modified bases, such as LNA or BNA, can be used in the segments of selected components of SGC, in particular those mediating binding between nucleic acid components, which increases the binding strength of the base to its complementary base, allowing a reduction in the length of the complementary segments (see, for example, Petersen and Wengel, Trends Biotechnol. 21:74-81 (2003); U.S. Pat. No. 7,399,845). Artificial bases that expand the natural 4-letter alphabet such as the Artificially Expanded Genetic Information System (AEGIS; Yang et al., Nucl. Acids Res. 34 (21): 6095-6101 (2006)) can be incorporated into the binding sites among the interacting components of the SGC. These artificial bases can increase the specificity of the interacting components, which in turn can allow lower stringency hybridization reactions to yield a higher signal.

In the SGCs, each label probe (LP) comprises a segment that is detectable. As used herein, a “label” is a moiety that facilitates detection of a molecule. Common labels include fluorescent, luminescent, light-scattering, and/or colorimetric labels. Suitable labels include enzymes, and fluorescent and chromogenic moieties, as well as radionuclides, substrates, cofactors, inhibitors, chemiluminescent moieties, magnetic particles, rare earth metals, metal isotopes, and the like. In a particular embodiment, the label is an enzyme. Exemplary enzyme labels include, but are not limited to horseradish peroxidase (HRP), alkaline phosphatase (AP), β-galactosidase, glucose oxidase, and the like, as well as various proteases. Other labels include, but are not limited to, fluorophores, dinitrophenyl (DNP), and the like. Labels are known to those skilled in the art, as described, for example, in Hermanson, Bioconjugate Techniques, Academic Press, San Diego (1996), and U.S. Pat. Nos. 3,817,837; 3,850,752; 3,939,350; 3,996,345; 4,277,437; 4,275,149; and 4,366,241. Many labels are commercially available and can be used in methods and assays of the disclosure, including detectable enzyme/substrate combinations (Pierce, Rockford IL; Santa Cruz Biotechnology, Dallas TX; Life Technologies, Carlsbad CA). In a particular embodiment of the disclosure, the enzyme can utilize a chromogenic or fluorogenic substrate to produce a detectable signal, as described herein. Exemplary labels are described herein.

Any of a number of enzymes or non-enzyme labels can be utilized so long as the enzymatic activity or non-enzyme label, respectively, can be detected. The enzyme thereby produces a detectable signal, which can be utilized to detect a target nucleic acid. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have known chromogenic or fluorogenic substrates include various peptidases, where chromogenic or fluorogenic peptide substrates can be utilized to detect proteolytic cleavage reactions. The use of chromogenic and fluorogenic substrates is also known in bacterial diagnostics, including but not limited to the use of α- and β-galactosidase, β-glucuronidase, 6-phospho-β-D-galactoside 6-phosphogalactohydrolase, β-glucosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods provided herein.

Various chromogenic or fluorogenic substrates to produce detectable signals are known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-nitrophenyl phosphate (PNPP) for alkaline phosphatase; 1-methyl-3-indolyl-β-D-galactopyranoside and 2-methoxy-4-(2-nitrovinyl)phenyl β-D-galactopyranoside for β-galactosidase; 2-methoxy-4-(2-nitrovinyl)phenyl β-D-glucopyranoside for β-glucosidase; and the like. Exemplary fluorogenic substrates include, but are not limited to, 4-(trifluoromethyl)umbelliferyl phosphate for alkaline phosphatase; 4-methylumbelliferyl phosphate bis(2-amino-2-methyl-1,3-propanediol), 4-methylumbelliferyl phosphate bis (cyclohexylammonium) and 4-methylumbelliferyl phosphate for phosphatases; QuantaBlu™ and Quintolet for horseradish peroxidase; 4-methylumbelliferyl β-D-galactopyranoside, fluorescein di(β-D-galactopyranoside) and naphthofluorescein di-(β-D-galactopyranoside) for β-galactosidase; 3-acetylumbelliferyl β-D-glucopyranoside and 4-methylumbelliferyl-β-D-glucopyranoside for β-glucosidase; and 4-methylumbelliferyl-α-D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

Exemplary rare earth metals and metal isotopes suitable as a detectable label include, but are not limited to, lanthanide (III) isotopes such as ¹⁴¹Pr, ¹⁴²Nd, ¹⁴³Nd, ¹⁴⁴Nd, ¹⁴⁵Nd, ¹⁴⁶Nd, ¹⁴⁷Sm, ¹⁴⁸Nd, ¹⁴⁹Sm, ¹⁵⁰Nd, ¹⁵¹Eu, ¹⁵²Sm, ¹⁵³Eu, ¹⁵⁴Sm, ¹⁵⁵Gd, ¹⁵⁶Gd, ¹⁵⁸Gd, ¹⁵⁹Tb, ¹⁶⁰Gd, ¹⁶¹Dy, ¹⁶²Dy, ¹⁶³Dy, ¹⁶⁴Dy, ¹⁶⁵Ho, ¹⁶⁶Er, ¹⁶⁷Er, ¹⁶⁸Er, ¹⁶⁹Tm, ¹⁷⁰Er, ¹⁷¹Yb, ¹⁷²Yb, ¹⁷³Yb, ¹⁷⁴Yb, ¹⁷⁵Lu, and ¹⁷⁶Yb. Metal isotopes can be detected, for example, using time-of-flight mass spectrometry (TOF-MS) (for example, Fluidigm Helios and Hyperion systems, fluidigm.com/systems; South San Francisco, CA).

Biotin-avidin (or biotin-streptavidin) is a well-known signal amplification system based on the fact that the two molecules have extraordinarily high affinity to each other, and that one avidin/streptavidin molecule can bind four biotin molecules. Antibodies are widely used for signal amplification in immunohistochemistry and ISH. Tyramide signal amplification (TSA) is based on the deposition of a large number of haptenized tyramide molecules by peroxidase activity. Tyramine is a phenolic compound. In the presence of small amounts of hydrogen peroxide, immobilized horseradish peroxidase (HRP) converts the labeled substrate into a short-lived, extremely reactive intermediate. The activated substrate molecules then very rapidly react with and covalently bind to electron-rich moieties of proteins, such as tyrosine, at or near the site of the peroxidase binding site. In this way, many hapten molecules conjugated to tyramide can be introduced at the hybridization site in situ. Subsequently, the deposited tyramide-hapten molecules can be visualized directly or indirectly. Such a detection system is described in more detail, for example, in U.S. Pat. No. 8,658,361.

Embodiments described herein can utilize enzymes to generate a detectable signal using appropriate chromogenic or fluorogenic substrates. It is understood that, alternatively, a label probe can have a detectable label directly coupled to the nucleic acid portion of the label probe. Exemplary detectable labels are known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; naphthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA); Alexa Fluor dyes (Molecular Probes), and the like; 4,4-difluoro-4-bora-3a,4a-diaza-s-indacene (BODIPY™) and derivatives thereof (Molecular Probes); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences); ATTO dyes (ATTO-TEC GmbH); and the like. Particular dyes include ATTO 390, DyLight 395XL, Alexa Fluor 405, ATTO 425, Alexa Fluor 430, DyLight430, ATTO 465, ATTO 488, Alexa Fluor 488, ATTO 490LS, ATTO 495, ATTO 514, ATTO 520, ATTO 532, Alexa Fluor 532, ATTO Rho6G, ATTO 542, Alexa Fluor 546, ATTO 550, DyLight 550, Alexa Fluor 555, ATTO 565, Alexa Fluor 568, ATTO Rho3B, ATTO Rho11, ATTO Rho12, ATTO Thio12, ATTO Rho101, ATTO 590, ATTO 594, Alexa Fluor 594, Dylight 594, ATTO Rho13, ATTO 610, Alexa Fluor 610, ATTO 620, ATTO Rho14, ATTO 633, Alexa Fluor 633, Alexa Fluor 635, ATTO 643, ATTO 647, Alexa Fluor 647, ATTO 647N, DyLight650, ATTO 655, ATTO Oxa12, Alexa Fluor 660, ATTO 665, ATTO 680, Alexa Fluor 680, ATTO 700, DyLight700, AlexaFluor700, ATTO 725, ATTO 740, Alexa Fluor 750, DyLight 755, Cyan 500 NHS-Ester, and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

As disclosed herein, the methods provided herein can be used for concurrent detection of multiple target nucleic acids. In the case of using fluorophores as labels, the fluorophores to be used for detection of multiple target nucleic acids are selected so that, within each round of labeling and detection, each of the fluorophores is distinguishable and can be detected concurrently. Such fluorophores are selected to have spectral separation of the emissions so that distinct labeling of the target nucleic acids can be detected concurrently. Methods of selecting suitable distinguishable fluorophores for use in methods of the disclosure are known in the art (see, for example, Johnson and Spence, “Molecular Probes Handbook, a Guide to Fluorescent Probes and Labeling Technologies,” 11th ed., Life Technologies (2010)). In some embodiments, 1, 2, 3, 4, 5, 6, 7, 8, 9, or 10 distinguishable fluorophores can be used. For example, selection of one to six fluorophores with excitation maxima at or near 488 nm, 550 nm, 594 nm, 650 nm, 700 nm, and 750 nm may be suitable in combination. Particular examples of fluorophores with excitation maxima at or near 488 nm include, e.g., Alexa Fluor 488, DyLight 488, ATTO 488, and Cy2. Particular examples of fluorophores with excitation maxima at or near 550 nm include, e.g., DyLight 550, Alexa Fluor 546, ATTO 550, Cy3, and rhodamine. Particular examples of fluorophores with excitation maxima at or near 594 nm include, e.g., Dylight 594, Alexa Fluor 594, ATTO 594, and Texas Red. Particular examples of fluorophores with excitation maxima at or near 650 nm include, e.g., DyLight 650, ATTO 647, Alexa Fluor 647, ATTO 647N, and ATTO 655. Particular examples of fluorophores with excitation maxima at or near 700 nm include, e.g., ATTO 700, DyLight700, and AlexaFluor700. Particular examples of fluorophores with excitation maxima at or near 750 nm include, e.g., Alexa Fluor 750, Cy7, and DyLight 755, and ATTO 740.

Methods such as microscopy, cytometry (e.g., mass cytometry, cytometry by time of flight (CyTOF), flow cytometry), or spectroscopy can be used to visualize chromogenic, fluorescent, or metal detectable signals associated with the respective target nucleic acids. In general, either chromogenic substrates or fluorogenic substrates, or chromogenic or fluorescent labels, or rare earth metal isotopes, will be utilized for a particular assay, if different labels are used in the same assay, so that a single type of instrument can be used for detection of nucleic acid targets in the same sample.

As disclosed herein, the label can be designed such that the labels are optionally cleavable. As used herein, a “cleavable label” refers to a label that is attached or conjugated to a label probe so that the label can be removed, for example, in order to use the same label in a subsequent round of labeling and detecting of target nucleic acids. Generally, the cleavable labels are conjugated to the label probe by a chemical linker that is cleavable. Methods of conjugating a label to a label probe so that the label is cleavable are known to those skilled in the art (see, for example, Hermanson, Bioconjugate Techniques, Academic Press, San Diego (1996); Daniel et al., BioTechniques 24(3):484-489 (1998)). One particular system of labeling oligonucleotides is the FastTag™ system (Daniel et al., supra, 1998; Vector Laboratories, Burlinghame CA). Various cleavable moieties can be included in the linker so that the label can be cleaved from the label probe. Such cleavable moieties include groups that can be chemically, photochemically or enzymatically cleaved. Cleavable chemical linkers can include a cleavable chemical moiety, such as disulfides, which can be cleaved by reduction, glycols or diols, which can be cleaved by periodate, diazo bonds, which can be cleaved by dithionite, esters, which can be cleaved by hydroxylamine, sulfones, which can be cleaved by base, and the like (see Hermanson, supra, 1996). One particularly useful cleavable linker is a linker containing a disulfide bond, which can be cleaved by reducing the disulfide bond. In other embodiments, the linker can include a site for cleavage by an enzyme. For example, the linker can contain a proteolytic cleavage site. Generally, such a cleavage site is for a sequence-specific protease. Such proteases include, but are not limited to, human rhinovirus 3C protease (cleavage site LEVLFQ/GP), enterokinase (cleavage site DDDDK/), factor Xₐ (cleavage site IEGR/), tobacco etch virus protease (cleavage site ENLYFQ/G), and thrombin (cleavage site LVPR/GS) (see, for example, Oxford Genetics, Oxford, UK). Another cleavable moiety can be, for example, uracil-DNA (DNA containing uracil), which can be cleaved by uracil-DNA glycosylase (UNG) (see, for example, Sidorenko et al., FEBS Lett. 582(3):410-404 (2008)).

The cleavable labels can be removed by applying an agent, such as a chemical agent or light, to cleave the label and release it from the label probe. As discussed above, useful cleaving agents for chemical cleavage include, but are not limited to, reducing agents, periodate, dithionite, hydroxylamine, base, and the like (see Hermanson, supra, 1996). One useful method for cleaving a linker containing a disulfide bond is the use of tris(2-carboxyethyl)phosphine (TCEP) (see Moffitt et al., Proc. Natl. Acad. Sci. USA 113:11046-11051 (2016)). In one embodiment, TCEP is used as an agent to cleave a label from a label probe.

c. Removal of Signals

Methods disclosed herein include a step of removing signals generated by the detectable labels from the sample. In some embodiments, the step of removing signals generated by the detectable labels comprises removing the labels from the label probes, e.g., by cleaving cleavable labels as discussed above. In other embodiments, the step of removing the signals generated by the detectable labels comprises removing the SGCs from the sample, e.g., by disrupting binding of the SGCs bound to the target nucleic acid. In some embodiments, the SGCs are removed from the sample by contacting the sample with an acid reagent that disrupts hybridization between the SGCs and the target nucleic acid.

The acid reagent comprises an acid. Exemplary acids suitable for use in an acid reagent include, but are not limited to, formic acid, acetic acid, propionic acid, butyric acid, valeric acid, oxalic acid, malonic acid, succinic acid, malic acid, tartaric acid, citric acid, and the like. The acid reagent generally includes an acid at a concentration of about 5-40% acid (vol/vol). In some embodiments, the acid reagent comprises an acid at a concentration of about 20-30% (vol/vol). For example, in some embodiments, the acid reagent comprises an acid at a concentration of about 5%, about 6%, about 7%, about 8%, about 9%, about 10%, about 11%, about 12%, about 13%, about 14%, about 15%, about 16%, about 17%, about 18%, about 19%, about 20%, about 21%, about 22,%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% acid (vol/vol). In a particular embodiment, the acid reagent comprises acetic acid at a concentration of about 5-40%, or about 20-30%, e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22,%, about 23%, about 24%, about 25%, about 26%, about 27%, about 28%, about 29%, about 30%, about 31%, about 32%, about 33%, about 34%, about 35%, about 36%, about 37%, about 38%, about 39% or 40% acetic acid (% vol/vol), or any concentration therebetween.

In some embodiments, the acid reagent further comprises one or more components selected from salts, chelating agents, buffers, and any combination thereof. In one embodiment, the acid reagent further comprises saline sodium citrate (SSC), where 20×SSC corresponds to 3.0 M NaCl and 0.3 M sodium citrate, at pH 7.0 (see Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999)). In one embodiment, the acid reagent further comprises 1×SSC to 13×SSC. For example, in some embodiments, the acid reagent further comprises SSC at 1×, 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, 4.9×, 5×, 5.1×, 5.2×, 5.3×, 5.4×, 5.5×, 5.6×, 5.7×, 5.8×, 5.9×, 6×, 6.1×, 6.2×, 6.3×, 6.4×, 6.5×, 6.6×, 6.7×, 6.8×, 6.9×, 7×, 7.1×, 7.2×, 7.3×, 7.4×, 7.5×, 7.6×, 7.7×, 7.8×, 7.9×, 8×, 8.1×, 8.2×, 8.3×, 8.4×, 8.5×, 8.6×, 8.7×, 8.8×, 8.9×, 9×, 9.1×, 9.2×, 9.3×, 9.4×, 9.5×, 9.6×, 9.7×, 9.8×, 9.9×, 10×, 10.1×, 10.2×, 10.3×, 10.4×, 10.5×, 10.6×, 10.7×, 10.8×, 10.9×, 11×, 11.1×, 11.2×, 11.3×, 11.4×, 11.5×, 11.6×, 11.7×, 11.8×, 11.9×, 12×, 12.1×, 12.2×, 12.3×, 12.4×, 12.5×, 12.6×, 12.7×, 12.8×, 12.9×, or 13×, or any concentration therebetween.

In some embodiments, the acid reagent further comprises saline sodium phosphate EDTA (SSPE), which is a mixture of sodium chloride, sodium phosphate, and ethylenediaminetetraacetic acid (EDTA), where 20×SSPE corresponds to 3.0 M sodium chloride, 0.2 M sodium hydrogen phosphate (NaH₂PO₄), and 0.02 M ethylenediaminetetraacetic acid (EDTA), pH 7.4 (see Sambrook et al., Molecular Cloning: A Laboratory Manual, Third Ed., Cold Spring Harbor Laboratory, New York (2001)). In some embodiments, the acid reagent further comprises 1×SSPE to 13×SSPE. For example, the acid reagent comprises SSPE at 1×, 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, 4.9×, 5×, 5.1×, 5.2×, 5.3×, 5.4×, 5.5×, 5.6×, 5.7×, 5.8×, 5.9×, 6×, 6.1×, 6.2×, 6.3×, 6.4×, 6.5×, 6.6×, 6.7×, 6.8×, 6.9×, 7×, 7.1×, 7.2×, 7.3×, 7.4×, 7.5×, 7.6×, 7.7×, 7.8×, 7.9×, 8×, 8.1×, 8.2×, 8.3×, 8.4×, 8.5×, 8.6×, 8.7×, 8.8×, 8.9×, 9×, 9.1×, 9.2×, 9.3×, 9.4×, 9.5×, 9.6×, 9.7×, 9.8×, 9.9×, 10×, 10.1×, 10.2×, 10.3×, 10.4×, 10.5×, 10.6×, 10.7×, 10.8×, 10.9×, 11×, 11.1×, 11.2×, 11.3×, 11.4×, 11.5×, 11.6×, 11.7×, 11.8×, 11.9×, 12×, 12.1×, 12.2×, 12.3×, 12.4×, 12.5×, 12.6×, 12.7×, 12.8×, 12.9×, or 13×, or any concentration therebetween.

In some embodiments, the acid reagent further comprises a phosphate buffer, e.g., 10-500 mM sodium phosphate, with a pH in the range of about 7 to about 8 (e.g., a pH of about 7.8). For example, in some embodiments, the acid reagent further comprises sodium phosphate at about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM, or any concentration therebetween.

In another embodiment, the acid reagent further comprises sodium chloride (NaCl), e.g., at a concentration of about 10 mM to about 6 M. For example, in some embodiments, the acid reagent comprises sodium chloride at a concentration of about 10 mM, about 50 mM, about 100 mM, about 150 mM, about 200 mM, about 250 mM, about 300 mM, about 350 mM, about 400 mM, about 450 mM, about 500 mM, about 550 mM, about 600 mM, about 650 mM, about 700 mM, about 750 mM, about 800 mM, about 850 mM, about 900 mM, about 950 mM, about 1 M, about 1.1 M, about 1.2 M, about 1.3 M 1.4 M, about 1.5 M, about 1.6 M, about 1.7 M, about 1.8 M, about 1.9 M, about 2 M, about 2.1 M, about 2.2 M, about 2.3 M, about 2.4 M, about 2.5 M, about 2.6 M, about 2.7 M, about 2.8 M, about 2.9 M, about 3 M, about 3.1 M, about 3.2 M, about 3.3 M, about 3.4 M, about 3.5 M, about 3.6 M, about 3.7 M, about 3.8 M, about 3.9 M, about 4 M, about 4.1 M, about 4.2 M, about 4.3 M, about 4.4 M, about 4.5 M, about 4.6 M, about 4.7 M, about 4.8 M, about 4.9 M, about 5 M, about 5.1 M, about 5.2 M, about 5.3 M, about 5.4 M, about 5.5 M, about 5.6 M, about 5.7 M, about 5.8 M, about 5.9 M, or about 6 M, and the like, or any concentration therebetween.

In some embodiments, the acid reagent comprises 5-40% acid and 1×-12.8×SSC, wherein the acid and the SSC can each be present independently at any concentration disclosed herein. For example, in some embodiments, the acid reagent comprises 20-30% acid and 3.2×-12.8×SSC. In some embodiments, the acid reagent comprises 20% acid and 3.2×SSC. In some embodiments, the acid reagent comprises 20% acid and 6.4×SSC. In some embodiments, the acid reagent comprises 20% acid and 12.8×SSC. In some embodiments, the acid reagent comprises 30% acid and 3.2×SSC. In some embodiments, the acid reagent comprises 30% acid and 6.4×SSC. In some embodiments, the acid reagent comprises 30% acid and 12.8×SSC.

In some embodiments, the acid reagent comprises 5-40% acetic acid and 1×-12.8×SSC, wherein the acetic acid and the SSC can each be present independently at any concentration disclosed herein. For example, in some embodiments, the acid reagent comprises 20% acetic acid and 3.2×SSC. In some embodiments, the acid reagent comprises 20% acetic acid and 6.4×SSC. In some embodiments, the acid reagent comprises 20% acetic acid and 12.8×SSC. In some embodiments, the acid reagent comprises 30% acetic acid and 3.2×SSC. In some embodiments, the acid reagent comprises 30% acetic acid and 6.4×SSC. In some embodiments, the acid reagent comprises 30% acetic acid and 12.8×SSC.

In some embodiments, the step of removing SGCs from a sample by adding an acid reagent to effect disruption of hybridization between the SGCs and the target nucleic acids in the sample is carried out at room temperature. In other embodiments, this step can be conducted at a temperature just below or above room temperature. Thus, the acid reagent can be applied to a sample, for example, at temperature of about 4 °C to about 40 °C. For example, the methods can be carried out at a temperature of about 4 °C, about 5 °C, about 6 °C, about 7 °C, about 8 °C, about 9 °C, about 10 °C, about 11 °C, about 12 °C, about 13 °C, about 14 °C, about 15 °C, about 16 °C, about 17 °C, about 18 °C, about 19 °C, about 20 °C, about 21 °C, about 22 °C, about 23 °C, about 24 °C, about 25 °C, about 26 °C, about 27 °C, about 28 °C, about 29 °C, about 30 °C, about 31 °C, about 32 °C, about 33 °C, about 34 °C, about 35 °C, about 36 °C, about 37 °C, about 38 °C, about 39 °C, about 40 °C, or any range therebetween.

In some embodiments, the step of removing SGCs from a sample by adding an acid reagent to effect disruption of hybridization between the SGCs and the target nucleic acids in the sample is carried out for a period of time of about 1 minute to about 30 minutes, or about 3 minutes to about 10 minutes, for example, about 1 minute, about 2 minute, about 3 minute, about 4 minute, about 5 minute, about 6 minute, about 7 minute, about 8 minute, about 9 minute, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, or any range therebetween. In some embodiments, the acid reagent treatment is repeated 1 to 10 times.

In some embodiments, the step of removing SGCs from a sample by adding an acid reagent to effect disruption of hybridization between the SGCs and the target nucleic acids in the sample is carried out 1, 2, 3, 4, 5, 6, 7, 8, 9, 10 or 11 times (i.e., repeated up to 10 times). In some embodiments, this step is carried out 1, 2, 3, 4, 5 or 6 times (i.e., repeated up to 5 times). In some embodiments, this step is carried out 1, 2, or 3 times (i.e., repeated up to 2 times). In some embodiments, the acid reagent is contacted with the sample sequentially without removing the acid reagent (for example, by aspirating the acid reagent from the sample) or washing the sample (for example, washing the sample between applications of the acid reagent). In other embodiments, the acid reagent can be removed from contact with the sample, for example, by aspirating the acid reagent away from the sample or washing the sample with a suitable buffer. Suitable wash buffers include, but are not limited to, a buffer used routinely in in situ hybridization assays. It is understood that the conditions for removal of probes bound to target nucleic acids in a sample can be readily determined depending on the components and concentration of components of the acid reagent, the time of incubation of the acid reagent with the sample, and the number of times the incubation is repeated, as disclosed herein. The effectiveness of the removal of the probes from a sample can be readily determined by analyzing the sample using the same method used to detect a target nucleic acid to see if residual probe can be detected (see Examples I and II). If residual probe is still present, the acid reagent treatment merely needs to be repeated until the previously detected probes are no longer detected or are detected at a sufficiently low level to permit detectable labeling of a target nucleic acid with the same label in a subsequent round of labeling. Similarly, the number of times that a sample can be treated with the acid reagent while preserving cell morphology and integrity of the nucleic acids in the sample to permit subsequent detection of nucleic acids can be readily determined by performing repeated acid reagent treatments of a sample for a given acid reagent and under a given set of conditions and determining whether or not target nucleic acids can still be detected, for example, by determining the ability to detect a positive control nucleic acid in the sample or determining that a similar cell morphology can be detected in the sample after the sample has been treated one or more times with the acid reagent (see Examples I and II). Once a set of conditions of incubation time and number of repeats of applying a given acid reagent has been determined, the conditions can be applied to other samples. As disclosed herein, a range of acid reagents and conditions were tested and shown to be effective at disrupting hybridization between probes and target nucleic acids that preserved cell morphology and nucleic acid integrity such that a new round of detection of target nucleic acids could be applied.

d. Targets and Samples

In an embodiment of the disclosure, the target nucleic acids detected by the methods of the invention can be any nucleic acid present in the cell sample, including but not limited to, RNA, including messenger RNA (mRNA), micro RNA (miRNA), ribosomal RNA (rRNA), mitochondrial RNA, non-coding RNA, and the like, or DNA, and the like. In a particular embodiment, the nucleic acid is RNA. In the disclosed methods for multiplex detection of nucleic acids, it is understood the target nucleic acids can independently be DNA or RNA. In other words, the target nucleic acids to be detected can be, but are not necessarily, the same type of nucleic acid. Thus, the target nucleic acids to be detected in an assay of the invention can be DNA and RNA. In the case where the target nucleic acids are RNA, the target nucleic acids can independently be selected from the group consisting of messenger RNA (mRNA), micro RNA (miRNA), ribosomal RNA (rRNA), mitochondrial RNA, and non-coding RNA. Thus, the target nucleic acids can independently be DNA or any type of RNA.

As described herein, the methods of the disclosure generally relate to in situ detection of target nucleic acids. General embodiments of such methods are well-known to those skilled in the art (see, for example, US 2008/0038725; US 2009/0081688; Hicks et al., J. Mol. Histol. 35:595-601 (2004)). As used herein, “in situ hybridization” or “ISH” refers to a type of hybridization that uses a directly or indirectly labeled complementary DNA or RNA strand, such as a probe, to bind to and localize a specific nucleic acid, such as DNA or RNA, in a sample, in particular a portion or section of tissue or cells (in situ). The probe types can be double stranded DNA (dsDNA), single stranded DNA (ssDNA), single stranded complimentary RNA (sscRNA), messenger RNA (mRNA), micro RNA (miRNA), ribosomal RNA, mitochondrial RNA, and/or synthetic oligonucleotides. The term “fluorescent in situ hybridization” or “FISH” refers to a type of ISH utilizing a fluorescent label. The term “chromogenic in situ hybridization” or “CISH” refers to a type of ISH with a chromogenic label. ISH, FISH and CISH methods are well known to those skilled in the art (see, for example, Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000)).

In the disclosed methods for detection of a plurality of targets in a sample, the sample comprises cells that are optionally fixed and/or permeabilized before hybridization of the target probes. Fixing and permeabilizing cells can facilitate retaining the nucleic acid targets in the cell and permit the target probes, label probes, amplifiers, pre-amplifiers, pre-pre-amplifiers, and so forth, to enter the cell and reach the target nucleic acid molecule. The cell is optionally washed to remove materials not captured to a nucleic acid target. The cell can be washed after any of various steps, for example, after hybridization of the target probes to the nucleic acid targets to remove unbound target probes, after hybridization of the pre-pre-amplifiers, pre-amplifiers, amplifiers, and/or label probes to the target probes, and the like. Methods for fixing and permeabilizing cells for in situ detection of nucleic acids, as well as methods for hybridizing, washing and detecting target nucleic acids, are also well known in the art (see, for example, US 2008/0038725; US 2009/0081688; Hicks et al., J. Mol. Histol. 35:595-601 (2004); Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000); Shapiro, Practical Flow Cytometry 3rd ed., Wiley-Liss, New York (1995); Ormerod, Flow Cytometry, 2nd ed., Springer (1999)). Exemplary fixing agents include, but are not limited to, aldehydes (formaldehyde, glutaraldehyde, and the like), acetone, alcohols (methanol, ethanol, and the like). Exemplary permeabilizing agents include, but are not limited to, alcohols (methanol, ethanol, and the like), acids (glacial acetic acid, and the like), detergents (Triton, NP-40, Tween™ 20, and the like), saponin, digitonin, Leucoperm™ (BioRad, Hercules, CA), and enzymes (for example, lysozyme, lipases, proteases and peptidases). Permeabilization can also occur by mechanical disruption, such as in tissue slices.

For in situ detection of double stranded nucleic acids, generally the sample is treated to denature the double stranded nucleic acids in the sample to provide accessibility for the target probes to bind by hybridization to a strand of the target double stranded nucleic acid. Conditions for denaturing double stranded nucleic acids are well known in the art, and include heat and chemical denaturation, for example, with base (NaOH), formamide, dimethyl sulfoxide, and the like (see Wang et al., Environ. Health Toxicol. 29:e2014007 (doi: 10.5620/eht.2014.29.e2014007) 2014; Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999)). For example, NaOH, LiOH or KOH, or other high pH buffers (pH>11) can be used to denature double stranded nucleic acids such as DNA. In addition, heat and chemical denaturation methods can be used in combination.

Such in situ detection methods can be used on tissue specimens immobilized on a glass slide, on single cells in suspension such as peripheral blood mononucleated cells (PBMCs) isolated from blood samples, and the like. Tissue specimens include, for example, tissue biopsy samples. Blood samples include, for example, blood samples taken for diagnostic purposes. In the case of a blood sample, the blood can be directly analyzed, such as in a blood smear, or the blood can be processed, for example, lysis of red blood cells, isolation of PBMCs or leukocytes, isolation of target cells, and the like, such that the cells in the sample analyzed by the disclosed methods are in a blood sample or are derived from a blood sample. Similarly, a tissue specimen can be processed, for example, the tissue specimen minced and treated physically or enzymatically to disrupt the tissue into individual cells or cell clusters. Additionally, a cytological sample can be processed to isolate cells or disrupt cell clusters, if desired. Thus, the tissue, blood and cytological samples can be obtained and processed using methods well known in the art. The disclosed methods can be used in diagnostic applications to identify the presence or absence of pathological cells based on the presence or absence of a nucleic acid target that is a biomarker indicative of a pathology.

Any of a number of suitable samples can be used for detecting target nucleic acids using the disclosed methods. In some embodiments, the sample is a biological sample or tissue sample. Such a sample can be obtained from a biological subject, including a sample of biological tissue or fluid origin that is collected from an individual or some other source of biological material such as biopsy, autopsy or forensic materials. A biological sample also includes samples from a region of a biological subject containing or suspected of containing precancerous or cancer cells or tissues, for example, a tissue biopsy, including fine needle aspirates, blood sample or cytological specimen. Such samples can be, but are not limited to, organs, tissues, tissue fractions and/or cells isolated from an organism such as a mammal. Exemplary biological samples include, but are not limited to, a cell culture, including a primary cell culture, a cell line, a tissue, an organ, an organelle, a biological fluid, and the like. Additional biological samples include but are not limited to a skin sample, tissue biopsies, including fine needle aspirates, cytological samples, stool, bodily fluids, including blood and/or serum samples, saliva, semen, and the like. Such samples can be used for medical or veterinary diagnostic purposes. A sample can also be obtained from other sources, for example, food, soil, surfaces of objects, and the like, and other materials for which detection of target nucleic acids is desired. Thus, the disclosed methods can be used for detection of one or more pathogens, such as a virus, a bacterium, a fungus, a single celled organism such as a parasite, and the like, from a biological sample obtained from an individual or other sources.

Collection of cytological samples for analysis are well known in the art (see, for example, Dey, “Cytology Sample Procurement, Fixation and Processing” in Basic and Advanced Laboratory Techniques in Histopathology and Cytology pp. 121-132, Springer, Singapore (2018); “Non-Gynecological Cytology Practice Guideline” American Society of Cytopathology, Adopted by the ASC executive board Mar. 2, 2004). Methods for processing samples for analysis of cervical tissue, including tissue biopsy and cytology samples, are well known in the art (see, for example, Cecil Textbook of Medicine, Bennett and Plum, eds., 20th ed., WB Saunders, Philadelphia (1996); Colposcopy and Treatment of Cervical Intraepithelial Neoplasia: A Beginner's Manual, Sellors and Sankaranarayanan, eds., International Agency for Research on Cancer, Lyon, France (2003); Kalaf and Cooper, J. Clin. Pathol. 60:449-455 (2007); Brown and Trimble, Best Pract. Res. Clin. Obstet. Gynaecol. 26:233-242 (2012); Waxman et al., Obstet. Gynecol. 120:1465-1471(2012); Cervical Cytology Practice Guidelines TOC, Approved by the American Society of Cytopathology (ASC) Executive Board, Nov. 10, 2000)). In one embodiment, the cytological sample is a cervical sample, for example, a pap smear. In one embodiment, the sample is a fine needle aspirate.

In particular embodiments, the sample is a tissue specimen or is derived from a tissue specimen. In some embodiments, the tissue specimen is a formalin-fixed paraffin-embedded (FFPE) sample. In some embodiments, the tissue specimen is fresh frozen. In some embodiments, the tissue specimen is prepared with a fixative. In some embodiments, the tissue specimen is prepared with a crosslinking fixative. In other particular embodiments of the invention, the sample is a blood sample or is derived from a blood sample. In still other particular embodiments of the invention, the sample is a cytological sample or is derived from a cytological sample.

In some embodiments, the method for detecting a target nucleic acid in a cell provided herein comprises a pretreatment step before hybridization of the target probe sets. In some embodiments, the pretreatment step comprises a blocking step where certain blocking agent(s) is/are applied to block certain endogenous components of the cell thus reducing assay background. For example, hydrogen peroxide is a blocking agent when horseradish peroxidase (HRP) is used as detection enzyme in the later steps. Hydrogen peroxide is added to inactivate the endogenous HRP activity in the sample, thus reducing assay background. In a specific embodiment, this blocking step is added as the first step in the pretreatment right after deparaffinization. In some embodiments, the pretreatment step comprises an epitope retrieval step, where certain epitope retrieval buffer(s) can be added to unmask the target nucleic acid. In some embodiments, the epitope retrieval step comprises heating the sample. In some embodiments, the epitope retrieval step comprises heating the sample to 50 °C to 100 °C. In one embodiment, the epitope retrieval step comprises heating the sample to about 88 °C. In some embodiments, the pretreatment step comprises a permeabilization step to retain the nucleic acid targets in the cell and to permit the target probe(s), signal-generating complex, etc. to enter the cell. In some embodiments, the permeabilization step comprises a digestion with a protease. Detergents (e.g., Triton X-100 or SDS) and Proteinase K can also be used to increase the permeability of the fixed cells. Detergent treatment, usually with Triton X-100 or SDS, is frequently used to permeate the membranes by extracting the lipids. Proteinase K is a nonspecific protease that is active over a wide pH range and is not easily inactivated. It is used to digest proteins that surround the target mRNA. Optimal concentrations and durations of treatment can be experimentally determined as is known in the art. A cell washing step can follow, to remove the dissolved materials produced in the any steps in the pretreatment step. In some embodiments, the sample is in a formalin-fixed paraffin embedded tissue, a deparaffinization step is needed, when paraffin is removed.

e. Codetection Methods

The methods described herein generally relates to detection of multiple target nucleic acids in a sample. It is understood that the disclosed can additionally be applied to detecting multiple target nucleic acids and optionally other molecules in the sample, in particular in the same cell as the target nucleic acid. For example, in addition to detecting multiple target nucleic acids, proteins expressed in a cell can also concurrently be detected using a similar rationale as described herein for detecting target nucleic acids. In this case, in one or more rounds of detection of multiple target nucleic acids, and optionally one or more proteins expressed in a cell can be detected, for example, by using a detectable label to detect the protein. For example, if the protein is being detected in an earlier round of target nucleic acid detection, the protein can be detected with a cleavable label, similar to that used for detecting target nucleic acids. If the protein is being detected in the last round of detection, the label does not need to be cleavable. Detection of proteins in a cell are well known to those skilled in the art, for example, by detecting the binding of protein-specific antibodies using any of the well-known detection systems, including those described herein for detection of target nucleic acids. Detection of target nucleic acids and protein in the same cell has been described (see also Schulz et al., Cell Syst. 6(1):25-36 (2018)).

In some embodiments, proteins can be co-detected using antibodies (or fragments thereof) that are conjugated to an oligonucleotide, using the same types of SGCs that are disclosed herein. Such methods are disclosed in U.S. Provisional Patent Application No. 63/301,711, which is incorporated herein by reference.

### Kits

Embodiments of the present disclosure also include a kit for carrying out the methods disclosed herein. The kit can include the components described herein for performing a multiplexed in situ hybridization reaction, e.g., sets of target probes, SGCs (including label probes and amplifiers, pre-amplifiers, and/or pre-pre-amplifiers). Such components have been discussed extensively herein. The kit further comprises at least one compound or composition for removing signals generated by the detectable labels from the sample, to facilitate additional rounds of labeling and detection as discussed herein.

For example, in some embodiments, the kit further comprises a reagent for cleaving labels from a label probe, such as a reducing agent, periodate, dithionite, hydroxylamine, base, or the like. In one embodiment, the cleaving reagent is tris(2-carboxyethyl)phosphine (TCEP).

In some embodiments, the kit further comprises a reagent that disrupts hybridization between SGCs and a target nucleic acid, such as an acid reagent. Exemplary acids suitable for use in an acid reagent include, but are not limited to, formic acid, acetic acid, propionic acid, butyric acid, valeric acid, oxalic acid, malonic acid, succinic acid, malic acid, tartaric acid, citric acid, and the like. The acid reagent generally includes an acid at a concentration of about 5-40% acid (vol/vol). Further information regarding acid reagents is provided above; any such reagents can be included in the kits.

In some embodiments, the kit further comprises other agents or materials for performing RNA ISH, including fixing agents and agents for treating samples for preparing hybridization, agents for washing samples, and so on.

The kit may further comprise packaging material, which refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits provided herein can include labels or inserts. Labels or inserts include information on a condition, disorder, disease, or symptom for which the kit component may be used for. Labels or inserts can include instructions for a clinician or for a subject to use one or more of the kit components in a method, treatment protocol, or therapeutic regimen. In some embodiments, the kit can be used for identification of tissues and cell types. In some embodiments, the kit can be used for identification of different stages of development. In some embodiments, the kit can be used for detection of clinical biomarkers for cancers. In some embodiments, the kit can be used for diagnosing a disease or disorder based on the expression of one or more altered small RNAs or the presence of pathogen-associated small RNAs. In some embodiments, the kit can be used for characterization of adult tissue. In some embodiments, the kit can be used for detection of clinical biomarkers for pathogen diagnosis. In some embodiments, the kit can be used for detection and characterization of small RNA-based therapies. In some embodiments, the kit can be used for confirmation of the initial efficiency of small RNA-based therapies. In some embodiments, the kit can be used to continue monitoring the efficiency of small RNA-based therapies. In some embodiments, the kit can be used for determining the efficiency of small RNA-based therapies. In some embodiments, the kit can be used for detecting the presence, localizing, and quantifying siRNAs. In some embodiments, the kit can be used for detecting the presence, localizing, and quantifying ASO molecules. In some embodiments, the kit can be used for detection and identification of pathogen-derived small RNAs.

### Image Processing

Embodiments of the present disclosure also include a method for enhancing detection of a target (e.g., a target nucleic acid). In some embodiments, the method includes an image processing method, such as the methods described in International Patent Application PCT/US22/24975, which is herein incorporated by reference. The method is implemented at least in part with a computer having corresponding instructions stored on a memory (i.e., a non-transitory computer readable medium). The final images, and in some embodiments the intermediate images, from the method are stored in a memory. In some embodiments, the memory is accessible by a network. In some embodiments, user input or instructions are receivable or accessible over the network.

The method includes imaging a sample with a target signal to create a probe image and imaging a sample with no target signal to create a background image (i.e., “blank image”). In some embodiments, a “blank image” is an image that includes RNA blocking molecules of the present disclosure. In some embodiments, a “blank image” is an image that does not include RNA blocking molecules of the present disclosure. In some embodiments, the imaging utilizes a fluorescent microscope coupled to a computer via a network. In some embodiments, the target signal is obtained by subjecting the sample to a fluorescent in situ hybridization assay and/or an immunofluorescence assay. In some embodiments, the background image with no target signal is obtained by removing the target signal from the sample (i.e., by a cleaving process). In other embodiments, the background image with no target signal is obtained before the assay is performed. In some embodiments, the target signal comprises a fluorescent label bound to a target nucleic acid. In other embodiments, the target signal comprises a fluorescent label bound to a target peptide or polypeptide.

The method can also include registering the probe image and the background image (e.g., with or without RNA blocking molecules). Potential background fluorescence discrepancy between the probe image and the background image creates spatial pattern mismatches that occur due to whole sample movement between different rounds of image acquisition. To remove such discrepancies, image registration techniques (e.g., phase correlation) are utilized. Robust image registration utilizes detection and matching of image features to compensate for any global sample movement (i.e., translation and rotation).

The method further includes modifying the background image (e.g., with or without RNA blocking molecules) to create an adjusted background image (e.g., transformed, intensity-adjusted blank image) based on at least one image metric. As explained further herein, the at least one image metric is a ratio factor, a multiplication factor, a local maximum value transform, and any other suitable metric. In some embodiments, the method includes a single image metric. In other embodiments, the method includes a combination of image metrics.

In some embodiments, the method further includes subtracting the adjusted background image (e.g., with or without RNA blocking molecules) from the probe image to create a final image comprising an enhanced target signal. In other words, the modified (i.e., transformed, adjusted, scaled, etc.) blank image is used in the subtracting step instead of the original blank image. In some embodiments, the enhanced target signal includes enhanced contrast. In some embodiments, the method includes displaying the final image on a display (e.g., a computer display). The final image may be saved to a memory and may be accessible by a user, for example, over a network. As such, the method provides improved signal detection in the presence of a background with tissue autofluorescence.

In some embodiments, the image metric is a ratio factor to account for intensity differences in background between the blank image and the probe image. Intensity differences can occur when image acquisition settings are different or from photobleaching during fluorophore excitation. To compensate for background intensity differences, the method includes determining a ratio factor that compares the overall background intensity of the probe image versus the blank image. First, the pixel locations of the probe are estimated. The probe locations in the probe image are estimated using, for example, the White Top Hat algorithm (Gonzalez & Woods, 2008, Digital Image Processing), bandpass filtering (Shenoi, 2006, Introduction to Digital Signal Processing and Filter Design), or any combination of suitable methods. After determining an estimated location of the target signals in the probe image, the pixels at the estimated probe locations are excluded from both the probe image and the blank image, resulting in background-pixel-only images (i.e., background-only images). In other words, the method includes removing the estimated location from the probe image to create a first background-only image and removing the estimated location from the blank image (background image) to create a second background-only image.

Following removal of the estimated probe locations from both images, the method includes determining a ratio factor. In other words, a statistical metric for both the probe-excluded blank image and the probe-excluded probe image is evaluated and incorporated into a ratio factor. The ratio factor is utilized in some embodiments to modify the background image to create an adjusted background image. In other words, modifying the background image to create an adjusted background image can include, in some embodiments, scaling the background image by the ratio factor.

In some embodiments, the at least one image metric is a ratio factor of the first background-only image and the second background-only image. For example, the ratio factor in some embodiments is a first intensity to a second intensity, with the first intensity is determined from the first background-only image and the second intensity is determined from the second background-only image. In some embodiments, the first and second intensities used in the ratio factor are statistical metrics such as a statistical mean, median, or a combination of both for any portion of (including all) the intensity values in an image.

In some embodiments, the first intensity is the mean of a plurality of pixel intensity values in the first background-only image and the second intensity is the mean of a plurality of pixel intensity values in the second background-only image. In some embodiments, the mean is of all the pixel intensity values in the image. In other embodiments, the first intensity is the median of a plurality of pixel intensity values in the first background-only image, and the second intensity is the median of a plurality of pixel intensity values in the second background-only image. In some embodiments, the median is of all the pixel intensity values in the image. In another embodiment, the first intensity is the mean of a central approximately 80% of all the pixel intensity values (i.e., excluding the approximate top 10% and the approximate bottom 10%) in the first background-only image, and the second intensity is the mean of a central approximately 80% of all the pixel intensity values in the second background-only image.

In some embodiments, the image metric is a multiplication factor to account for potential local intensity differences between the blank image and the probe image. In particular, the method includes determining the multiplication factor. In some embodiments, the multiplication factor is within a range of approximately 1.0 to approximately 1.2. In other embodiments, the multiplication factor is within a range of approximately 1.0 to approximately 1.1. The multiplication factor is utilized in some embodiments to modify the background image to create an adjusted background image. In other words, modifying the background image to create an adjusted background image can include, in some embodiments, scaling the background image by the multiplication factor.

In some embodiments, the image metric is a local maximum value transform. In particular, the method includes transforming the blank image with a local maximum value transform. Even after global image registration, there may remain local background pattern mismatches that from, for example, image acquisition at different focal planes, or samples not firmly attached to the supporting material (e.g., glass slides) and partially moving between imaging sessions. To resolve this issue, local mismatches are compensated with a transform. In the illustrated embodiment, for each pixel in the blank image (“pixel of interest”), a neighborhood of a pre-defined radius surrounding the pixel of interest is searched. The search process will find the pixel of maximum intensity, and this maximum intensity is assigned to that pixel of interest. This searching procedure is performed for each pixel of interest, searching its neighborhood in the original blank image, to form a transformed blank image. As explained in greater detail herein, the transformed blank image can be used instead of the original blank image in the later subtracting step. In some embodiments, the pre-defined radius (“match distance”) is adjustable.

In some embodiments, the pre-defined radius used in the local maximum valve transform is within a range of approximately 0 to approximately 5 pixels. In other words, the local maximum value transform includes a search radius within a range of approximately 0 to 5 pixels. A pre-defined radius of 0 pixels is utilized, for example, when there is no noticeable local background pattern mismatch. In some embodiments, the search area is simplified to reduce computational time by using eight angularly equally spaced lines (i.e., 45 degrees apart), each with a single-pixel width, radiating from the pixel of interest.

In some embodiments, the image metric is a block-matching transform. In particular, the method, in some embodiments, includes a step to transform the blank image with a block-matching transform. In some embodiments, the block-matching transform is used in place of the local maximum value transform to resolve the issue of local mismatches. In some embodiments, a block (“block of interest”) is used with a pre-defined block size (e.g., a 3-pixel-by-3-pixel block). Each block in the blank image is compared with blocks of the same size in the probe image in nearby locations (i.e., within a pre-defined block search size). The search determines the nearby block that is most similar to the block of interest. A similarity metric is utilized to measure the similarity of the blocks, and the searched nearby block with the highest similarity metric is determined to be the target block. Then, the block of interest is moved to the corresponding location of the target block. In some embodiments, the similarity metric is a mean absolute difference, a sum of absolute difference, a mean squared difference, or a sum of squared difference, wherein the differences are the pixel intensity differences between the two blocks being compared. As such, the block-matching transform is performed for each block of interest, searching its corresponding neighborhood in the probe image and moving its location accordingly, to form a transformed blank image. In some embodiments, this transformed blank image is used instead of the original blank image in later subtracting steps.

In some embodiments, the pre-defined block size and the pre-defined block search size are adjustable. In some embodiments, the pre-defined block size used in the block-matching transform is within a range of approximately 1 to approximately 10 pixels. In other words, the block-matching transform includes a block size within a range of approximately 1 to 10 pixels. In some embodiments, the pre-defined block search size used in the block matching transform is within a range of approximately 1 to approximately 10 pixels. In other words, the block-matching transform includes a block search size within a range of approximately 1 to 10 pixels.

In some embodiments, the method for enhancing detection of a target includes any combination of the steps described herein, in various orders. In some embodiments, steps may be omitted. Further, the order of the steps may be reversed, altered, or performed simultaneously.

In at least one embodiment, the electronic-based aspects of the method may be implemented in software (e.g., stored on non-transitory computer-readable medium) executable by a computer with one or more processing units, such as a microprocessor and/or application specific integrated circuits (“ASICs”). Some embodiments may include hardware, software, and electronic components or modules. As such, it should be noted that a plurality of hardware and software-based devices, as well as a plurality of different structural components, may be utilized to implement the embodiments.

### Experimental Materials and Methods

Tissue collection. Mouse brain sections (5 um thick) were collected from a fresh-frozen BL6 mouse brain from a 1 month old mouse and a 12-month-old mouse. The tissues were embedded in OCT, snap-frozen, and sectioned coronally from the brain block with a cryotome. Brain sections were fixed in 10% NBF and dehydrated in Ethanol before the assay.

Target probe selections, synthesis, and formulation. One hundred mouse genes were selected as the target of transcriptomic profiling. These genes were examined for their spatial expression pattern in the mouse brains, their expression levels, and their specificity for brain cell types using single-cell RNAseq datasets from public sc-RNA seq data deposits. Multiplex and Hiplex assays were also performed on the coronal mouse brain sections to further verify the spatial expression patterns of some high-expression genes. The goal of these screening steps was to avoid any spatial crowding in the final FISH images, which would otherwise defeat the decoding process.

All target probes were designed by ACD probe design team and were checked to ensure no potential cross-reactivity to other RNA species. All DNA oligo probes were formulated at 10 uM concentration and were pooled in ACD hybridization buffer A at 1:500 dilution. 100 target probes were designed and synthesized for the first cycle, and another set of 100 target probes for the second cycle of the assay.

Coding Scheme. The assay is built on a codebook that is composed of 10 fundamental codes. Each fundamental code has 5 bits and two fluorescence signals (e.g., ‘10100’ has an AF488 signal and a Dy594 signal). A pool of 5 unique fluorophores was used to generate various combinations of two fluorophores, which include: AF488, Dy550, Dy650, Dy594, and AF750. The resultant 5-bit codes are listed below:

TABLE 4
The list of the 10 fundamental codes used in an
exemplary 100plex Ultraplex assay. Each code has
5 bits and contains two fluorescence signals.

| | Hamming | | | | | |
|---|---|---|---|---|---|---|
| S.N. | Weight | AF488 | Dy550 | Dy594 | Dy650 | AF750 |
| No. 1 | 2-fluorophore | 0 | 0 | 0 | 1 | 1 |
| No. 2 | 2-fluorophore | 0 | 0 | 1 | 0 | 1 |
| No. 3 | 2-fluorophore | 0 | 0 | 1 | 1 | 0 |
| No. 4 | 2-fluorophore | 0 | 1 | 0 | 0 | 1 |
| No. 5 | 2-fluorophore | 0 | 1 | 0 | 1 | 0 |
| No. 6 | 2-fluorophore | 0 | 1 | 1 | 0 | 0 |
| No. 7 | 2-fluorophore | 1 | 0 | 0 | 0 | 1 |
| No. 8 | 2-fluorophore | 1 | 0 | 0 | 1 | 0 |
| No. 9 | 2-fluorophore | 1 | 0 | 1 | 0 | 0 |
| No. 10 | 2-fluorophore | 1 | 1 | 0 | 0 | 0 |

To reach the 100 plex analytic capacity, the 5-bit codes were expanded to create a new codebook with 100 ‘10-bit’ codes. This was accomplished by running two barcoding cycles, where the primary 5-bit code is randomly paired with another 5-bit code from the list above (Table 4). Each 10-bit code is unique in the codebook and can be assigned to a gene target. This book of 100 codes is provided in Table 5.

TABLE 5
Codebook for an exemplary 100plex Ultraplex assay. Each
code has 10 bits and contains four fluorescence signals.

| | | | | | | | | | | | cycle- | cycle- |
|---|---|---|---|---|---|---|---|---|---|---|---|---|
| | cycle-1 code | | | | | cycle-2 code | | | | | 1 Tree | 2 tree |
| No. 1 | 0 | 0 | 0 | 1 | 1 | 0 | 0 | 0 | 1 | 1 | T-a | T-a |
| No. 2 | 0 | 0 | 0 | 1 | 1 | 0 | 0 | 1 | 0 | 1 | T-a | T-b |
| No. 3 | 0 | 0 | 0 | 1 | 1 | 0 | 0 | 1 | 1 | 0 | T-a | T-c |
| No. 4 | 0 | 0 | 0 | 1 | 1 | 0 | 1 | 0 | 0 | 1 | T-a | T-d |
| No. 5 | 0 | 0 | 0 | 1 | 1 | 0 | 1 | 0 | 1 | 0 | T-a | T-e |
| No. 6 | 0 | 0 | 0 | 1 | 1 | 0 | 1 | 1 | 0 | 0 | T-a | T-f |
| No. 7 | 0 | 0 | 0 | 1 | 1 | 1 | 0 | 0 | 0 | 1 | T-a | T-g |
| No. 8 | 0 | 0 | 0 | 1 | 1 | 1 | 0 | 0 | 1 | 0 | T-a | T-h |
| No. 9 | 0 | 0 | 0 | 1 | 1 | 1 | 0 | 1 | 0 | 0 | T-a | T-i |
| No. 10 | 0 | 0 | 0 | 1 | 1 | 1 | 1 | 0 | 0 | 0 | T-a | T-j |
| No. 11 | 0 | 0 | 1 | 0 | 1 | 0 | 0 | 0 | 1 | 1 | T-b | T-a |
| No. 12 | 0 | 0 | 1 | 0 | 1 | 0 | 0 | 1 | 0 | 1 | T-b | T-b |
| No. 13 | 0 | 0 | 1 | 0 | 1 | 0 | 0 | 1 | 1 | 0 | T-b | T-c |
| No. 14 | 0 | 0 | 1 | 0 | 1 | 0 | 1 | 0 | 0 | 1 | T-b | T-d |
| No. 15 | 0 | 0 | 1 | 0 | 1 | 0 | 1 | 0 | 1 | 0 | T-b | T-e |
| No. 16 | 0 | 0 | 1 | 0 | 1 | 0 | 1 | 1 | 0 | 0 | T-b | T-f |
| No. 17 | 0 | 0 | 1 | 0 | 1 | 1 | 0 | 0 | 0 | 1 | T-b | T-g |
| No. 18 | 0 | 0 | 1 | 0 | 1 | 1 | 0 | 0 | 1 | 0 | T-b | T-h |
| No. 19 | 0 | 0 | 1 | 0 | 1 | 1 | 0 | 1 | 0 | 0 | T-b | T-i |
| No. 20 | 0 | 0 | 1 | 0 | 1 | 1 | 1 | 0 | 0 | 0 | T-b | T-j |
| No. 21 | 0 | 0 | 1 | 1 | 0 | 0 | 0 | 0 | 1 | 1 | T-c | T-a |
| No. 22 | 0 | 0 | 1 | 1 | 0 | 0 | 0 | 1 | 0 | 1 | T-c | T-b |
| No. 23 | 0 | 0 | 1 | 1 | 0 | 0 | 0 | 1 | 1 | 0 | T-c | T-c |
| No. 24 | 0 | 0 | 1 | 1 | 0 | 0 | 1 | 0 | 0 | 1 | T-c | T-d |
| No. 25 | 0 | 0 | 1 | 1 | 0 | 0 | 1 | 0 | 1 | 0 | T-c | T-e |
| No. 26 | 0 | 0 | 1 | 1 | 0 | 0 | 1 | 1 | 0 | 0 | T-c | T-f |
| No. 27 | 0 | 0 | 1 | 1 | 0 | 1 | 0 | 0 | 0 | 1 | T-c | T-g |
| No. 28 | 0 | 0 | 1 | 1 | 0 | 1 | 0 | 0 | 1 | 0 | T-c | T-h |
| No. 29 | 0 | 0 | 1 | 1 | 0 | 1 | 0 | 1 | 0 | 0 | T-c | T-i |
| No. 30 | 0 | 0 | 1 | 1 | 0 | 1 | 1 | 0 | 0 | 0 | T-c | T-j |
| No. 31 | 0 | 1 | 0 | 0 | 1 | 0 | 0 | 0 | 1 | 1 | T-d | T-a |
| No. 32 | 0 | 1 | 0 | 0 | 1 | 0 | 0 | 1 | 0 | 1 | T-d | T-b |
| No. 33 | 0 | 1 | 0 | 0 | 1 | 0 | 0 | 1 | 1 | 0 | T-d | T-c |
| No. 34 | 0 | 1 | 0 | 0 | 1 | 0 | 1 | 0 | 0 | 1 | T-d | T-d |
| No. 35 | 0 | 1 | 0 | 0 | 1 | 0 | 1 | 0 | 1 | 0 | T-d | T-e |
| No. 36 | 0 | 1 | 0 | 0 | 1 | 0 | 1 | 1 | 0 | 0 | T-d | T-f |
| No. 37 | 0 | 1 | 0 | 0 | 1 | 1 | 0 | 0 | 0 | 1 | T-d | T-g |
| No. 38 | 0 | 1 | 0 | 0 | 1 | 1 | 0 | 0 | 1 | 0 | T-d | T-h |
| No. 39 | 0 | 1 | 0 | 0 | 1 | 1 | 0 | 1 | 0 | 0 | T-d | T-i |
| No. 40 | 0 | 1 | 0 | 0 | 1 | 1 | 1 | 0 | 0 | 0 | T-d | T-j |
| No. 41 | 0 | 1 | 0 | 1 | 0 | 0 | 0 | 0 | 1 | 1 | T-e | T-a |
| No. 42 | 0 | 1 | 0 | 1 | 0 | 0 | 0 | 1 | 0 | 1 | T-e | T-b |
| No. 43 | 0 | 1 | 0 | 1 | 0 | 0 | 0 | 1 | 1 | 0 | T-e | T-c |
| No. 44 | 0 | 1 | 0 | 1 | 0 | 0 | 1 | 0 | 0 | 1 | T-e | T-d |
| No. 45 | 0 | 1 | 0 | 1 | 0 | 0 | 1 | 0 | 1 | 0 | T-e | T-e |
| No. 46 | 0 | 1 | 0 | 1 | 0 | 0 | 1 | 1 | 0 | 0 | T-e | T-f |
| No. 47 | 0 | 1 | 0 | 1 | 0 | 1 | 0 | 0 | 0 | 1 | T-e | T-g |
| No. 48 | 0 | 1 | 0 | 1 | 0 | 1 | 0 | 0 | 1 | 0 | T-e | T-h |
| No. 49 | 0 | 1 | 0 | 1 | 0 | 1 | 0 | 1 | 0 | 0 | T-e | T-i |
| No. 50 | 0 | 1 | 0 | 1 | 0 | 1 | 1 | 0 | 0 | 0 | T-e | T-j |
| No. 51 | 0 | 1 | 1 | 0 | 0 | 0 | 0 | 0 | 1 | 1 | T-f | T-a |
| No. 52 | 0 | 1 | 1 | 0 | 0 | 0 | 0 | 1 | 0 | 1 | T-f | T-b |
| No. 53 | 0 | 1 | 1 | 0 | 0 | 0 | 0 | 1 | 1 | 0 | T-f | T-c |
| No. 54 | 0 | 1 | 1 | 0 | 0 | 0 | 1 | 0 | 0 | 1 | T-f | T-d |
| No. 55 | 0 | 1 | 1 | 0 | 0 | 0 | 1 | 0 | 1 | 0 | T-f | T-e |
| No. 56 | 0 | 1 | 1 | 0 | 0 | 0 | 1 | 1 | 0 | 0 | T-f | T-f |
| No. 57 | 0 | 1 | 1 | 0 | 0 | 1 | 0 | 0 | 0 | 1 | T-f | T-g |
| No. 58 | 0 | 1 | 1 | 0 | 0 | 1 | 0 | 0 | 1 | 0 | T-f | T-h |
| No. 59 | 0 | 1 | 1 | 0 | 0 | 1 | 0 | 1 | 0 | 0 | T-f | T-i |
| No. 60 | 0 | 1 | 1 | 0 | 0 | 1 | 1 | 0 | 0 | 0 | T-f | T-j |
| No. 61 | 1 | 0 | 0 | 1 | 0 | 0 | 0 | 0 | 1 | 1 | T-h | T-a |
| No. 62 | 1 | 0 | 0 | 1 | 0 | 0 | 0 | 1 | 0 | 1 | T-h | T-b |
| No. 63 | 1 | 0 | 0 | 1 | 0 | 0 | 0 | 1 | 1 | 0 | T-h | T-c |
| No. 64 | 1 | 0 | 0 | 1 | 0 | 0 | 1 | 0 | 0 | 1 | T-h | T-d |
| No. 65 | 1 | 0 | 0 | 1 | 0 | 0 | 1 | 0 | 1 | 0 | T-h | T-e |
| No. 66 | 1 | 0 | 0 | 1 | 0 | 0 | 1 | 1 | 0 | 0 | T-h | T-f |
| No. 67 | 1 | 0 | 0 | 1 | 0 | 1 | 0 | 0 | 0 | 1 | T-h | T-g |
| No. 68 | 1 | 0 | 0 | 1 | 0 | 1 | 0 | 0 | 1 | 0 | T-h | T-h |
| No. 69 | 1 | 0 | 0 | 1 | 0 | 1 | 0 | 1 | 0 | 0 | T-h | T-i |
| No. 70 | 1 | 0 | 0 | 1 | 0 | 1 | 1 | 0 | 0 | 0 | T-h | T-j |
| No. 71 | 1 | 0 | 0 | 0 | 1 | 0 | 0 | 0 | 1 | 1 | T-g | T-a |
| No. 72 | 1 | 0 | 0 | 0 | 1 | 0 | 0 | 1 | 0 | 1 | T-g | T-b |
| No. 73 | 1 | 0 | 0 | 0 | 1 | 0 | 0 | 1 | 1 | 0 | T-g | T-c |
| No. 74 | 1 | 0 | 0 | 0 | 1 | 0 | 1 | 0 | 0 | 1 | T-g | T-d |
| No. 75 | 1 | 0 | 0 | 0 | 1 | 0 | 1 | 0 | 1 | 0 | T-g | T-e |
| No. 76 | 1 | 0 | 0 | 0 | 1 | 0 | 1 | 1 | 0 | 0 | T-g | T-f |
| No. 77 | 1 | 0 | 0 | 0 | 1 | 1 | 0 | 0 | 0 | 1 | T-g | T-g |
| No. 78 | 1 | 0 | 0 | 0 | 1 | 1 | 0 | 0 | 1 | 0 | T-g | T-h |
| No. 79 | 1 | 0 | 0 | 0 | 1 | 1 | 0 | 1 | 0 | 0 | T-g | T-i |
| No. 80 | 1 | 0 | 0 | 0 | 1 | 1 | 1 | 0 | 0 | 0 | T-g | T-j |
| No. 81 | 1 | 0 | 1 | 0 | 0 | 0 | 0 | 0 | 1 | 1 | T-i | T-a |
| No. 82 | 1 | 0 | 1 | 0 | 0 | 0 | 0 | 1 | 0 | 1 | T-i | T-b |
| No. 83 | 1 | 0 | 1 | 0 | 0 | 0 | 0 | 1 | 1 | 0 | T-i | T-c |
| No. 84 | 1 | 0 | 1 | 0 | 0 | 0 | 1 | 0 | 0 | 1 | T-i | T-d |
| No. 85 | 1 | 0 | 1 | 0 | 0 | 0 | 1 | 0 | 1 | 0 | T-i | T-e |
| No. 86 | 1 | 0 | 1 | 0 | 0 | 0 | 1 | 1 | 0 | 0 | T-i | T-f |
| No. 87 | 1 | 0 | 1 | 0 | 0 | 1 | 0 | 0 | 0 | 1 | T-i | T-g |
| No. 88 | 1 | 0 | 1 | 0 | 0 | 1 | 0 | 0 | 1 | 0 | T-i | T-h |
| No. 89 | 1 | 0 | 1 | 0 | 0 | 1 | 0 | 1 | 0 | 0 | T-i | T-i |
| No. 90 | 1 | 0 | 1 | 0 | 0 | 1 | 1 | 0 | 0 | 0 | T-i | T-j |
| No. 91 | 1 | 1 | 0 | 0 | 0 | 0 | 0 | 0 | 1 | 1 | T-j | T-a |
| No. 92 | 1 | 1 | 0 | 0 | 0 | 0 | 0 | 1 | 0 | 1 | T-j | T-b |
| No. 93 | 1 | 1 | 0 | 0 | 0 | 0 | 0 | 1 | 1 | 0 | T-j | T-c |
| No. 94 | 1 | 1 | 0 | 0 | 0 | 0 | 1 | 0 | 0 | 1 | T-j | T-d |
| No. 95 | 1 | 1 | 0 | 0 | 0 | 0 | 1 | 0 | 1 | 0 | T-j | T-e |
| No. 96 | 1 | 1 | 0 | 0 | 0 | 0 | 1 | 1 | 0 | 0 | T-j | T-f |
| No. 97 | 1 | 1 | 0 | 0 | 0 | 1 | 0 | 0 | 0 | 1 | T-j | T-g |
| No. 98 | 1 | 1 | 0 | 0 | 0 | 1 | 0 | 0 | 1 | 0 | T-j | T-h |
| No. 99 | 1 | 1 | 0 | 0 | 0 | 1 | 0 | 1 | 0 | 0 | T-j | T-i |
| No. 100 | 1 | 1 | 0 | 0 | 0 | 1 | 1 | 0 | 0 | 0 | T-j | T-j |

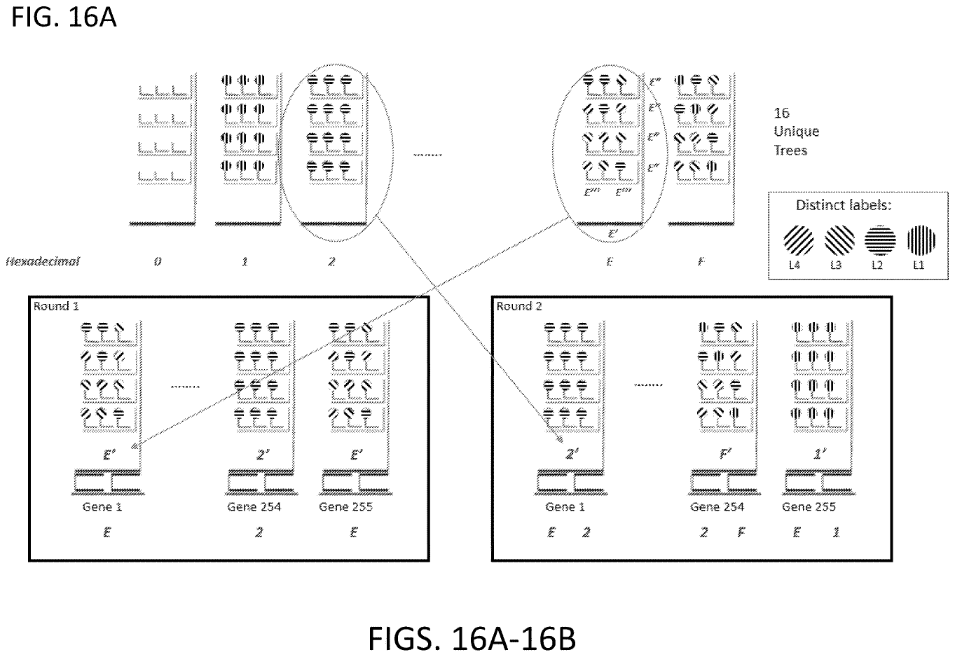

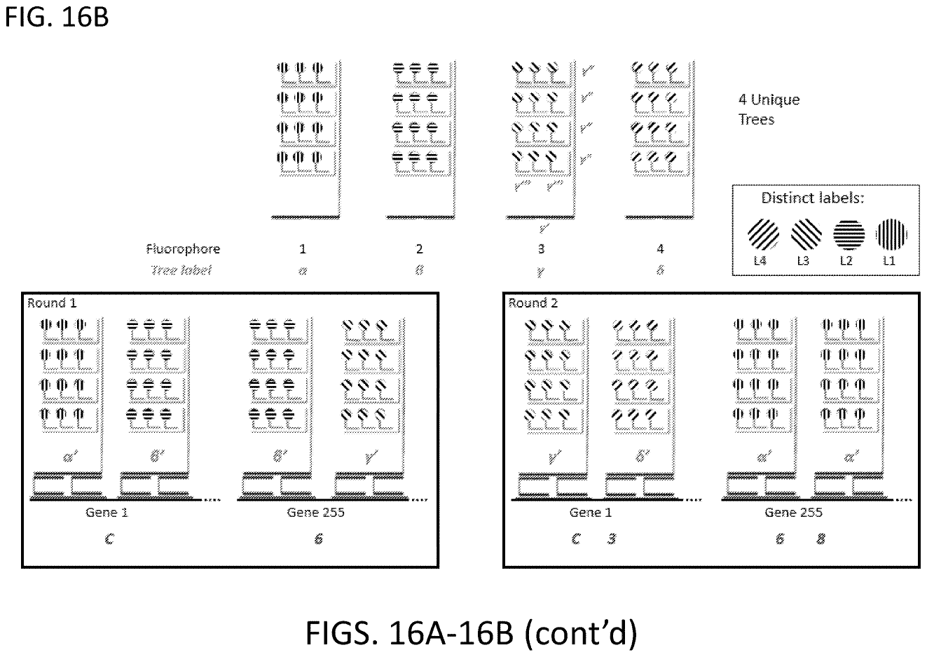

RNA molecule labeling. To encode the RNA molecules with corresponding codes, each RNA transcript was first hybridized a specific double-Z target probe designed by ACD. The target probe was then annealed with 3 tiers of branching DNA oligos (i.e., AMP1, AMP2, and AMP3). The third AMP oligo contained a unique binding sequence that compliments the corresponding fluorophore-conjugated label oligos (label probe, or LP). The development of signal generating configuration was completed upon incubating the branching DNA structure with its corresponding label probes. For each RNA species, the branching DNA structure it hybridized to was annealed with its complementary label probes that were conjugated with either fluorophore A or fluorophore B (50%:50% LP-A/LP-B in the final solution), as pre-determined in the codebook (Table 5). This allows the RNA molecule to emit two fluorescence signals under a fluorescent microscope, and therefore display a unique 5-bit code in the acquired multi-spectral image. All 100 RNA targets included in the panel were encoded twice through two independent barcoding cycles. The first cycle of barcoding provides the first 5 bits of the code, and the second cycle provides the last 5 bits of the code. In each cycle, all 100 genes were simultaneously labeled with their corresponding 5-bit codes, which does not require any specific temporal barcoding order (FIGS. 16A-16B). In addition, the two cycles were independent of each other. Each barcoding cycle can be performed either first or last. In each cycle, any prior branching DNA structures were disrupted and stripped off from the RNA molecules using the HiplexUP reagent, and the RNA molecules were subject to a new signal generation configuration, beginning with a new target probe hybridization.

This assay has been performed on both Leica BondRx system and with a streamlined manual protocol. Both approaches resulted in excellent positive signals and very clean background.

Protease pretreatment for target retrieval. For the Ultraplex assay, the ACD PT3, PT4 or Sigma ready-to-use pepsin solution (cat No. R2283) were used, depending on the tissue types, tissue processing methods, and extent of formalin fixation. For example, in the Ultraplex assay with mouse brain FFPE tissue, the tissue was incubated with 300 uL of pepsin at room temperature for 10 mins. Pepsin offers some unique benefits over the PT3 or PT4, including reduction of autofluorescent background in the FFPE tissues, removal of >90% red blood cells, and removal of non-specific signals in the Ultraplex assay, which all contribute to a higher signal-to-noise ratio.

Image analysis. The stained tissue slides were imaged by a Zeiss Axio Imager Z2 epifluorescent microscope. For each tissue, an xyz scan was conducted in the entire tissue. Final fluorescent microscopic images underwent a series of pre-processing steps: image correction for chromatic errors and optical field shading, tile image registration for cycle1 and cycle2 scans, tile stitching to a large tissue image. The preprocessed whole-slide images were then imported into an advanced decoding pipeline that are developed with deep learning by ACD image analysis team to extract the spatial transcriptomics information from the test tissues. Final assay outputs from the pipeline include: a cell×feature matrix, cell segmentation boundaries, field of view ID, the x and y coordinates of all detected transcripts in the tissue, and x and y coordinates of the centroids of each cell in the tissue.

### EXAMPLES

### Example 1

The experiments below used the “code book” set forth in Table 6, using the indicated labeling schemes and corresponding alphanumeric code in each round of labeling and detection.

TABLE 6
Code book based on four distinct labels to generate
independent identifiers of various targets
in the experiments disclosed herein.

| Presence of labels (“X” present, “—” absent) | | | | |
|---|---|---|---|---|
| L1 | | | L4 | |
| (Alexa | L2 | L3 | Alexa | |
| Fluor 488/ | Dylight | Dylight | Fluor 750/ | Alphanumeric |
| FITC) | 550/Cy3 | 650/Cy5 | Cy7 | Code |
| — | — | — | — | 0 |
| X | — | — | — | 1 |
| — | X | — | — | 2 |
| — | — | X | — | 3 |
| — | — | — | X | 4 |
| X | X | — | — | 5 |
| X | — | X | — | 6 |
| X | — | — | X | 7 |
| — | X | X | — | 8 |
| — | X | — | X | 9 |
| — | — | X | X | A |
| X | X | X | — | B |
| X | — | X | X | C |
| X | X | — | X | D |
| — | X | X | X | E |
| X | X | X | X | F |

A single gene (RNA transcript), Ube, was detected in a two-cycle experiment according to the methods disclosed herein. In each round of labeling, the independent identifier was based on a single fluorescent label, corresponding to Code 3 in Table 1a.

Samples (fresh-frozen Balb/c mouse brain sections from Acepix) were fixed 10% neutral buffered formalin (NBF) for 1 hour at room temperature, subjected to serial dehydration in 50%, 80%, and 100% ethanol, and then subjected to protease-IV digestion for 30 minutes at room temperature. For the first round of labeling, target probe sets were hybridized for 2 hours at 40 °C, followed by hybridization of amplification reagents, and incubation with label probes (based on Dy650) for 15 minutes at 40 °C. Following DAPI counterstaining, mounting, and imaging (AkoyaPolaris microscope, 40×), the fluorophores from the label probes were cleaved, and the amplification trees were removed using the HiplexUP reagent (Advanced Cell Diagnostics, Inc.) (3 incubations at 5 minutes each, room temperature). A second round of target probe sets were then hybridized for 2 hours at 40 °C, followed by hybridization of amplification reagents, incubation with label probes (based on Dy650) for 15 minutes at 40 °C, DAPI counterstaining, mounting, and imaging (Akoya Polaris microscope, 40×). Standard washing steps were used between the main hybridization steps.

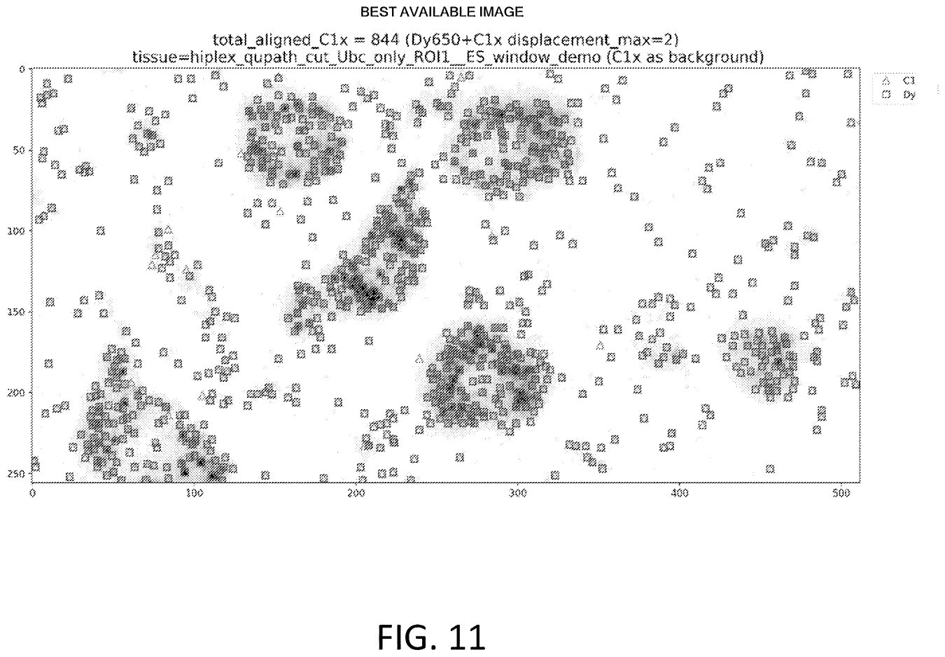

A resulting combined image from the two rounds of detection is shown in FIG. 11. The signals detected in the first round are indicated by triangles, and signals detected in the second round are indicated by squares. Locations with overlapping triangles and squares are the locations where the target gene, Ube, was detected.

### Example 2

Three genes (RNA transcripts Gadph, Sdha, and Ubc) were detected in a two-cycle experiment according to the methods disclosed herein. Each target was assigned the same first independent identifier based on a single fluorescent label (Code 3, see Table 6), and each target was assigned a different second independent identifier based on a single fluorescent label (Codes 1, 2, and 3 respectively, see Table 6).

Samples (fresh-frozen Balb/c mouse brain sections from Acepix) were fixed 10% neutral buffered formalin (NBF) for 1 hour at room temperature, subjected to serial dehydration in 50%, 80%, and 100% ethanol, and then subjected to protease-IV digestion for 30 minutes at room temperature. For the first round of labeling, target probe sets were hybridized for 2 hours at 40 °C, followed by hybridization of amplification reagents, and incubation with label probes (based on Dy650) for 15 minutes at 40 °C. Following DAPI counterstaining, mounting, and imaging (AkoyaPolaris microscope, 40×), the fluorophores from the label probes were cleaved, and the amplification trees were removed using the HiplexUP reagent (Advanced Cell Diagnostics, Newark, CA) (3 incubations at 5 minutes each, room temperature). A second round of target probe sets were then hybridized for 2 hours at 40 °C, followed by hybridization of amplification reagents, incubation with label probes (based on Dy650, Dy550, and AF488) for 15 minutes at 40 °C, DAPI counterstaining, mounting, and imaging (Akoya Polaris microscope, 40×). Standard washing steps were used between the main hybridization steps.

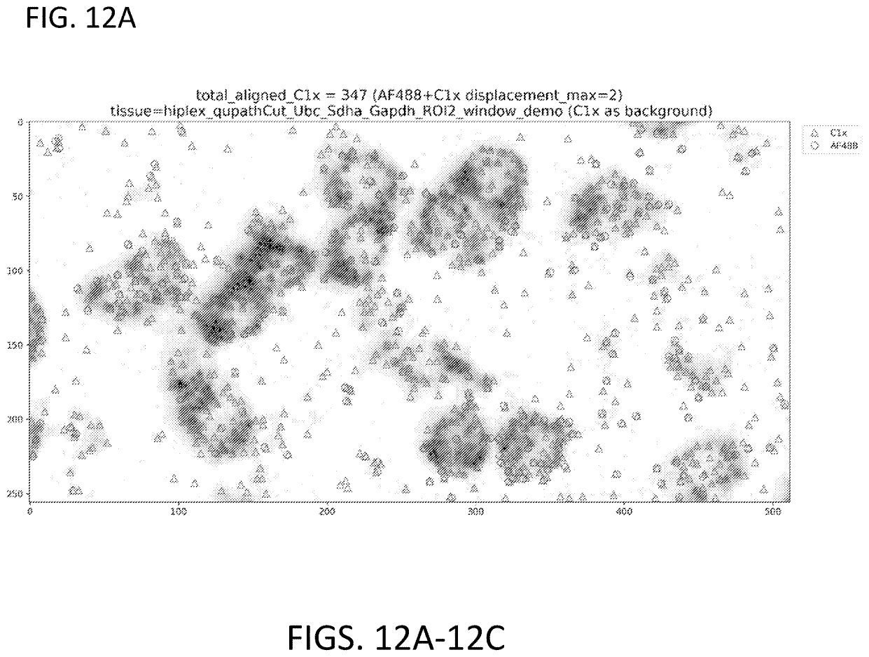

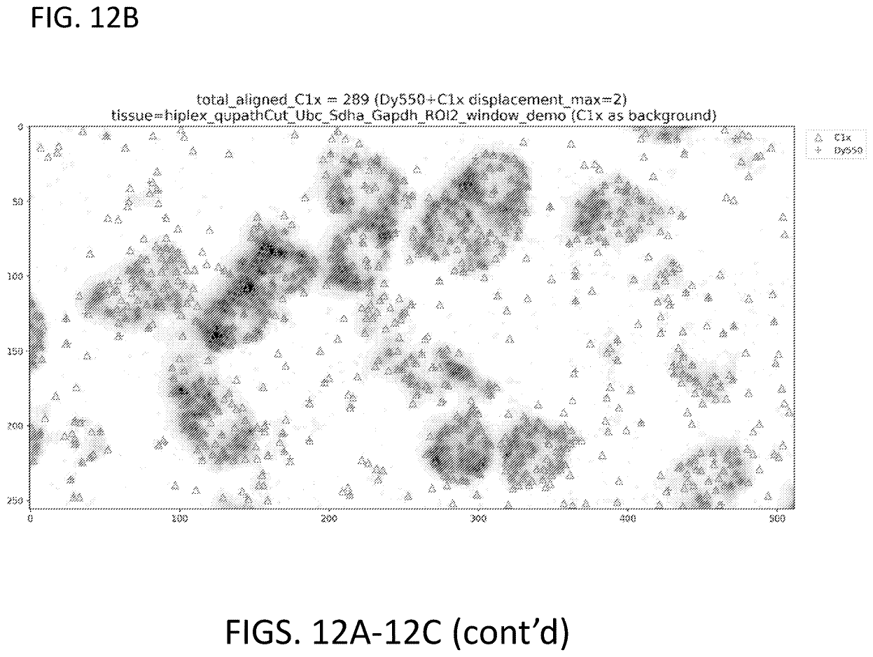

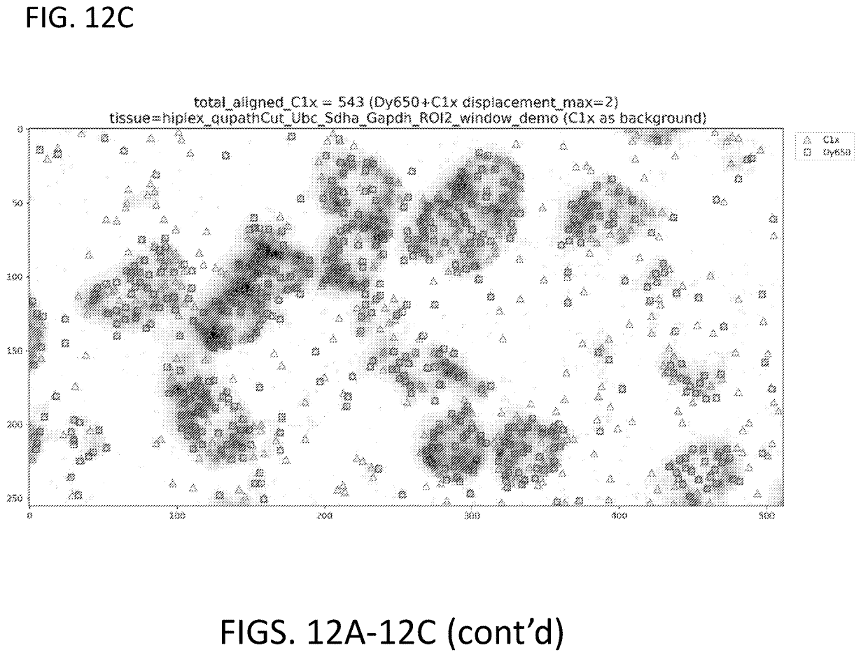

FIG. 12A shows detection of Gadph, FIG. 12B shows detection of Sdha, and FIG. 12C shows detection of Ubc. In each image, the signals detected in the first cycle (i.e., all three targets), are shown in triangles. In FIG. 12A, signals detected in the second cycle are indicated by circles, and locations with overlapping triangles and circles are the locations where the combined first and second identifiers (i.e., 31) show that Gadph was detected. In FIG. 12B, signals detected in the second cycle are indicated by crosses, and locations with overlapping triangles and crosses are the locations where the combined first and second identifiers (i.e., 32) show that Sdha was detected. In FIG. 12C, signals detected in the second cycle are indicated by squares, and locations with overlapping triangles and squares are the locations where the combined first and second identifiers (i.e., 33) show that Ubc was detected.

### Example 3

Experiments were conducted to demonstrate that target nucleic acids can be detected using the disclosed methods independent of the order in which the first and second independent identifiers are obtained. The human peptidylprolyl isomerase B (Hs-PPIB) gene transcript was detected in two separate two-cycle experiments, each using two fluorescent labels in each round of detection. In this experiment, L1=Alexa Fluor 488, L2=Dylight 550, L3=Dylight 650, and L4=Alexa Fluor 750 (see Table 6). In the first experiment, the first independent identifier was assigned Code A (L3, L4) and the second independent identifier was assigned Code 8 (L2, L3). In the second experiment, the identifiers were reversed, with the first independent identifier being assigned Code 8 (L2, L3) and the second independent identifier being assigned Code A (L3, L4).

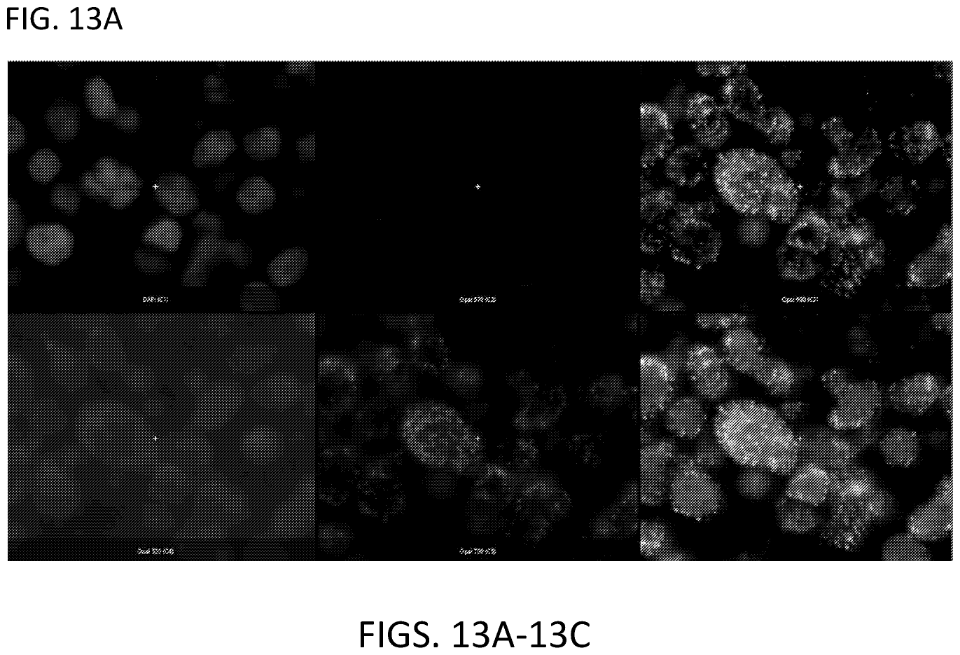

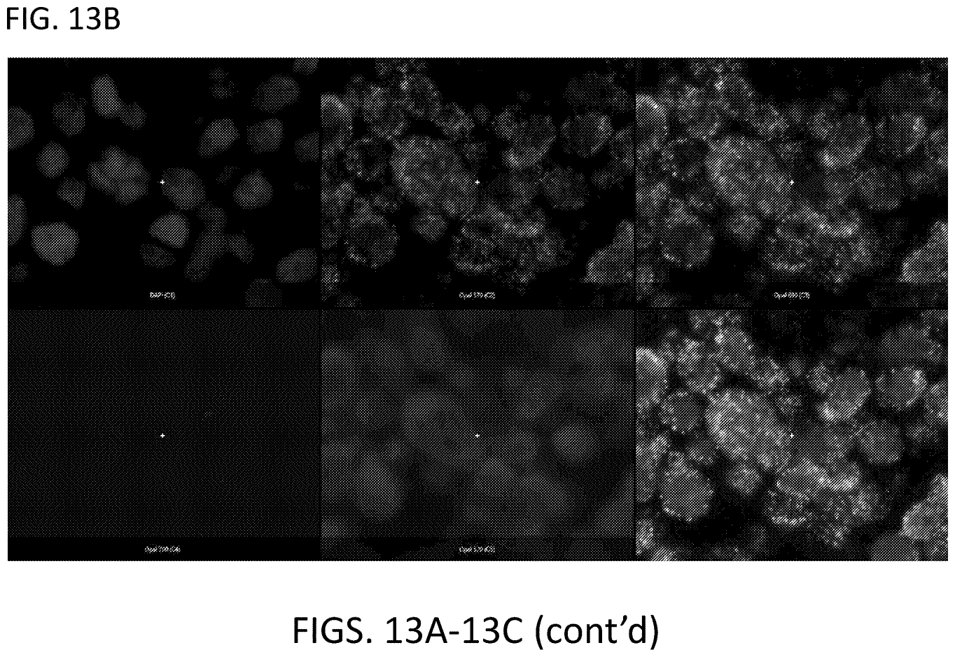

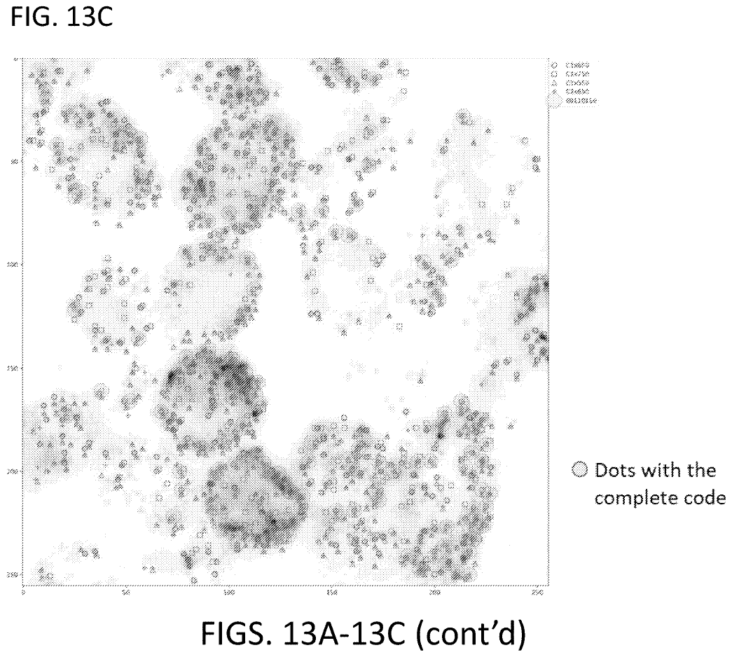

In the first experiment, following standard pretreatment of an FFPE HeLa cell pellet (including baking, deparaffinization, heat-induced epitope retrieval, and protease treatment), target probes were hybridized to the target for 2 hours at 40 °C, followed by treatment with amplification reagents for 30 minutes each at 40 °C, and then with the appropriate label probes for 30 minutes at 40 °C. Following DAPI counterstaining, mounting, and imaging (Akoya Polaris microscope, 40×, images shown in FIG. 13A), the labels were cleaved and the signal-generating complexes were removed using a cleaving reagent and HiplexUP reagent (Advanced Cell Diagnostics, Newark, CA), respectively. The second round of labeling and detection was conducted in a manner similar to the first round. Standard washing steps were used between the main hybridization steps. Images are shown in FIG. 13B, and an overlay of the signals detected in the first and second rounds of labeling and imaging is shown in FIG. 13C, with shaded circles showing locations in which the full target identifier (A8) was detected.

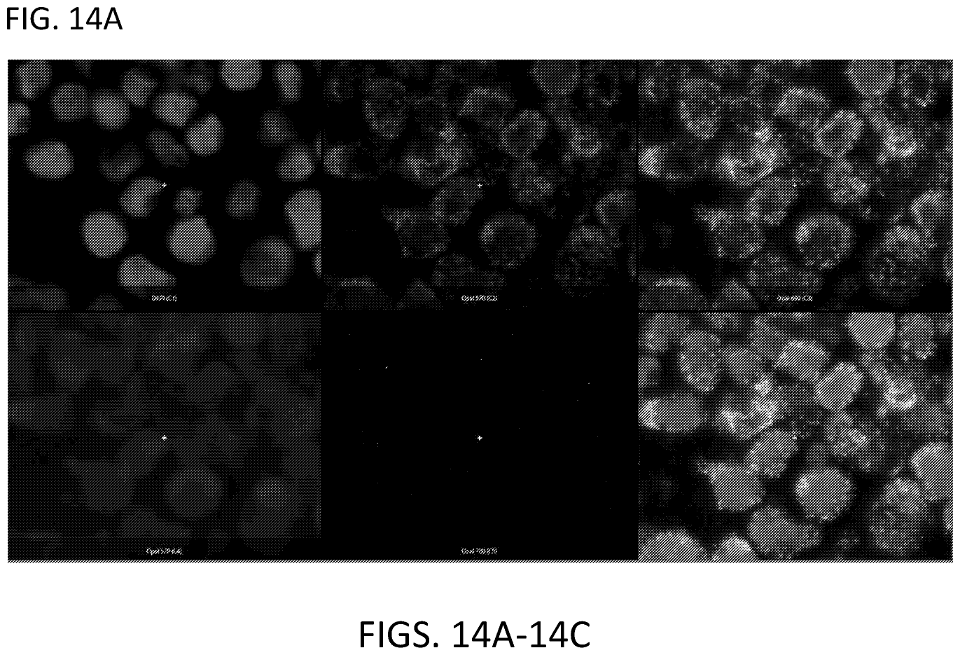

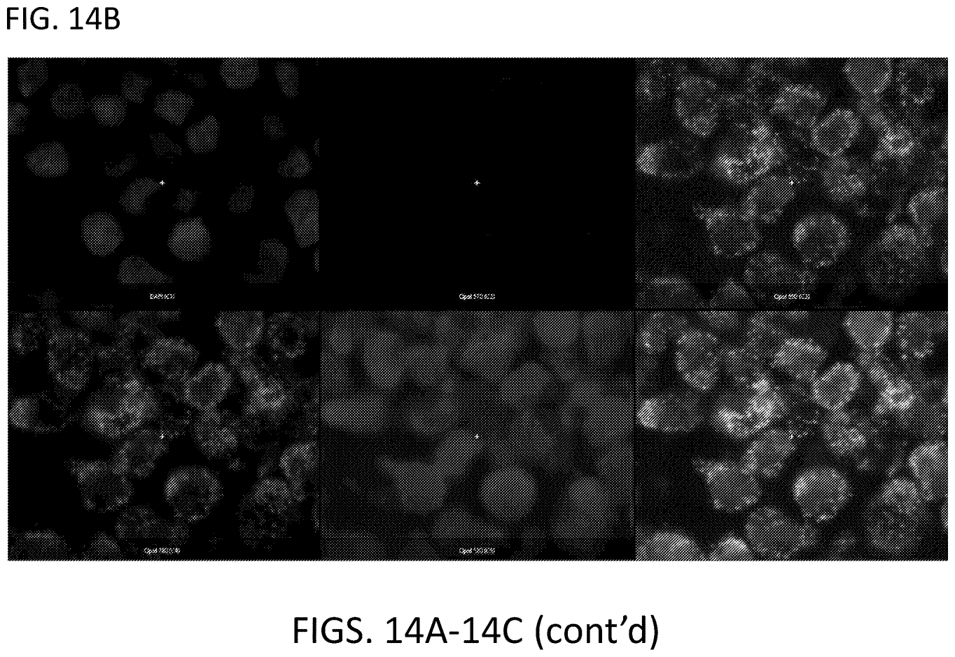

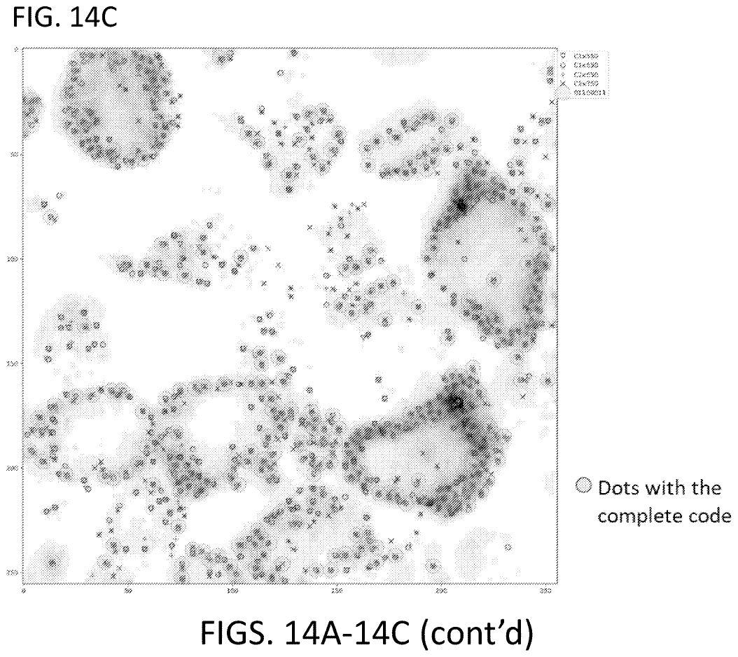

The second experiment was conducted with a reversed coding approach. Images from each round of labeling are shown in FIGS. 14A and 14B, and an overlay is shown in FIG. 14C.

These results demonstrate that the disclosed methods are not dependent on any particular temporal order of signals, but rather the first and second independent identifiers can be detected in either order with equal efficiency, and result in the same level of target detection.

### Example 4

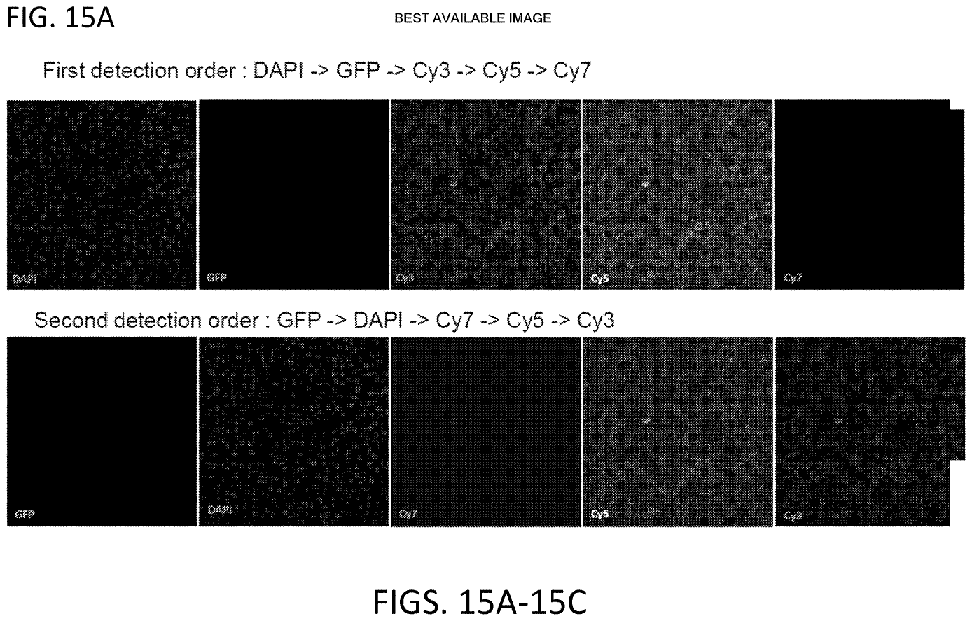

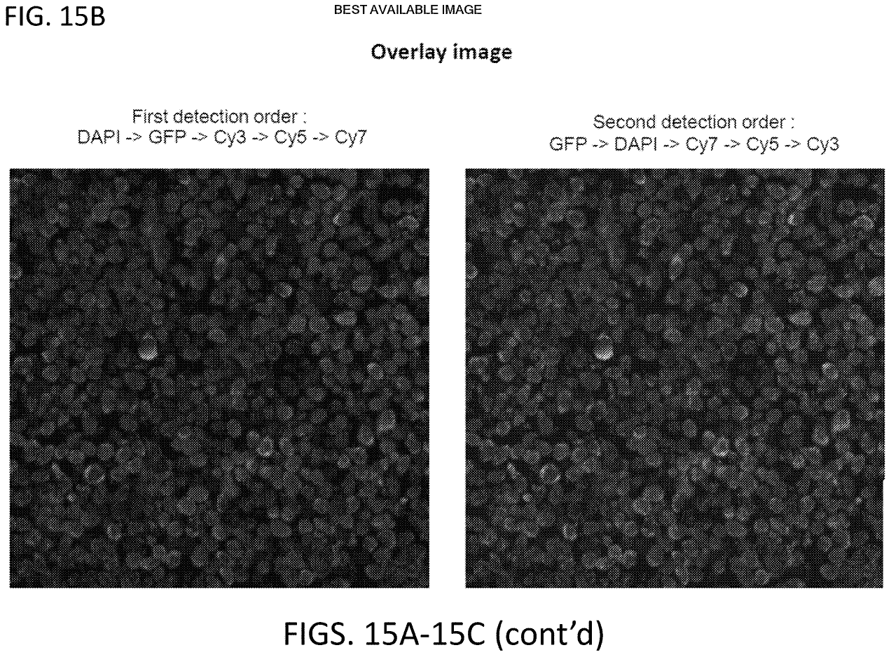

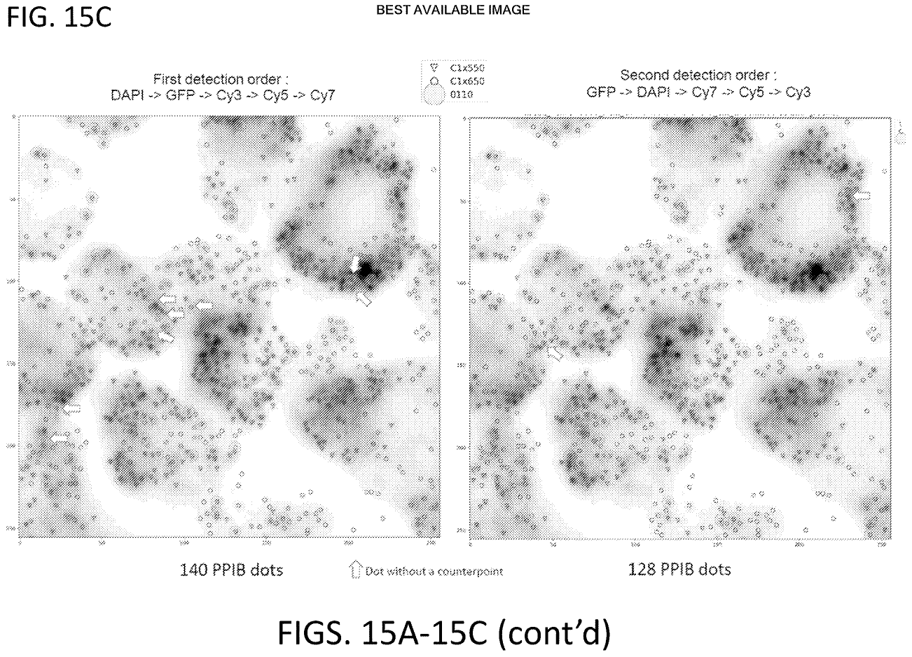

Experiments were conducted to demonstrate that the individual signals from different labels in an SGC used in methods disclosed herein can be detected in any order to generate each independent identifier. Following pretreatment of an FFPE HeLa cell pellet (including baking, deparaffinization, heat-induced epitope retrieval, and protease treatment), human PPIB target probes were hybridized to the targets by incubating the slides with probe solution for 2 hours at 40 °C, followed by treatment with a pre-pre-amplifiers, pre-amplifiers, and amplifiers for 30 minutes each at 40 °C, and then with two labels, corresponding to Code 8 in Table 6 (Dylight 550 and Dylight 650) for 30 min at 40 °C. Images were obtained on a Leica DM2000 epifluorescence microscope using two different filter sequences; the first being DAPI-GFP-Cy3-Cy5-Cy7, and the second being GFP-DAPI-Cy7-Cy5-Cy3. Individual images are shown in FIG. 15A, overlay images are shown in FIG. 15B, and overlays showing individual “dots” for detection of PPIB RNA are shown in FIG. 15C. These demonstrate that using different detection orders, the disclosed methods can achieve the same level of detection efficiency.

### Example 5

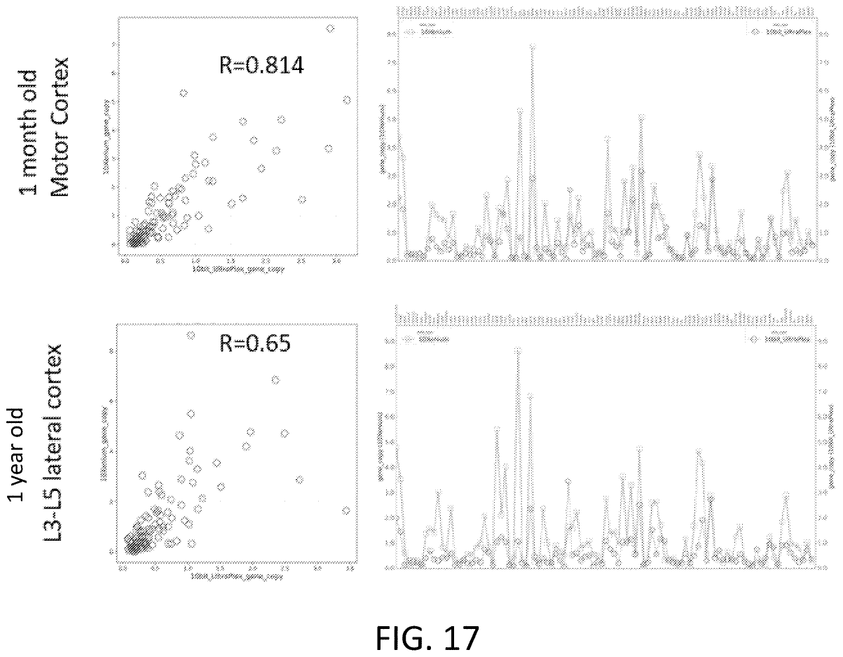

Using 100 target genes screened from a published control assay mouse brain panel, the spatial transcriptomics in a 1 month old C57BL/6 mouse brain and a 1 year old C57BL/6 mouse brain were evaluated (FIG. 17). Each of the 100 target genes is differentially expressed by a unique cell type in the mouse brain and thus can be used to mark the particular cell type. In comparison with the publicly available control data from the same mouse brain tissue, these single-cell gene expression results were significantly correlated with the corresponding control data sets (FIG. 17). The correlation coefficient (R value) ranges from 0.6-0.81, indicating that the Ultraplex assay can effectively profile the gene expressions of mouse brain cells at single-cell level.

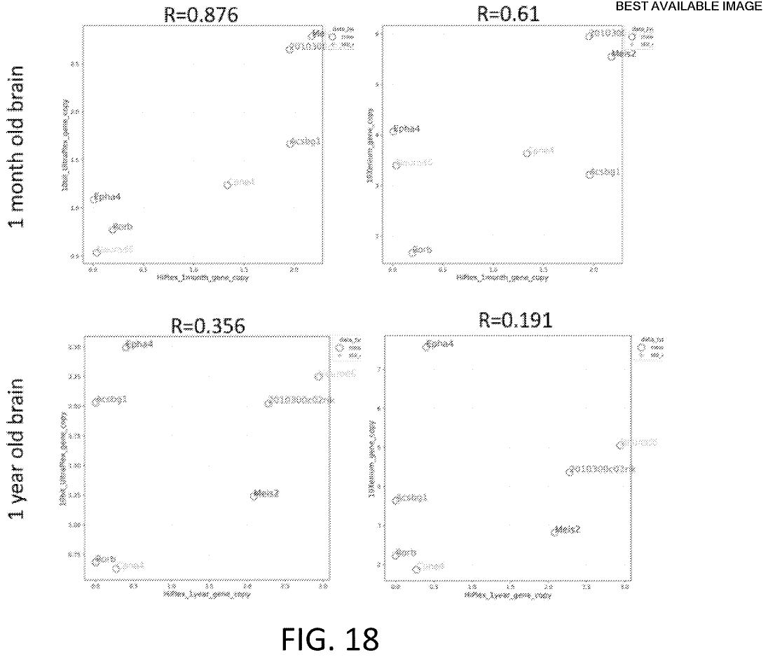

When comparing the minor discrepancies in gene expression between the Ultraplex assay and control assay, it was found that the Ultraplex assay resulted in more accurate measurements of the gene expressions in mouse brain, as suggested by the Hiplex assay performed in the same mouse brain tissues against these disputed genes. In comparison to the gene expression (in copy number/cell) measured by RNAscope Hiplex assay, the control assay tends to overestimate the expressions of certain disputed genes (FIG. 18).

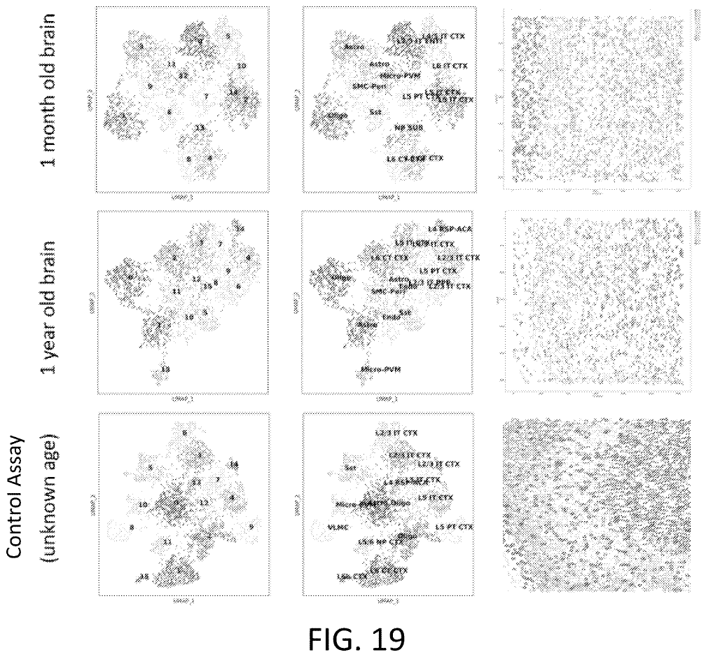

Cell clustering analysis for the Ultraplex “cell×feature” matrix data was also performed, and this analysis successfully identified 10-16 major cell types in different mouse brain regions. Each cell analyzed in the mouse brain slides was assigned with a specific cell type (e.g., L2/3 interneurons, Sst neurons, microglia, etc.), according to the cluster location of the cell in the UMAP. The Ultraplex cell type annotation in the 1 month old and 1 year old mouse brain and their corresponding spatial distribution (laminar locations) were compared with published datasets (Allen brain institute and Xenium public mouse brain datasets), and the results indicated similar cell type annotations and consistent spatial distribution across the mouse brain datasets (FIG. 19).

1. A method for detecting a plurality of target nucleic acids in a sample, the method comprising:
contacting a sample comprising a plurality of target nucleic acids with a plurality of target probe sets, wherein each target probe set is complementary to a target nucleic acid;
contacting the sample with a first plurality of signal generating complexes (SGCs) capable of hybridizing to at least one target probe set, wherein each SGC comprises a plurality of spatially distributed label probes comprising detectable labels;
obtaining a first independent identifier corresponding to at least one target nucleic acid, wherein the first independent identifier is based on the number of distinct detectable labels present in each SGC of the first plurality of SGCs;
removing signals generated by the detectable labels from the sample;
contacting the sample with at least a second plurality of SGCs capable of hybridizing to at least one target probe set, wherein each SGC comprises a plurality of spatially distributed label probes comprising detectable labels; and
obtaining a second independent identifier corresponding to at least one target nucleic acid, wherein the second independent identifier is based on the number of distinct detectable labels present in each SGC of the second plurality of SGCs.

2. The method of claim 1, wherein the method further comprises identifying a target nucleic acid of the plurality of target nucleic acids based on a combination of the first and second independent identifiers.

3. The method of claim 1, wherein the target nucleic acid is identified independent of the order by which the first and second independent identifiers are obtained.

4. The method of claim 1, wherein the first and second independent identifiers are based on the same number of distinct detectable labels present in the first and second plurality of SGCs, respectively.

5. The method of claim 1, wherein the first and second independent identifiers are based on a different number of distinct detectable labels present in the first and second plurality of SGCs, respectively.

6. The method of claim 1, wherein the independent identifier is based on the number of distinct detectable labels present in a combination of at least two SGCs.

7. The method of claim 1, wherein the spatially distributed label probes comprise two, three, four, or five distinct detectable labels.

8.-10. (canceled)

11. The method of claim 1, wherein obtaining an independent identifier comprises obtaining an image of the sample and detecting signals generated by the detectable labels.

12. (canceled)

13. The method of claim 1, wherein each target probe set comprises two or more target probes capable of hybridizing to a target nucleic acid.

14. The method of claim 1, wherein each target probe in the target probe set comprises a T section complementary to a region of a target nucleic acid and an L section complementary to a region of an SGC.

15. The method of claim 14, wherein each T section is complementary to a non-overlapping region of a target nucleic acid and each L section is complementary to a non-overlapping region of an SGC.

16.-17. (canceled)

18. The method of claim 1, wherein each SGC comprises one or more of an amplifier, a pre-amplifier, and a pre-pre-amplifier.

19.-24. (canceled)

25. The method of claim 1, wherein removing the signals generated by the detectable labels comprises removing the SGCs.

26. The method of claim 25, wherein removing the SGCs comprises treatment with an acid reagent that disrupts hybridization between the SGCs and the target nucleic acids, wherein the acid reagent comprises formic acid, acetic acid, propionic acid, butyric acid, valeric acid, oxalic acid, malonic acid, succinic acid, malic acid, tartaric acid, or citric acid.

27.-29. (canceled)

30. The method of claim 1, wherein removing the signals generated by the detectable labels comprises removing the detectable labels from the plurality of label probes using a cleavage reagent.

31.-32. (canceled)

33. The method of claim 1, wherein the sample comprises a cell.

34. The method of claim 33, wherein the method further comprises fixing and/or permeabilizing the cell.

35. The method of claim 1, wherein the target nucleic acid is RNA.

36.-37. (canceled)

38. A method for detecting a plurality of target nucleic acids in a sample, the method comprising:
contacting a sample comprising a plurality of target nucleic acids with a plurality of target probe sets, wherein each target probe set is complementary to a target nucleic acid;
contacting the sample with a plurality of amplification complexes capable of hybridizing to at least one target probe set, wherein each amplification complex comprises a plurality of spatially distributed binding sites for label probes;
contacting the sample with a first plurality of label probes comprising detectable labels, which hybridize to binding sites on the amplification complexes to generate a first plurality of signal generating complexes (SGCs);
obtaining a first independent identifier corresponding to at least one target nucleic acid, wherein the first independent identifier is based on the number of distinct detectable labels present in each SGC of the first plurality of SGCs;
removing signals generated by the detectable labels from the sample;
contacting the sample with at least a second plurality of label probes comprising detectable labels, which hybridize to binding sites on the amplification complexes to generate a second plurality of SGCs; and
obtaining a second independent identifier corresponding to at least one target nucleic acid, wherein the second independent identifier is based on the number of distinct detectable labels present in each SGC of the second plurality of SGCs.

39.-69. (canceled)

70. A kit for carrying out the method of claim 1.
